(19) United States
(12) Patent Application Publication
(10) Pub. No.: US 2016/0000823 A1
(43) Pub. Date: Jan. 7, 2016

(54) METHODS AND COMPOSITIONS FOR IMPROVING HEMOSTASIS

(71) Applicant: Mast Therapeutics, Inc., San Diego, CA (US)

(72) Inventors: R. Martin EMANUELE, San Diego, CA (US); Debra Hoppensteadt, Homer Glen, IL (US); Jawed Fareed, Westchester, IL (US)

(21) Appl. No.: 14/793,730

(22) Filed: Jul. 7, 2015

Related U.S. Application Data
(60) Provisional application No. 62/059,810, filed on Oct. 3, 2014.
(60) Provisional application No. 62/021,676, filed on Jul. 7, 2014.

(51) Int. Cl. A61K 31/77; A61K 38/48; A61K 9/00

(52) U.S. Cl. CPC A61K 31/77; A61K 9/0019; A61K 38/482; C12Y 304/21068

(57) ABSTRACT

Provided are methods and uses of polyoxyethylene/polyoxypropylene copolymers (poloxamers) for treating bleeding and hemorrhage in animals, including human or veterinary subjects, and thus, treating hemostatic dysfunction, resulting from, for example, drug, disease-, trauma- or surgical-induced bleeding. Polyoxyethylene/polyoxypropylene copolymers improve hemostasis and aid in the control of bleeding. Methods for treating strokes using the polyoxyethylene/polyoxypropylene copolymers also are provided. Devices, products and compositions for treating or preventing hemostatic dysfunction are provided.

### RELATED APPLICATIONS

Benefit of priority it claimed to U.S. Provisional Application Ser. No. 62/021,676, to R. Martin Emanuele, Debra Hoppensteadt, Jawed Fareed, filed Jul. 7, 2014, entitled “METHODS AND COMPOSITIONS FOR IMPROVING HEMOSTASIS.” Benefit of priority is claimed to U.S. Provisional Application Ser. No. 62/059,810, to R. Martin Emanuele, Debra Hoppensteadt, Jawed Fareed, filed Oct. 3, 2014, entitled “METHODS AND COMPOSITIONS FOR IMPROVING HEMOSTASIS.”

This application is related to International PCT application No. (Attorney Docket No. 38645.04004.WO01/4004PC) to R. Martin Emanuele, Debra Hoppensteadt, Jawed Fareed, filed the same day herewith, entitled “METHODS AND COMPOSITIONS FOR IMPROVING HEMOSTASIS.”

This application is related to U.S. Provisional Application Ser. No. 62/021,697, to R. Martin Emanuele and Mannarsamy Balasubramanian, filed Jul. 7, 2014, entitled “A POLOXAMER COMPOSITION FREE OF LONG CIRCULATING MATERIAL AND METHODS FOR PRODUCTION AND USES THEREOF.” This application also is related to International PCT Application No. (Attorney Docket No. 38645.4003.WO01/4003PC), to R. Martin Emanuele and Mannarsamy Balasubramanian, filed the same day herewith, and entitled “A POLOXAMER COMPOSITION FREE OF LONG CIRCULATING MATERIAL AND METHODS FOR PRODUCTION AND USES THEREOF.” This application also is related to U.S. application Ser. No. (Attorney Docket No. 38645.4003.US02/4003), to R. Martin Emanuele and Mannarsamy Balasubramanian, filed the same day herewith, and entitled “A POLOXAMER COMPOSITION FREE OF LONG CIRCULATING MATERIAL AND METHODS FOR PRODUCTION AND USES THEREOF.”

The subject matter of each of the above-listed applications is incorporated by reference in its entirety.

### FIELD OF THE INVENTION

Provided herein are methods and compositions and uses thereof for treating hemostatic dysfunction. Provided are methods for treating bleeding and hemorrhage in animals, including human or veterinary subjects, and thus, treating hemostatic dysfunction, resulting from, for example, drug-, disease-, trauma- or surgical-induced bleeding. Polyoxyethylene/polyoxypropylene copolymers improve hemostasis and aid in the control of bleeding. Devices, products and compositions for treating or preventing hemostatic dysfunction are provided.

### BACKGROUND

Hemostasis is the complex physiological process that leads to the cessation of bleeding outside the body or internally in or from blood vessels. Platelets, plasma proteins, and blood vessels and endothelial cells are three components of this process that each play an important role in the events that immediately follow tissue injury and which, under normal circumstances, result in the rapid formation of a clot to halt bleeding. Central to this process is the coagulation cascade, a series of proteolytic events in which certain plasma proteins (or coagulation factors) are sequentially activated in a “cascade” by other previously activated coagulation factors, leading to the rapid generation of thrombin. The large quantities of thrombin produced in this cascade act to cleave fibrinogen into the fibrin peptides that are used in clot formation.

Disturbances to hemostasis can arise due to disease, trauma, surgery, or administration of therapeutic agents that reduce clots or clot formation. For example, it is imperative to control and minimize blood loss during and after surgery. Hemostatic agents such as sutures, fibrin sealants or synthetic glues can be applied to sites of hemorrhage to limit bleeding. Similarly, therapeutic use of anticoagulants, thrombolytics or antithrombotics can cause unwanted bleeding. There is a need for treatments for such hemostatic dysfunction.

### SUMMARY

Provided are methods for treating hemostatic dysfunction and improving hemostasis in a subject in need thereof. Methods for treating stroke and methods for extending the window of treatment with thrombolytic therapy also are provided. Also provided are compositions comprising a polyoxyethylene/polyoxypropylene copolymer for use for treatment and compositions comprising a polyoxyethylene/polyoxypropylene for formulation of a medicament for treatment and prevention of hemostatic dysfunction, treatment of stroke and treatment of stroke in combination with pharmacologic thrombolytic therapy, including extending the time window for pharmacologic thrombolytic therapy. The hemostatic dysfunction can result from surgery or trauma or a clotting disorder or such condition or event. In some embodiments the subject has a disease or a condition such as liver disease, or a genetic disorder such as hemophilia, which is causing or contributing to the hemostatic dysfunction. In some embodiments of treating hemostatic dysfunction the subject has had an acute ischemic stroke (AIS).

The following summary references methods, but it is understood that each method as described also describes a medical use of the a composition comprising a polyoxyethylene/polyoxypropylene copolymer. In all embodiments, including methods and uses, the polyoxyethylene/polyoxypropylene copolymers include those described throughout the disclosure and as follows, and variants thereof. Included in all embodiments are polyoxyethylene/polyoxypropylene copolymer has the formula: HO(C₂H₄O)_{a′}—(C₃H₆O)_{b}—(C₂H₄O)ₐH, where: each a′ and a are the same or different and each is an integer, whereby the hydrophile portion represented by (C₂H₄O) constitutes approximately 60% to 90% or 60%-90% by weight of the compound; and b is an integer, whereby the hydrophobe represented by (C₃H₆O) has a molecular weight of about 1,200 Da to about 2,300 Da or 1,200 to 2,300 Da; the copolymer preparation has been purified to remove low molecular weight impurities; the amount of copolymer administered achieves a circulating Cₘₐₓ concentration of greater than about 1.0 mg/ml; the copolymer preparation has been purified to remove low molecular weight impurities; a′ and a are the same or different and each is an integer, whereby the hydrophile portion represented by (C₂H₄O) constitutes approximately 60% to 90% or 60%-90% by weight of the compound; and b is an integer, whereby the hydrophobe represented by (C₃H₆O) has a molecular weight of about 1,200 Da to about 2,300 Da or 1,200 to 2,300 Da. This includes copolymers where no more than 1.5% or 1% of the total components in the distribution of the copolymer are low molecular weight components having an average molecular weight of less than 4,500 Daltons.

The copolymers include copolymers that have the formula: HO(C₂H₄O)_{a′}—(C₃H₆O)_{b}—(C₂H₄O)ₐH, wherein a′ and a are the same or different and each is an integer from 70 to 105, inclusive; and b is an integer from 15 to 75, inclusive. Also included are the copolymers where the hydrophobe represented by (C₃H₆O) has a molecular weight of about 1,400 Da to 2,000 Da or 1,400 Da to 2,000 Da, and the hydrophile portion constitutes approximately 70% to 90% or 70% to 90% by weight of the copolymer. In all embodiments, included are copolymers in which the low molecular weight impurities are removed or reduced, whereby the polydispersity value is less than or equal to 1.07.

Among the copolymers are those in which the molecular weight of the hydrophobe portion (C₃H₆O) is approximately or is 1,750 Da and the total molecular weight of the copolymer is approximately or is 8,400 to 8,800 Da. Included are poloxamers known to those of skill in the art as poloxamer 188. These include polyoxyethylene/polyoxypropylene copolymers having the formula: HO(C₂H₄O)_{a′}—(C₃H₆O)_{b}—(C₂H₄O)ₐH, where: a′ and a are the same and are 78, 79 or 80; and b is 27, 28, 29 or 30.

Among the polyoxyethylene/polyoxypropylene copolymers for use in the compositions for use and methods, are long circulating material-free (LCMF) poloxamer, particularly LCMF poloxamer 188. Among the LCMF poloxamers is a polyoxyethylene/polyoxypropylene copolymer that has the formula HO(CH₂CH₂O)_{a′}—[CH(CH₃)CH₂O]_{b}—(CH₂CH₂O)ₐH, where each of a and a′ is an integer such that the percentage of the hydrophile (C₂H₄O) is between approximately 60% and 90% by weight of the total molecular weight of the copolymer; a and a′ are the same or different; b is an integer such that the molecular weight of the hydrophobe (C₃H₆O) is between approximately 1,300 and 2,300 Daltons; no more than 1.5% of the total components in the polymeric distribution of the copolymer are low molecular weight components having an average molecular weight of less than 4,500 Daltons; no more than 1.5% of the total components in the polymeric distribution of the copolymer are high molecular weight components having an average molecular weight of greater than 13,000 Daltons; the polydispersity value of the copolymer is less than approximately 1.07 or less than 1.07; and following intravenous administration to a human subject, the circulating plasma half-life of any components not comprising the main peak in the distribution of copolymer is no more than 5.0-fold the circulating half-life of the main component in the distribution of the copolymer. For example, all components comprising the polymeric distribution of the poloxamer copolymer can have a circulating half-life in the plasma of the subject that is no more than 4.0-fold, or 3.0-fold longer than the circulating half-life of the main component of the copolymer following intravenous administration to a human subject. For example, all components in the distribution of the copolymer, when administered to a human subject, have a half-life in the plasma of the subject that is no more than 10 or 12 hours. Included is an LCMF poloxamer where the average molecular weight of the polyoxyethylene/polyoxypropylene copolymer is 8,400-8,800 Daltons.

Among the LCMF poloxamers used in the methods and compositions for use is an LCMF poloxamer is a polyoxyethylene/polyoxypropylene copolymer that has the formula HO(CH₂CH₂O)_{a′}—[CH(CH₃)CH₂O]_{b}—(CH₂CH₂O)ₐH, where each of a and a′ is an integer such that the percentage of the hydrophile (C₂H₄O) is between approximately 60% and 90% by weight of the total molecular weight of the copolymer; a and a′ are the same or different; b is an integer such that the molecular weight of the hydrophobe (C₃H₆O) is between approximately 1,300 and 2,300 Daltons; no more than 1.5% of the total components in the distribution of the copolymer are low molecular weight components having an average molecular weight of less than 4,500 Daltons; no more than 1.5% of the total components in the distribution of the copolymer are high molecular weight components having an average molecular weight of greater than 13,000 Daltons; the polydispersity value of the copolymer is less than approximately 1.07 or less than 1.07; and the LCMF poloxamer 188 is more hydrophilic than a purified poloxamer 188 that contains the long circulating material (LCM).

In another embodiment the LCMF poloxamer is a polyoxyethylene/polyoxypropylene copolymer that has the formula HO(CH₂CH₂O)_{a′}—[CH(CH₃)CH₂O]_{b}—(CH₂CH₂O)ₐH; where each of a and a′ is an integer such that the percentage of the hydrophile (C₂H₄O) is between approximately 60% and 90% by weight of the total molecular weight of the copolymer; a and a′ are the same or different; b is an integer such that the molecular weight of the hydrophobe (C₃H₆O) is between approximately 1,300 and 2,300 Daltons; no more than 1.5% of the total components in the distribution of the copolymer are low molecular weight components having an average molecular weight of less than 4,500 Daltons; no more than 1.5% of the total components in the distribution of the copolymer are high molecular weight components having an average molecular weight of greater than 13,000 Daltons; the polydispersity value of the copolymer is less than approximately 1.07 or less than 1.07; the LCMF poloxamer has a mean retention time (t_{R}) as assessed by reverse phase-high performance liquid chromatography (RP-HPLC) that is shorter than purified LCM-containing poloxamer 188 under the same RP-HPLC conditions; and the capacity factor (k′) of the LCMF poloxamer as assessed by RP-HPLC is less than the k′ for purified LCM-containing poloxamer 188 under the same RP-HPLC conditions.

Methods for producing the poloxamers are described including the LCMF poloxamer. Hence provided are poloxamers produced by a method comprising: a) introducing a poloxamer 188 solution into an extractor vessel, wherein the poloxamer is dissolved in a first alkanol to form a solution; b) admixing the poloxamer solution with an extraction solvent comprising a second alkanol and supercritical carbon dioxide under a temperature and pressure to maintain the supercritical carbon dioxide for a first defined period, where the temperature is above the critical temperature of carbon dioxide but is no more than 40° C.; the pressure is 220 bars to 280 bars; and the alkanol is provided at an alkanol concentration that is 7% to 8% by weight of the total extraction solvent; and c) increasing the concentration of the second alkanol in step b) in the extraction solvent a plurality of times in gradient steps over time of the extraction method, where: each plurality of times occurs for a further defined period; and in each successive step, the alkanol concentration is increased 1-2% compared to the previous concentration of the second alkanol; and d) removing the extraction solvent from the extractor vessel to thereby remove the extracted material from the raffinate poloxamer preparation. In particular embodiments, in step a), the ratio of poloxamer to first alkanol, by weight is about or is from 2:1 to 3:1, inclusive; and/or the plurality of times in step c) occurs in two, three, four or five gradient steps.

Provided are methods for treating, reducing, preventing or reducing the risk of hemostatic dysfunction, which include but is not limited to, conditions involving bleeding or thrombolysis or slow or impaired clotting or other indicia of a risk of bleeding, including in combination with pharmacologic thrombolytic therapy. In some embodiments, the hemostatic dysfunction is manifested as increased bleeding, prolonged blood clotting times or is a parameter associated with a risk of increased bleeding. For example, the hemostatic dysfunction is manifested as increased bleeding or risk thereof, wherein the bleeding is internal or external. The poloxamer is administered at a sufficient dosage to improve hemostasis.

In some embodiments, the polyoxyethylene/polyoxypropylene copolymer is administered at concentrations from about 10.0 mg/mL to about 200.0 mg/mL.

The amount of copolymer administered is sufficient to achieve a circulating Cₘₐₓ concentration, whereby hemostasis is improved. Because lower concentrations of the poloxamer have rheologic actions that can facilitate or synergize actions of thrombolytics and anti-coagulants, the dosage, as shown herein, must be sufficiently high to improve hemostasis by, for example, increasing clotting, or decreasing bleeding or other indicia of hemostatic dysfunction involving increased risk of bleeding or impaired clotting. Such dosages are such that Cₘₐₓ of the poloxamer, is greater than about 1.0 mg/ml or greater than 1.0 mg/ml, particularly greater than 2, 3, 4, 5, 6, 7, 8, 9 and 10 mg/ml, such as at least 7-10 mg/ml. The amount of the copolymer administered can be sufficient to achieve a Cₘₐₓ concentration of greater than 9 mg/ml or 10 mg/ml or a Cₘₐₓ concentration of at least 10 mg/ml. For example, where the copolymer can be an LCMF poloxamer administered to achieve a Cₘₐₓ concentration of greater than 1.5, 2, 3, 4, 5, 6, 7, 8, 9 or 10 mg/ml.

Exemplary of the dosages is at least 400 mg/kg or at least about 400 mg/kg, or 400-1000 mg/kg or at least about 400-1000 mg/kg., or 800-1200 mg/kg or at least about 800-1200 mg/kg, or 800-1000 mg/kg or at least about 800-1000 mg/kg. For treatments and compositions for use herein, a 15% weight of the poloxamer/volume of the composition is administered, such as 15% weight of the copolymer poloxamer/volume of the composition is administered to deliver 400-1000 mg/kg to a subject. The concentration of the polyoxyethylene/polyoxypropylene copolymer in the composition that administered is can be from about 10.0 mg/mL to about 200.0 mg/mL or is 10.0 mg/mL to 200.0 mg/mL. The concentration of the polyoxyethylene/polyoxypropylene copolymer can be from about 0.5% to about 20% by weight/volume, or 1 to 20% weight/volume, or 5-25%, or 10-20%, or 5-15%, or at least 1%, 2%, 3%, 4%, 5%, 6%, 7%, 8%, 9%, 10%, 11%, 12%, 13%, 14%, 15%, 16%, 17%, 18%, 19% or 20%.

The copolymer can be formulated and administered by an suitable route. This includes, parenterally and topically, such as intravenously or subcutaneously or by inhalation. For example, for treatment of hemostatic dysfunction, the copolymer composition is administered intravenously over the course of 1-6 hours or up to 1 hour. The copolymer composition can be administered in volume of about 500 ml to 1000 mL, or in a volume of 500 ml to 1000 mL.

In some embodiments, the subject has recently undergone or is currently undergoing pharmacological thrombolytic therapy. In others the first dose of the poloxamer is administered prior to the pharmacological thrombolytic therapy. It can be administered up to about 5 or 5 hours before the thrombolytic therapy, such as up to 4.5 hours, up to 4 hours, up to 3.5 hours, up to 3 hours, up to 2.5 hours, up to 2 hours, up to 1.5 hours, or up to 1 hour before. In addition, it can be administered following the pharmacological thrombolytic therapy, such at least 2, 3, 4, or 5 hours after the pharmacological thrombolytic therapy.

In some embodiments, the pharmacological thrombolytic therapy is induced by a tissue plasminogen activator (t-PA), anistreplase, streptokinase, urokinase and/or a direct acting thrombolytic. In some instances, the pharmacological thrombolytic therapy is tissue plasminogen activator (t-PA). In some embodiments, the tissue plasminogen activator (t-PA) is alteplase, reteplase and/or tenecteplase. In some embodiments, the direct acting pharmacological thrombolytic therapy is plasmin.

In some embodiments, the subject has recently undergone or is undergoing pharmacological anticoagulant therapy with heparin, low molecular weight heparin, warfarin, Factor Xₐ inhibitors, direct thrombin inhibitors and/or treatment with other such anticoagulant agents. In some embodiments, the subject has recently undergone or is undergoing pharmacological anti-thrombotic therapy with a cyclooxygenase inhibitor, a thromboxane inhibitor, an ADP re-uptake inhibitor or antagonist, a phosphodiesterase inhibitor, a glycoprotein IIb/IIa antagonist or other anti-platelet agent.

In some embodiments, the polyoxyethylene/polyoxypropylene copolymer is administered prophylactically prior to a therapeutic intervention such as surgery or anticoagulant, anti-thrombotic or thrombolytic therapy. In other embodiments, the polyoxyethylene/polyoxypropylene copolymer is administered during or after the therapeutic intervention. In yet other embodiments, the polyoxyethylene/polyoxypropylene copolymer is administered in two doses, where the first dose is administered concomitantly with, the therapeutic intervention. In some instances, the second dose is administered between, for example, 30 minutes and 10 hours, such as 30 minutes to 5 hours, 1 hour to 5 hours, or a suitable time to prevent or reduce the risk of hemostatic dysfunction, after the therapeutic intervention.

In some embodiments, the subject has recently experienced an episode such as a myocardial infarction, a thromboembolic stroke, pulmonary embolism, or other arterial or venous thrombosis.

In some embodiments, the treatment comprises the polyoxyethylene/polyoxypropylene copolymer in an amount of from about 0.5% to about 20% by weight/volume. In some instances, the treatment results in a concentration of the polyoxyethylene/polyoxypropylene copolymer in the circulation of the subject of from about 0.5 mg/mL to about 10.0 mg/mL or from 0.5 mg/mL to 10.0 mg/mL. In other instances, the treatment results in a concentration of the polyoxyethylene/polyoxypropylene copolymer in the circulation of the subject of from about 1 mg/mL to about 5.0 mg/mL. In yet other instances, the treatment results in a concentration of the polyoxyethylene/polyoxypropylene copolymer in the circulation of the subject of greater than 1 mg/mL. In generally the concentrations are at least 5, 7, or 10 mg/mL.

In some embodiments, the subject has undergone or is undergoing or is about to undergo pharmacological thrombolytic therapy, such as treatment with one or more of a tissue plasminogen activator (t-PA), anistreplase, streptokinase, urokinase, or a direct acting thrombolytic, including tissue plasminogen activator (t-PA), such as alteplase, reteplase or tenecteplase. The pharmacological thrombolytic therapy can be direct acting pharmacological thrombolytic therapy, such as administration of plasmin.

Various administration regimens are contemplated. For example, the polyoxyethylene/polyoxypropylene copolymer can be administered before or concomitantly, or after or intermittently with the pharmacological thrombolytic therapy. The polyoxyethylene/polyoxypropylene copolymer can be administered after the pharmacological thrombolytic therapy. For example, in embodiments, where the subject has received or is receiving thrombolytic therapy, the polyoxyethylene/polyoxypropylene copolymer can be administered in two doses, where the first dose is administered concomitantly with the pharmacological thrombolytic therapy or prior to the pharmacological thrombolytic therapy. For example, the first dose of the polyoxyethylene/polyoxypropylene copolymer can be administered up to 5, 4.5, 4, 3.5, 3, 2.5, 2, 1.5 or 1 hour or less before the pharmacological thrombolytic therapy. The second dose can be administered after the pharmacological thrombolytic therapy. For example, the second dose of the polyoxyethylene/polyoxypropylene copolymer can be administered between 30 minutes and 10 hours after the pharmacological thrombolytic therapy.

The subject can be one who has experienced an episode for which thrombolytic therapy is administered, wherein the episode is selected from among a myocardial infarction, a thromboembolic stroke, pulmonary embolism, a deep vein thrombosis, an arterial thrombus and a venous thrombus. The subject can be one who or has been treated with anti-coagulants, such as, for example, anticoagulant therapy with heparin, low molecular weight heparin, warfarin, Factor Xa inhibitors and direct thrombin inhibitors.

Additional hemostatic agents can be administered. For example, the hemostatic agent includes sutures, a fibrin sealant or a synthetic glue.

Also provided are methods for treating a hemorrhagic stroke (HS). The methods include administering to the subject an effective amount of a polyoxyethylene/polyoxypropylene copolymer, where the copolymer is as described above, including LCMF copolymer. This includes copolymers having the following chemical formula: HO(C₂H₄O)_{a′}—(C₃H₆O)_{b}—(C₂H₄O)ₐH, where a′ and a may be the same or different and each is an integer such that the hydrophile portion represented by (C₂H₄O) constitutes approximately 60% to 90% by weight of the compound; and b is an integer such that the hydrophobe represented by (C₃H₆O) has a molecular weight of approximately 1,300 to 2,300 Da, such as approximately 1,500 to 2,100 Da, or about 1,700 to 1,900 Da, wherein the copolymer has been purified to remove certain low molecular weight impurities so that the polydispersity value is less than approximately 1.07.

Also provided are compositions for use and methods for treating an acute ischemic stroke (AIS). The method includes administering to the subject an effective amount of a polyoxyethylene/polyoxypropylene copolymers as described above and herein, where the

subject has excess bleeding as a consequence of recently undergoing or is currently undergoing pharmacological thrombolytic therapy, to treat the AIS. For all embodiments, including all methods and uses and combinations and compositions, the polyoxyethylene/polyoxypropylene copolymer can be a long-circulating material-free (LCMF) poloxamer as described herein. For example, the LCMF poloxamer includes those described above. Included are
embodiments, where the LCMF poloxamer for use in the methods and compositions and uses, is such that all components in the distribution of the copolymer, when administered to a subject, have a circulating half-life in the plasma of the subject that is no more than 4.0-fold, 3.0-fold, 2.0 fold or 1.5-fold longer than the circulating half-life of the main component in the distribution of the copolymer. In particular embodiments, all components in the distribution of the copolymer, when administered to a subject, have a circulating half-life in the plasma of the subject that is no more than 1.5-fold longer than the circulating half-life of the main component in the distribution of the copolymer. The LCMF poloxamer is one in which all of the components of the polymeric distribution clear from the circulation at approximately the same rate, such as where all components in the distribution of the copolymer, when administered to a subject, have a circulating half-life in the plasma of the subject that is no more than the circulating half-life of the main component in the distribution of the copolymer. For example, all components in the distribution of the copolymer, when administered to a human subject, have a circulating half-life in the plasma of the subject that is no more than 30 hours, 25 hours, 20 hours, 15 hours, 12 hours, 10 hours, 9 hours, 8 hours or 7 hours, or, for example, all components in the distribution of the copolymer, when administered to a human subject, have a half-life in the plasma of the subject that is no more than 10 hours or 12 hours.

The LCMF polyoxyethylene/polyoxypropylene copolymer has the above noted formula, where hydrophobe has a molecular weight of about 1,400 to 2,000 Da or 1,400 to 2,000 Da, and a hydrophile portion constituting approximately 70% to 90% or 70% to 90% by weight of the copolymer. In some embodiments, the molecular weight of the hydrophobe (C₃H₆O) is about or is 1,750 Da; in others or the same embodiments, the average molecular weight of the polyoxyethylene/polyoxypropylene copolymer is 7680 to 9510 Daltons, such as about or at 8,400-8,800 Daltons. In the LCMF poloxamer, the percentage of high molecular weight components with a molecular weight of greater than or equal to 13,000 Daltons constitute less 1% of the total distribution of components of the poloxamer preparation; such preparation does not, when administered to a subject, result in a component with the longer half-life. In some embodiments, the percentage of high molecular weight components in the preparation greater than 13,000 Daltons constitutes less than 0.9%, 0.8%, 0.7%, 0.6%, 0.5% or less of the total distribution of components of the poloxamer preparation, and, that when administered, does not result in a component with the longer half-life. For all embodiments of the method herein, including the LCMF poloxamer, such as the LCMF P-188, as well as the compositions and uses, the poloxamer can have a polydispersity value of the polyoxyethylene/polyoxypropylene copolymer that is less than 1.06, 1.05, 1.04, 1.03 or less. The LCMF poloxamer can be an LCMF P-188 poloxamer. It can be produced by any suitable method that removes long circulating material. The methods include those described herein. In some embodiments of AIS, where the a polyoxyethylene/polyoxypropylene copolymer is administered, the pharmacological thrombolytic therapy is administered immediately after the AIS and up to about 10 hours after the AIS. In other embodiments, the pharmacological thrombolytic therapy is administered 3.5 hours after the AIS and up to about 10 hours after the AIS, such as 5, 4.5, or 4 hours after the stroke. Further treatment with the copolymer an be administered at least 6, 7, 8, 9 or 10 hours after the stroke and after the pharmacological thrombolytic therapy.

In some embodiments, the pharmacological thrombolytic therapy, without excluding intracranial hemorrhage, is administered to the subject.

In embodiments in which the patient has a high risk of bleeding the polyoxyethylene/polyoxypropylene copolymer is administered. The high risk of bleeding can be located at, for example, a site of recent surgery, an intracranial site, a gastrointestinal site, a urogenital site and a respiratory tract site.

In some embodiments, the pharmacological thrombolytic therapy is tissue plasminogen activator (t-PA), anistreplase, streptokinase, urokinase and a direct acting thrombolytic. In some instances, the pharmacological thrombolytic therapy is tissue plasminogen activator (t-PA). In some embodiments, the tissue plasminogen activator (t-PA) is selected from among alteplase, reteplase and tenecteplase. In some embodiments, the direct acting pharmacological thrombolytic therapy is plasmin.

In some embodiments, the polyoxyethylene/polyoxypropylene copolymer that is administered has a concentration from about or at 10.0 mg/mL to about or at 200.0 mg/mL. In some instances, the treatment comprises the administering the polyoxyethylene/polyoxypropylene copolymer in an amount of from about 0.5% to about 20% by weight/volume. In some instances, the treatment results in a concentration of the polyoxyethylene/polyoxypropylene copolymer in the circulation of the subject of from about or at 0.5 mg/mL to about or at 10.0 mg/mL. In other instances, the treatment is administered to result in a concentration of the polyoxyethylene/polyoxypropylene copolymer in the circulation of the subject of from about 1.0 mg/mL to about 5.0 mg/mL. In yet other instances, the composition is administered in an amount to result in a concentration of the polyoxyethylene/polyoxypropylene copolymer in the circulation of the subject of more than 1 mg/mL up to 10 mg/mL.

Provided are methods of treating stroke by administering a composition to a subject, comprising a polyoxyethylene/polyoxypropylene copolymer after the stroke, but prior to further treatment. The stroke can be an acute ischemic stroke (AIS) or a hemorrhagic stroke. The stroke and be an AIS, and the further treatment can pharmacological thrombolytic therapy as described above and herein.

Provided are methods and uses of the polyoxyethylene/polyoxypropylene copolymers for

extending the pharmacologic therapy treatment window for ischemic stroke by administering the polyoxyethylene/polyoxypropylene copolymer to a subject after the stroke; and
then administering the pharmacological thrombolytic therapy. Administration of the polyoxyethylene/polyoxypropylene copolymer can be repeated after administration of the pharmacological thrombolytic therapy. The thrombolytic therapy can be any known to those of skill in the art, including any described herein,

Also provided are kits and combinations that contain a composition containing a the polyoxyethylene/polyoxypropylene copolymer as described herein; and second compositions containing an additional hemostatic agent. Additional hemostatic agents, include, for example, a fibrin sealant, sutures, synthetic glue or a bandage. The copolymer can be adsorbed to the hemostatic agent or mixed therewith. Provided is a fibrin glue or sealant, comprising 10-20 mg/ml of the copolymer.

Additional embodiments are contemplated and described below and set forth in the claims.

### BRIEF DESCRIPTION OF THE DRAWINGS

FIG. 1 is a general process 100 for supercritical fluid extraction (SFE) of a poloxamer.

FIG. 2 is a specific exemplary process 100′ for preparing a poloxamer, such as poloxamer 188, using the methods described herein.

FIG. 3 is a specific exemplary process 100″ for preparing apoloxamer, such as poloxamer 188, using methods described herein.

FIG. 4 shows an extraction apparatus useful in the methods provided herein.

FIG. 5 shows one embodiment of the cross section of stainless spheres of different sizes in a solvent distribution bed.

FIG. 6A-B shows a gel permeation chromatography (GPC) comparison of low molecular weight substance content in a commercially available poloxamer 188 (Panel A) versus a material purified according to an embodiment provided herein (Panel B).

FIG. 7A-B shows enlarged HPLC-GPC chromatograms depicting the molecular weight distribution of components in plasma over time.

FIG. 8A-B shows individual plasma concentrations of Poloxamer 188 (Panel A) and high molecular weight component (Panel B) in healthy humans during and following a 48 hour continuous IV infusion of purified poloxamer 188 as described in Grindel et al. (2002) (Biopharmaceutics & Drug Disposition, 23:87-103).

FIG. 9 shows a Reverse Phase High Performance Liquid Chromatography (RP-HPLC) chromatogram comparing profiles of compositions of 15% LCMF 188 with 15% P188 (available under the trademark Flocor®), relative to other poloxamers and polymers (of different hydrophobicity/hydrophilicity) showing that the LCMF 188 is more hydrophilic than the P188.

FIG. 10 shows a RP-HPLC chromatogram comparing different lots of LCMF poloxamer 188 with purified poloxamer 188 confirming the difference in hydrophilicity.

FIG. 11 depicts the coagulation cascade, with the intrinsic pathway and extrinsic pathway converging at Factor X to initiate the common pathway, containing additional coagulation factors and cofactors, resulting in fibrinogen cleavage to form fibrin (soluble), which polymerizes and forms a fibrin clot (insoluble fibrin) during hemostasis. Also depicted is the thrombolysis pathway, whereby enzymes cleave plasminogen precursor to form the active enzyme, plasmin, which leads to cleavage of the fibrin network (fibrinolysis) and dissolution of the clot (thrombolysis).

FIG. 12 illustrates the fibrokinetics of heparinaized plasma supplemented with Dextran 40 (diamond), and Dextran 70 (square), poloxamer 188 (triangles), or saline (control; X).

FIG. 13 illustrates the polymerization of fibrin in plasma from patients with liver disease (icteric plasma samples), treated with 0.3 mg/mL, 0.6 mg/mL, 1.25 mg/mL, 2.5 mg/mL, 5 mg/mL or 10 mg/mL poloxamer 188.

FIG. 14 depicts the times required for cessation of amputated tail bleeding of rats treated with saline alone, tissue plasminogen activator (t-PA) alone (1 mg/kg), tranexamic acid (TA) alone (5 mg/kg), or t-PA (1 mg/kg) in combination with TA (5 mg/kg or 10 mg/kg).

FIG. 15 depicts the times required for cessation of amputated tail bleeding of rats treated with saline alone, tissue plasminogen activator (t-PA) alone (1 mg/kg), or t-PA (1 mg/kg) in combination with poloxamer 188 (P188) (10 mg/kg, 2.5 mg/kg or 1.25 mg/kg).

### DETAILED DESCRIPTION

### Outline

A. DEFINITIONS

B. HEMOSTASIS AND POLOXAMERS
1. Hemostasis and hemostatic dysfunction
   a. Primary Hemostasis
      i. Platelet adhesion
      ii. Platelet activation
      iii. Platelet aggregation
   b. Secondary Hemostasis and the Coagulation Cascade
2. Poloxamers and hemostasis

C. POLOXAMERS AND PURIFIED POLOXAMERS
1. Poloxamer 188 (P188)
2. Molecular Diversity of Poloxamer 188
   a. Low Molecular Weight Components
   b. Components Resulting in Long Circulating Half-Life
3. Long Circulating Material Free (LCMF) Poloxamer
4. Extraction Method For Purifying Poloxamers
   a. Processes For Extraction
      i. Supercritical Methods
      ii. High Pressure Methods
   b. Extraction Vessel and System
   c. Extraction and Removal of Extractants
   d. Exemplary Methods
      i. Removal of Low Molecular Weight (LMW) Components
      ii. Preparation of Long Circulating Material Free (LCMF) Poloxamer
      iii. Methods for Confirming the Identity of LCMF Poloxamers

D. PHARMACEUTICAL COMPOSITIONS AND FORMULATIONS
1. Formulations
2. Dosage
3. Administration
   a. Systemic Administration
   b. Local Administration
4. Articles of Manufacture and Kits
   a. Sutures
   b. Tissue Adhesives
   c. Dressings
   d. Gelatin Sponges
   e. Kits

E. METHODS OF ASSESSING HEMOSTASIS

F. METHODS OF PROMOTING HEMOSTASIS
1. Subject Selection
   a. Subjects Receiving Thrombolytic, Anticoagulant, or Antithrombotic Therapy
   b. Subjects Receiving Hemostatic Agents
2. Combination Therapies

G. METHODS OF TREATING STROKE

H. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, “hemostasis” refers to balance between bleeding and thrombosis in order to maintain and blood flow in an organ or body part. Hemostasis encompasses the process of blood clotting to prevent blood loss following blood vessel injury to subsequent dissolution of the blood clot following tissue repair. The process and components, which are known to those of skill in the art, are described below in Section C. Hemostasis, when not disrupted, is regulated in the body, because insufficient blood clotting can lead to bleeding, including bleeding disorders such as hemophilia. Over-active blood clotting also can be problematic, causing or participating in ischemic disorders. For example, abnormal, over-active blood clotting can lead to thrombosis, which results in obstructed blood flow through the circulatory system and can cause embolisms.

As used herein, “hemostatic dysfunction” refers to disruption of hemostasis. For purposes herein, it refers to changes in the process, or any change or problem or impairment with any step in hemostasis that is manifested as increased bleeding, prolonged blood clotting time, or similar indicia, such as the result of an laboratory assay indicative of increased bleeding risk or reduced clotting, such as that which occurs from bleeding disorders or administration of anti-coagulant agents or anti-thrombotic agents. The bleeding refers to internal or external bleeding. By administering a poloxamer as described herein and at the dosages herein, the dysfunction can be reduced or eliminated.

As used herein, “clotting” or “coagulation” refers to the formation of an insoluble fibrin clot, or the process by which the hemostasis is initiated, ultimately resulting in the formation of an insoluble fibrin clot. “Coagulation is a process by which blood forms clots. It is an important part of hemostasis, including the cessation of blood loss from a damaged vessel, where a damaged blood vessel wall is covered by a platelet and fibrin-containing clot to stop bleeding and begin repair of the damaged vessel. Disorders of coagulation can lead to an increased risk of bleeding (hemorrhage) or obstructive clotting (thrombosis). The coagulation pathway is highly conserved, and involves cellular (platelet) and protein (coagulation factor) components.

As used herein, “procoagulant” refers to any substance that promotes blood coagulation.

As used herein, “thrombolytic therapy” is a treatment involving administration of a thrombolytic agent to dissolve blood clots (e.g., thrombi or emboli) in blood vessels by thrombolysis.

As used herein, “anticoagulant” refers to any substance that inhibits blood coagulation.

As used herein, “bleeding disorder” refers to a condition in which the subject has a decreased ability to control bleeding. Bleeding disorders can be inherited or acquired, and can result from, for example, defects or deficiencies in the coagulation pathway, defects or deficiencies in platelet activity, or vascular defects.

As used herein, “an ischemic stroke” is a stroke in which blood flow to or in the brain is cut off by clot. An ischemic stroke can occur as embolic and as thrombotic strokes. In an embolic stroke, a blood clot (embolus) forms somewhere in the body, such as the heart, and travels through the bloodstream to the brain where it encounters a blood vessel small enough to block its passage so that it lodges there, blocking the blood vessel and causing a stroke. The medical word for this type of blood clot is embolus. In a thrombolic stroke, blood flow is impaired because of a blockage to one or more of the arteries supplying blood to the brain. The blockage caused by thrombosis that produces a clot on a blood-vessel deposit is thrombus.

As used herein, poloxamers are synthetic block copolymers of ethylene oxide and propylene oxide. A “polyoxyethylene/poloxypropylene copolymer,” “PPC” or “poloxamer” refers to a block copolymer containing a central block of polyoxypropylene (POP) flanked on both sides by blocks of polyoxyethylene (POE) having the following chemical formula:

HO(C₂H₄O)_{a′}—[C₃H₆O]_{b}—(C₂H₄O)ₐH

where: a′ and a can be the same or different and each is an integer such that the hydrophile portion represented by (C₂H₄O) (i.e. the polyoxyethylene portion of the copolymer) constitutes approximately 60% to 90% by weight of the copolymer, such as 70% to 90% by weight of the copolymer; and b is an integer such that the hydrophobe represented by (C₃H₆O)_{b} (i.e., the polyoxypropylene portion of the copolymer) has a molecular weight of approximately 950 to 4,000 Daltons (Da), such as about 1,200 to 3,500 Da, for example, 1,200 to 2,300 Da, 1,500 to 2,100 Da, 1,400 to 2,000 Da or 1,700 to 1,900 Da. For example, the molecular weight of the hydrophile portion can be between 5,000 and 15,000 Da. Exemplary poloxamers having the general formula described above include poloxamers wherein a or a′ is an integer 5-150 and b is an integer 15-75, such as poloxamers wherein a is an integer 70-105 and b is an integer 15-75. Poloxamers include poloxamer 188 (e.g., those sold under the trademarks Pluronic® F-68, Flocor®, Kolliphor® and Lutrol®).

The nomenclature of the polyoxyethylene/polyoxypropylene copolymer relates to its monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 188 describes a polymer containing a polyoxypropylene hydrophobe of about 1,800 Da with a hydrophilic polyoxyethylene block content of about 80% of the total molecular weight.

Poloxamers can be synthesized in two steps, first by building the polyoxypropylene core, and then by addition of polyoxyethylene to the terminal ends of the polyoxypropylene core. Because of variation in the rates of polymerization during both steps, a poloxamer can contain heterogeneous polymer species of varying molecular weights. The distribution of polymer species can be characterized using standard techniques including, but not limited to, gel permeation chromatography (GPC).

As used herein, Poloxamer 188 (also called P188 or P188) refers to a polyoxyethylene/polyoxypropylene copolymer that has the following chemical formula:

HO(CH₂CH₂O)_{a′}—[CH(CH₃)CH₂O]_{b}—(CH₂CH₂O)ₐH, where:

a′ and a can be the same or different and each is an integer such that the hydrophile portion represented by (C₂H₄O) (i.e. the polyoxyethylene portion of the copolymer) constitutes approximately 60% to 90%, such as approximately 80% or 81%; and b is an integer such that the hydrophobe represented by (C₃H₆O) has a molecular weight of approximately 1,300 to 2,300 Da, such as 1,400 to 2,000 Da, for example approximately 1,750 Da. For example, a is about 79 and b is approximately or is 28. The average total molecular weight of the compound is approximately 7,680 to 9,510 Da, such as generally 8,400-8,800 Da, for example about or at 8,400 Da. Poloxamer 188 is a preparation that can contain a heterogeneous distribution of polymer species that primarily vary in overall chain length of the polymer, but also include truncated polymer chains with unsaturation, and certain low molecular weight glycols. Included among poloxamer 188 molecules are those that exhibit a species profile (e.g., determined by GPC) containing a main peak and “shoulder” peaks on both sides representing low molecular weight (LMW) polymer species and high molecular weight (HMW) polymer species. Poloxamer 188 also refers to materials that are purified to remove or reduce species other than the main component.

As used herein, “main component” or “main peak” with reference to a poloxamer 188 preparation refers to the species of copolymer molecules that have a molecular weight of less than about 13,000 Da and greater than about 4,500 Da, with an average molecular weight of between about 7,680 to 9,510 Da, such as generally 8,400-8,800 Da, for example about or at 8,400 Da. Main peak species include those that elute by gel permeation chromatography (GPC) at between 14 and 15 minutes depending on the chromatography conditions (see U.S. Pat. No. 5,696,298).

As used herein, “low molecular weight” or “LMW” with reference to species or components of a poloxamer 188 preparation refers to components that have a molecular weight generally less than 4,500 Da. LMW species include those that elute by gel permeation chromatography (GPC) after 15 minutes depending on the chromatography conditions. (see U.S. Pat. No. 5,696,298). Such impurities can include low molecular weight poloxamers, poloxamer degradation products (including alcohols, aldehydes, ketones, and hydroperoxides), diblock copolymers, unsaturated polymers, and oligomeric glycols including oligo(ethylene glycol) and oligo(propylene glycol).

As used herein, “high molecular weight” or “HMW” with reference to species or components of a poloxamer 188 preparation refers to components that have a molecular weight generally greater than 13,000 Da, such as greater than 14,000 Da, greater than 15,000 Da, greater than 16,000 Da or greater. HMW species include those that elute by gel permeation chromatography (GPC) at between 13 and 14 minutes depending on the chromatography conditions (see U.S. Pat. No. 5,696,298).

As used herein, “polydispersity” or “D” refers to the breadth of the molecular weight distribution of a polymer composition. A monodisperse sample is defined as one in which all molecules are identical. In such a case, the polydispersity (Mw/Mn) is 1. Narrow molecular weight standards have a value of D near 1 and a typical polymer has a range of 2 to 5. Some polymers have a polydispersity in excess of 20. Hence, a high polydispersity value indicates a wide variation in size for the population of molecules in a given preparation, while a lower polydispersity value indicates less variation. Methods for assessing polydispersity are known in the art, and include methods as described in U.S. Pat. No. 5,696,298. For example, polydispersity can be determined from chromatograms. It is understood that polydispersity values can vary depending on the particular chromatogram conditions, the molecular weight standards and the size exclusion characteristics of gel permeation columns employed. For purposes herein, reference to polydispersity is as employed in U.S. Pat. No. 5,696,298, as determined from chromatograms obtained using a Model 600E Powerline chromatographic system equipped with a column heater module, a Model 410 refractive index detector, Maxima 820 software package (all from Waters, Div. of Millipore, Milford, Mass.), two LiChrogel PS-40 columns and a LiChrogel PS-20 column in series (EM Science, Gibbstown, N.J.), and polyethylene glycol molecular weight standards (Polymer Laboratories, Inc., Amherst, Mass.). It is within the level of a skilled artisan to convert any polydispersity value that is obtained using a different separation method to the values described herein simply by running a single sample on both systems and then comparing the polydispersity values from each chromatogram.

As used herein, “purified poloxamer 188” or “P188-P” or “purified long circulating material (LCM)-containing poloxamer 188” refers to a poloxamer 188 that has polydispersity value of the poloxamer of less than or about 1.07, such as less than or 1.05 or less than or 1.03, and is a purified poloxamer 188 that has a reduced amount of low molecular weight components, but contains the long circulating material. A poloxamer 188 in which “low molecular weight material has been removed” or “low molecular weight material has been reduced,” or similar variations thereof, refers to a purified poloxamer 188 in which there is a distribution of low molecular weight components of no more than or less than 3.0%, and generally no more than or less than 2.0% or no more than or less than 1.5% of the total distribution of components. Typically, such a poloxamer 188 exhibits reduced toxicity compared to forms of poloxamer 188 that contain a higher or greater percentage of low molecular weight components. The poloxamer 188 is purified to remove or reduce low molecular weight components. Commercially available and prior preparations of poloxamer, such as poloxamer 188, have a long circulating material (LCM) that, when administered to a human, has a half life that is more than 5.0 fold the circulating half-life of the main component in the distribution of the copolymer.

An exemplary purified LCM-containing poloxamer 188 is poloxamer 188 available under the trademark FLOCOR® (see, also U.S. Pat. No. 5,696,298, which describes LCM-containing poloxamer 188). When the purified LCM-containing poloxamer 188 is administered as an intravenous injection to a mammal, particularly a human, GPC analysis of blood obtained from the treated subject exhibits two circulating peaks: a peak designated the main peak that comprises the main component of the polymeric distribution and a peak of higher molecular weight, compared to the main peak, that exhibits a substantially slower rate of clearance (more than 5-fold slower than the main peak, typically more than 30 hours and as much as 70 hours, as shown herein) from the circulation, i.e., a long circulating material (LCM).

As used herein, long circulating material (LCM) refers to material in prior poloxamer preparations that, upon administration to a subject, have a half-life in the subject, such as a human, that is substantially longer than the half-life of the main component of the poloxamer preparation. When administered to a human subject the LCM material in a poloxamer preparation has more than about or more than 5-fold the half life of the main component of the poloxamer preparation. The LCMF poloxamers as provided herein do not give rise to such long circulating material. There is no component that has a half-life that it 5-fold longer than the main component. For comparing poloxamers, components of corresponding poloxamers are compared, where a corresponding poloxamers have the same formula. For example, an LCMF poloxamer 188 is compared to a poloxamer 188.

As used herein, “long circulating material free” or “LCMF” with reference to a poloxamer, such as poloxamer 188, refers to a purified poloxamer 188 preparation that has a reduced amount of low molecular weight components, as described above for purified poloxamer 188, and that, following intravenous administration to a subject, the components of the polymeric distribution clear from the circulation in a more homogeneous manner such that any long circulating material exhibits a half-life (in human subjects) that is no more than 5-fold longer than the circulating half-life (t_{1/2}) of the main peak. Thus, an LCMF poloxamer, including an LCMF poloxamer 188, is a poloxamer that does not contain components, such as a high molecular weight components or low molecular weight components as described herein, that are or gives rise to a circulating material with a t_{1/2} that, when administered to a human subject, is more than 5.0-fold greater than the t_{1/2} of the main component, and generally no more than 4.0, 3.0, 2.0 or 1.5 fold greater than the half-life of the main component in the distribution of the copolymer. Typically, an LCMF poloxamer is a poloxamer in which all of the components of the polymeric distribution clear from the circulation at a more homogeneous rate than prior preparations of poloxamer.

As used herein, “distribution of copolymer” refers to the molecular weight distributions of the polymeric molecules in a poloxamer preparation. The distribution of molecular masses can be determined by various techniques known to a skilled artisan, including but not limited to, colligative property measurements, light scattering techniques, viscometry and size exclusion chromatography. In particular, gel permeation chromatography (GPC) methods can be employed that determine molecular weight distribution based on the polymer's hydrodynamic volume. The distribution of molecular weight or mass of a polymer can be summarized by polydispersity. For example, the greater the disparity of molecular weight distributions in a poloxamer, the higher the polydispersity.

As used herein, half-life, biological half-life, plasma half-life, terminal half-life, elimination half-life or t_{1/2} refer to the time that a living body requires to eliminate one half of the quantity of an administered substance through its normal channels of elimination. The normal channels of elimination generally include the body's cleansing through the function of kidneys and liver in addition to excretion functions to eliminate a substance from the body. Half-life can be described as the time it takes the blood plasma concentration of a substance to halve its steady state level, i.e. the plasma half-life. A half-life can be determined by giving a single dose of drug, usually intravenously, and then the concentration of the drug in the plasma is measured at regular intervals. The concentration of the drug will reach a peak value in the plasma and will fall as the drug is broken down and cleared from the blood.

As used herein “Cmax” refers to the peak or maximal plasma concentration of a drug after administration.

As used herein, the “concentration of a drug at steady state” or “Css” refers to the concentration of drug at which the rate of drug elimination and drug administration are equal. It is achieved generally following the last of an infinite number of equal doses given at equal intervals. The time required to achieve a steady state concentration depends on the half-life of the drug. The shorter the half-life, the more rapidly steady state is reached. Typically it takes 3-5 half-lives to accumulate to greater than 90% of the final steady state concentrations.

As used herein, “impurities” refer to unwanted components in a poloxamer preparation. Typically impurities include LMW components less than 4,500 Daltons and high molecular weight components greater than 13,000 Daltons.

As used herein, “remove or reduce” with reference to a poloxamer component in a preparation refers to decreasing the weight percentage of the component in the poloxamer preparation relative to the initial percentage of the component. Generally, a poloxamer component is removed or reduced if the percentage by weight of the component to the total distribution of components is decreased by at least 1%, and typically at least 2%, 3%, 4%, 5%, or more. For example, most commercial preparations of a poloxamer 188 contain a LMW component (less than 4,500 Daltons) that is about 4% by weight of the total components in the distribution. The LMW component is reduced in a purified product if there is less than 3% by weight of the component, such as less than 2% or 1%.

As used herein, “solvent” refers to any liquid in which a solute is dissolved to form a solution.

As used herein, a “polar solvent” refers to a solvent in whose molecules there is either a permanent separation of positive and negative charges, or the centers of positive and negative charges do not coincide. These solvents have high dielectric constants, are chemically active, and form coordinate covalent bonds. Examples of polar solvents are alcohols and ketones.

As used herein, “feed” refers to a solute dissolved in a solvent.

As used herein, an “extraction solvent” refers to any liquid or supercritical fluid that can be used to solubilize undesirable materials that are contained in a poloxamer preparation. It is a solvent that can effect solvent extraction to separate a substance from one or more others based on variations in the solubilities. Generally an extraction solvent is immiscible or partially miscible with the solvent in which the substance of interest is dissolved. For example, an extraction solvent is one that does not mix or only partially mixes with a first solvent in which the substance of interest is dissolved, so that, when undisturbed, two separate layers form. Exemplary extraction solvents are supercritical liquids or high pressure liquids.

As used herein, the terms “supercritical liquid” and “supercritical fluid” include any compound, such as a gas, in a state above its critical temperature (T_{c}; i.e. the temperature, characteristic of the compound, above which it is not possible to liquefy the compound) and critical pressure (p_{c}; i.e., the minimum pressure which would suffice to liquefy the compound at its critical temperature). In this state, distinct liquid and gas phases typically do not exist. A supercritical liquid typically exhibits changes in solvent density with small changes in pressure, temperature, or the presence of a co-modifier solvent.

As used herein, “supercritical carbon dioxide” refers to a fluid state of carbon dioxide where it is held at or is above its critical temperature (about 31° C.) and critical pressure (about 74 bars). Below its critical temperature and critical pressure, carbon dioxide usually behaves as a gas in air or as a solid, dry ice, when frozen. At a temperature that is above 31° C. and a pressure above 74 bars, carbon dioxide adopts properties midway between a gas and a liquid, so that it expands to fill its container like a gas but with a density like that of a liquid.

As used herein, “critical temperature” or “critical point” refers to the temperature that denotes the vapor-liquid critical point, above which distinct liquid and gas phases do not exist. Thus, it is the temperature at and above which vapor of the substance cannot be liquified no matter how much pressure is applied. For example, the critical temperature of carbon dioxide is about 31° C.

As used herein, “critical pressure” refers to the pressure required to liquefy a gas at its critical temperature. For example, the critical pressure of carbon dioxide is about 74 bars.

As used herein, the term “high pressure liquid” includes a liquid formed by pressurizing a compressible gas into the liquid at room temperature or a higher temperature.

As used herein, a “co-modifier solvent” refers to a polar organic solvent that increases the solvent strength of an extraction solvent (e.g., supercritical fluid carbon dioxide). It can interact strongly with the solute and thereby substantially increase the solubility of the solute in the extraction solvent. Examples of co-modifier solvents include alkanols. Typically between 5% and 15% by weight of co-modified solvent can be used.

As used herein, the term “alkanol” includes simple aliphatic organic alcohols. In general, the alcohols intended for use in the methods provided herein include six or fewer carbon atoms (i.e., C₁-C₆ alkanols). The alkane portion of the alkanol can be branched or unbranched. Examples of alkanols include, but are not limited to, methanol, ethanol, isopropyl alcohol (2-propanol), and tert-butyl alcohol.

As used herein, “subcritical extraction” refers to processes using a fluid substances that would usually be gaseous at normal temperatures and pressures, that are converted to liquids at higher pressures and lower temperatures. The pressures or temperatures are then normalized and the extracting material is vaporized leaving the extract. Extractant can be recycled.

As used herein, “extraction vessel” or “extractor” refers to a high-pressure vessel that is capable of withstanding pressures of up to 10,000 psig and temperatures of up to 200° C. The volume of the vessels can range from 2 mL to 200 L, and generally is 1 L to 200 L, such as 5 L to 150 L. Extraction vessels generally are made out of stainless steel. Such devices are well known to a skilled artisan and available commercially.

As used herein, “isocratic” refers to a system in which an extraction solvent is used at a constant or near constant concentration.

As used herein, “gradient” or “gradient steps” refers to a system in which two or more extraction solvents are used that differ in their composition of components, typically by changes in concentration of one or more components. For example, the concentration of the alkanol solvent (e.g., methanol) is successively increased during the course of the extraction. Thus, the extraction solvent does not remain constant.

As used herein, “plurality” refers to a number of iterations of a process or step. The number of repeats can be 2, 3, 4, 5, 6 or more.

As used herein, “extracted material” refers to the product containing the removed materials.

As used herein, “raffinate” refers to a product which has had a component or components removed. For example, the purified poloxamer in which extracted material has been removed.

As used herein, “batch method” or “batch extraction” refers to a process of extracting the solute from one immiscible layer by shaking the two layers until equilibrium is attained, after which the layers are allowed to settle before sampling. For example, a batch extraction can be performed by mixing the solute with a batch of extracting solvent. The solute distributes between the two phases. Once equilibrium is achieved, the mixing is stopped and the extract and raffinate phases are allowed to separate. In this method, the spent solvent can be stripped and recycled by distillation or fresh solvent can be added continuously from a reservoir.

As used herein, a “continuous method” or “continuous extraction” refers to a process in which there is a continuous flow of immiscible solvent through the solution or a continuous countercurrent flow of both phases. For example, a continuous extracting solvent is mixed with the solute. The emulsion produced in the mixer is fed into a settler unit where phase separation takes place and continuous raffinate and extract streams are obtained.

As used herein, “pharmaceutical composition” includes a composition comprising a polyoxyethylene/polyoxypropylene copolymer described herein, such as an LCMF poloxamer, formulated as a pharmaceutically acceptable formulation and/or with one or more pharmaceutically acceptable excipients. In certain instances, the pharmaceutical composition comprises an aqueous injectable solution of the poloxamer buffered at a desired pH, such as 6-7 or 6 or about 6, with a suitable buffer. Exemplary of buffers are any known to those of skill in the art to be biocompatible, such as citrate, including for example sodium citrate/citric acid. Suitable concentrations can be empirically determined, but typically range from 0.005 to 0.05 M, particularly about 0.01 M in an isotonic solution such as saline. In certain instances, pharmaceutical compositions useful in the methods herein are known to those of skill in the art for formulating poloxamer (see, e.g., Published International PCT Application No. WO 94/008596 and other such references and publications described herein).

As used herein, “treatment” refers to ameliorating or reducing symptoms associated with a disease or condition. Treatment means any manner in which the symptoms of a condition, disorder or disease are ameliorated or otherwise beneficially altered. Hence treatment encompasses prophylaxis, therapy and/or cure. Treatment also encompasses any pharmaceutical use of the compositions herein.

As used herein, “treating” a subject having a disease or condition means that a composition or other product provided or described herein is administered to the subject to thereby effect treatment thereof

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “prevention” or “prophylaxis” refers to methods in which the risk of developing a disease or condition is reduced. Prophylaxis includes reduction in the risk of developing a disease or condition and/or a prevention of worsening of symptoms or progression of a disease, or reduction in the risk of worsening of symptoms or progression of a disease.

As used herein an “effective amount” of a compound or composition for treating a particular disease is an amount that is sufficient to ameliorate, or in some manner reduce symptoms to achieve the desired physiological effect. Such amount can be administered as a single dosage or can be administered according to a regimen, whereby it is effective. The effective amount is readily determined by one of skill in the art following routine procedures, and depends upon the particular indication for which the composition is administered.

As used herein, “therapeutically effective amount” or “therapeutically effective dose” refers to an agent, compound, material, or composition containing a compound that is at least sufficient to produce a therapeutic effect. An effective amount is the quantity of a therapeutic agent sufficient to treat, such as prevent, cure ameliorate, arrest or otherwise treat a particular disease or disorder.

As used herein, “disease” or “disorder” refers to a pathological condition in an organism resulting from cause or condition including, but not limited to, infections, acquired conditions, and genetic conditions, and characterized by identifiable symptoms. Diseases and disorders of interest herein include, but are not limited to, any requiring membrane resealing and repair, tissue ischemia and reperfusion injury, decreasing inflammatory disorders, disorders related thrombolysis, and disorders related to hemostasis. For example, diseases and disorders include acute myocardial infarction, acute limb ischemia, shock, acute stroke, heart failure, sickle cell disease, neurodegenerative diseases, macular degeneration, diabetic retinopathy and congestive heart failure.

As used herein, “subject” refers to an animal, particularly human or a veterinary animal, including dogs, cats, pigs, cows, horses and other farm animals, zoo animals and pets. Thus, “patient” or “subject” to be treated includes humans and or non-human animals, including mammals. Mammals include primates, such as humans, chimpanzees, gorillas and monkeys; domesticated animals, such as dogs, horses, cats, pigs, goats, cows; and rodents such as mice, rats, hamsters and gerbils.

As used herein, a “combination” refers to any association between two or among more items. The association can be spatial, such as in a kit, or refer to the use of the two or more items for a common purpose.

As used herein, a “composition” refers to any mixture of two or more products or compounds (e.g., agents, modulators, regulators, etc.). It can be a solution, a suspension, liquid, powder, a paste, aqueous or non-aqueous formulations or any combination thereof.

As used herein, an “article of manufacture” is a product that is made and sold. The term is intended to encompass purified poloxamers contained in articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a “kit” refers to a packaged combination, optionally including reagents and other products and/or components for practicing methods using the elements of the combination. For example, kits containing purified poloxamers provided herein and another item for a purpose including, but not limited to, administration, diagnosis, and assessment of a biological activity or property are provided. Kits optionally include instructions for use.

As used herein, animal includes any animal, such as, but not limited to; primates including humans, gorillas and monkeys; rodents, such as mice and rats; fowl, such as chickens; ruminants, such as goats, cows, deer, sheep; ovine, such as pigs and other animals. Non-human animals exclude humans as the contemplated animal.

As used herein, the singular forms “a,” “an” and “the” include plural referents unless the context clearly dictates otherwise.

As used herein, ranges and amounts can be expressed as “about” or “approximately” a particular value or range. About also includes the exact amount. Hence “about 0.05 mg/mL” means “about 0.05 mg/mL” and also “0.05 mg/mL.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein “retention time” or t_{R}” means the time elapsed between the injection of a sample, such as an LCMF poloxamer 188 sample, onto a reverse phase column for reverse phase high performance liquid chromatography and the peak response by the evaporative light scattering detector. The retention time is longer for more hydrophobic samples compared to less hydrophobic samples.

As used herein “capacity factor” or k′ is determined by the following equation where t₀ is equal to the void time or the time a non retained substance passes through a reverse phase HPLC column (see, Example 7 below):

$${k}^{′} = \frac{{t}_{R} - {t}_{0}}{{t}_{0}} .$$

LCM-containing purified poloxamer 188, such as the poloxamer sold under the trademark FLOCOR®, has a mean retention time (t_{R}) of 9.883 and a k′ of 3.697; whereas the LCMF poloxamer 188 has a mean retention time (t_{R}) of 8.897 and a mean k′ of 3.202 (see Example 7).

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. HEMOSTASIS AND POLOXAMERS

Provided herein are methods for improving hemostasis by administering a sufficient amount of a poloxamer, particularly a poloxamer 188 as described herein, to control, reduce, or modulate hemostatic disruption. Impairment of any step or any aspect of hemostasis including the steps of hemostasis and/or the coagulation cascade, can result in hemostatic dysfunction. Impairment can result from a variety of causes, including but not limited to, genetic diseases of coagulation, injury or trauma, surgery, or administration of thrombolytics or anti-coagulants. Therapeutic intervention at any step or steps or aspects of the process can improve hemostasis. In the methods herein a poloxamer is administered to improve hemostasis by decreasing bleeding or reducing the risk thereof. It is administered at a dosage sufficient to restore any or all of the steps or aspects of hemostasis, and particularly affects the formation or function of fibrin in a blood clot.

1. Hemostasis and Hemostatic Dysfunction

Hemostasis occurs in three steps: vascular spasm, platelet plug formation (primary and secondary hemostasis) and blood coagulation. During vascular spasm, the damaged vessels constrict (vasoconstriction) to reduce the amount of blood flow through the area and prevent blood loss. Vasoconstriction is followed by platelet plug formation whereby platelets are activated and adhere to the damaged endothelium to form a primary hemostatic plug (e.g., a soft platelet plug). This stage is called primary hemostasis. During secondary hemostasis, a fibrin mesh is formed in and around the primary hemostatic plug through the activation of the coagulation cascade. The coagulation cascade is a series of clotting factors that are activated through two pathways (e.g., the intrinsic pathway and the extrinsic pathway) that lead to the formation of a fibrin clot. The third step, blood coagulation, occurs to reinforce the platelet plug and to aid in closing and maintaining the platelet plug on larger wounds. As the fibrin mesh begins to form during secondary hemostasis, the localized blood is transformed from a liquid to a gel-like substance through the activity of clotting factors and procoagulants. The release of Prothrombin, a member of the coagulation cascade, promotes clot formation. This final step forces blood cells and platelets to stay trapped in the wounded area while healing commences.

To prevent excessive growth of blood clots, the body relies on fibrinolysis, the process by which a fibrin clot is dissolved. Fibrinolysis involves the conversion of the inactive precursor, plasminogen, to the active enzyme, plasmin, which digests the fibrin mesh into fragments that are further degraded by other proteases and cleared by the kidney and liver. Tissue plasminogen activator (t-PA), streptokinase and urokinase are agents that convert plasminogen to the active plasmin, to initiate fibrinolysis.

a. Primary Hemostasis

Primary hemostasis is defined as the formation of the primary platelet plug and involves platelets, the blood vessel wall and von Willebrand factor. Undamaged endothelium prevents hemostasis by providing a physical barrier and by secreting products which inhibit platelet adhesion and activation, including nitric oxide and prostaglandin 12 (prostacyclin). Following injury to the vessel wall, the initial event is vasoconstriction, which is a transient, locally-induced phenomenon. Vasoconstriction not only retards extravascular blood loss, but also slows local blood flow, enhancing the adherence of platelets to exposed subendothelial surfaces and the activation of the coagulation process. The formation of the primary platelet plug involves platelet adhesion followed by platelet activation and then aggregation to form a platelet plug.

i. Platelet Adhesion

The first event in primary hemostasis is the adhesion of platelets to exposed subendothelium. In areas of high shear rate (in the microvasculature), this is mediated by von Willebrand factor (vWf), which binds to glycoprotein Ib-IX in the platelet membrane. In areas of low shear rate (e.g. aorta), fibrinogen mediates the binding of platelets to the subendothelium (by attaching to a platelet receptor—the integrin, glycoprotein Ia/IIa).

ii. Platelet Activation

The adhesion of platelets to the vessel wall activates them, causing the platelets to change shape, to activate the collagen receptor on their surface (an integrin receptor called glycoprotein IIb/IIIa) and to undergo the release reaction (release alpha and dense granule constituents). In addition, upon activation, platelets synthesize and release thromboxane A2 (TXA2) and platelet activating factor (PAF), which are potent platelet aggregating agonists and vasoconstrictors.

iii. Platelet Aggregation

TXA2, PAF, ADP and serotonin (ADP and serotonin are released from dense granules) are platelet agonists, causing the activation and recruitment of additional platelets, which bind to the adhered platelets. This activation is enhanced by the generation of thrombin (another platelet agonist), through the coagulation cascade. Platelet aggregation is mediated primarily by fibrinogen (vWf has a secondary role), which binds to glycoprotein IIb/IIIa on adjacent platelets. This aggregation leads to the formation of the primary platelet plug, which must be stabilized by the formation of fibrin.

Platelets also contribute to secondary hemostasis (coagulation cascade) by providing a phospholipid surface (formerly called PF3) and receptors for the binding of coagulation factors.

b. Secondary Hemostasis and the Coagulation Cascade

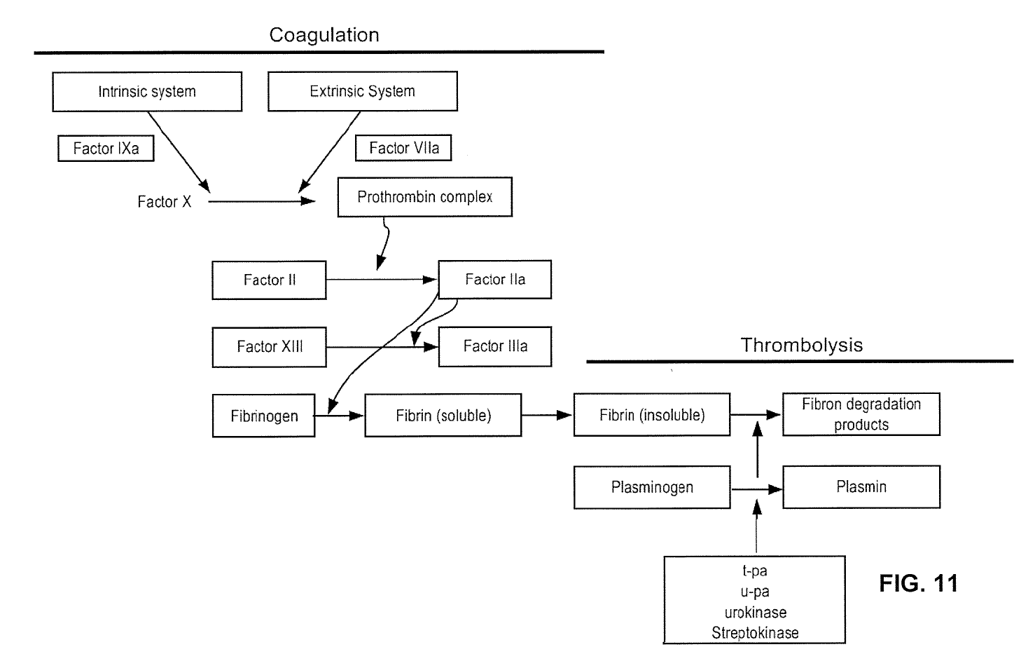

Secondary hemostasis involves plasma coagulation factors, which act in a proteolytic cascade (FIG. 11) to generate soluble fibrin from fibrinogen, which polymerizes to form fibrin strands that strengthen and reinforce the platelet plug (insoluble fibrin). The proteolytic cascade occurs by a series of cleavage reactions, in which a circulating zymogen (inactive enzyme precursor) of a serine protease and its glycoprotein co-factor are activated to become active components that then catalyze the next reaction in the cascade, ultimately resulting in cross-linked fibrin. Coagulation factors, which include the serine proteases and co-factors, are generally indicated by Roman numerals (e.g., factor II). The coagulation cascade requires the serine proteases, (e.g., factors II, VII, IX, X, XI, and XII) cofactors (factors V and VIII), calcium, and platelets. Platelets provide a source of phospholipid [PF3] and a binding surface upon which the coagulation cascade proceeds.

The coagulation cascade is classically divided into three pathways: the extrinsic (or Tissue Factor) pathway, the intrinsic (or contact activation) pathway and the common pathway. The intrinsic and extrinsic pathways occur in parallel, and converge at factor X to form the common pathway, which results in the formation of the Fibrin clot (Mann et al. (1990) Blood, 76(1):1-16).

The coagulation cascade is separated into 3 pathways: intrinsic, extrinsic and common pathways (FIG. 11). The extrinsic pathway involves the tissue factor and factor VII complex, which activates factor X. The intrinsic pathway involves high-molecular weight kininogen, prekallikrein, and factors XII, XI, IX and VIII. Factor VIII acts as a cofactor (with calcium and platelet phospholipid) for the factor IX-mediated activation of factor X. The extrinsic and intrinsic pathways converge at the activation of factor X. The common pathway involves the factor X-mediated generation of thrombin from prothrombin (facilitated by factor V, calcium and platelet phospholipid), with the ultimate production of fibrin from fibrinogen.

2. Poloxamers and Hemostasis

Poloxamers, such as those described herein, are administered in a dosage sufficient to improve hemostasis. They can improve clotting or reduce bleeding or other such parameter by virtue of effects on the kinetics of hemostasis.

The kinetics of hemostasis can be modulated by the presence, or abundance, of intrinsic and extrinsic components of the native hemostatic system. For example, increasing concentrations of intrinsic components such as calcium ions, fibrinogen, phospholipid, prothrombin, and platelets, which are directly involved in promoting the hemostasis pathway can increase hemostatic kinetics by increasing rates of clot formation. Treatment with anti-coagulation factors and/or thrombolytic agents, such as, but not limited to, tissue plasminogen activator (t-PA), anistreplase, streptokinase, urokinase, plasmin, heparin, low molecular weight heparin, warfarin, Factor Xₐ inhibitors, cyclooxygenase inhibitors, thromboxane inhibitors, ADP re-uptake inhibitors or antagonists, phosphodiesterase inhibitors, glycoprotein IIb/IIa antagonists, other anti-platelet agents, and other anticoagulant agents can act to slow or prevent hemostasis.

Poloxamers are nonionic triblock copolymer surfactants composed of a central hydrophobic chain of polyoxypropylene (poly(propylene oxide)) flanked by two hydrophilic chains of polyoxyethylene (poly(ethylene oxide)), and are further described below. Certain poloxamers have been used to treat several diseases or conditions associated with an increased risk for blood clotting (thrombotic disorders) or as an adjunct to thrombolytic therapy. The activities related to treatments associated with thrombotic disorders have been described, for example, in U.S. Pat. Nos. 4,801,452, 4,837,014, 4,873,083, 4,879,109, 4,897,263, 4,937,070, 4,997,644, 5,017,370, 5,028,599, 5,030,448, 5,032,394, 5,039,520, 5,041,288, 5,047,236, 5,064,643, 5,071,649, 5,078,995, 5,080,894, 5,089,260, RE 36,665 (Reissue of 5,523,492), 5,605,687, 5,696,298, 6,359,014, and 6,747,064, 8,372,387, 8,580,245, U.S. Patent Publication Nos. 2011/0044935, 2011/0212047, 2013/0177524. None of these describe treating hemostatic dysfunction involving bleeding, to improve hemostasis such as be reducing or preventing bleeding, as described herein.

In some of these prior art therapies, poloxamers are reported to act, at least in part, as a lubricant, facilitating blood flow by blocking the interactions of adhesive hydrophobic proteins circulating in the blood, such as fibrinogen, and/or on the surface of components of the blood, such as red blood cells, with other components of the blood or damaged membranes. For example, poloxamers can reduce the aggregation of red blood cells through reduced cell surface resistance. Poloxamers also are reported to contribute to fibrinolysis, for example, by facilitating flow through an occlusive thrombus thus increasing the delivery of a thrombolytic agent (Hunter et al., (1990) Fibrinolysis. 4:117-123) or by altering the fibrin polymer structure such that they are more susceptible to degradation by thrombolytic agents, such as t-PA. Poloxamer 188 (P188) and compositions containing such poloxamers, described in further detail in the following section, have been used in rheologic, anti-thrombotic and cytoprotective applications, but not to treat hemostatic dysfunction manifested as described herein, including by bleeding, to improve hemostasis, such as by reducing side effects of thrombolytic therapy. P188 has been shown to alter fibrin polymerization in plasma and in whole blood, resulting in increased fibrin fiber size, increased turbidity and permeability of the fibrin network, and enhanced fibrinolysis (Carr et al., (1991) Thromb Haemost. 66(5):565-568; van Gelder et al., (1993) Thromb Res. 71(5):361-376). P188 also can accelerate fibrin assembly kinetics, reducing the lag phase for initiation of fibrin network assembly (Can et al., (1991) Thromb Haemost. 66(5):565-568).

Thus, while applications for poloxamer 188 (P188 or P188), reported in the art, have involved its use in thrombolytic/fibrinolytic therapies, the methods provided herein employ P188 to improve hemostasis by reducing hemostatic dysfunction as described herein. In the methods provided herein, P188, including LCMF P188, is administered at a dosage that results in improved hemostasis in applications in which reducing or stopping of bleeding or reduction in risk of bleeding, or improving clot formation, and otherwise modulating, particularly reversing or decreasing, the undesirable side-effects of anti-coagulant and thrombolytic therapies, are needed.

### C. POLOXAMERS AND PURIFIED POLOXAMERS

Provided herein are methods and uses of a poloxamer, and in particular a poloxamer 188 (P188), such as a purified P188, including LCMF poloxamer 188, for treating hemostatic dysfunction as described herein. Poloxamers are a family of synthetic, linear, triblock copolymers composed of a core of repeating units of poly(oxypropylene) (PO), flanked by chains of repeating units of (poly)oxyethylene (EU). All poloxamers are defined by this EO-PO-EO structural motif. Specific poloxamers (e.g. P188) are further defined by the number of repeating EO and PO units, which provide specific poloxamers with different chemical and physical characteristics, as well as unique pharmacodynamic properties.

Certain polyoxyethylene/polyoxypropylene copolymers, including P188, have been found to have beneficial biological effects on several disorders when administered to a human or animal. These activities have been described in U.S. Pat. Nos. 4,801,452, 4,837,014, 4,873,083, 4,879,109, 4,897,263, 4,937,070, 4,997,644, 5,017,370, 5,028,599, 5,030,448, 5,032,394, 5,039,520, 5,041,288, 5,047,236, 5,064,643, 5,071,649, 5,078,995, 5,080,894, 5,089,260, RE 36,665 (Reissue of 5,523,492), 5,605,687, 5,696,298 6,359,014, 6,747,064, 8,372,387, 8,580,245, U.S. Patent Publication Nos. 2011/0044935, 2011/0212047, 2013/0177524, and International Applications PCT/US2005/034790, PCT/US2005/037157 and PCT/US2006/006862. Among the activities of poloxamers, such as P188, that make them useful as therapeutic agents is their ability to incorporate into cellular membranes, and thereby repair damaged cell membranes.

Poloxamers for use in the methods provided herein include POP/POE block copolymers having the following formula:

HO(C₂H₄O)_{a′}—(C₃H₆O)_{b}—(C₂H₄O)ₐH,

where “a′” and “a” can be the same or different and each is an integer such that the hydrophile portion represented by (C₂H₄O) constitutes approximately 50% to 95% by weight of the compound, such as 60% to 90%, for example 70% to 90%, by weight of the compound; and the “b” is an integer such that the hydrophobe represented by (C₃H₆O) has a molecular weight of approximately 950 to 4,000 Da, such as 1,200 to 3,500 Da. For example, the hydrophobe has a molecular weight of 1,200 to 2,300 Da, such as generally 1,500 to 2,100 Da. The average molecular weight of the copolymer is 5,000 to 15,000 Da, such as 5,000 to 12,000 Da, for example 5000 and 9000 Da.

In certain instances, b is an integer of from about 15 to about 70, such as from about 15 to about 60, or from about 15 to about 30, or any of the numbers in between. In some instances, b is about 15, 16, 17, 18, 19, 20, 21, 22, 23, 24, 25, 26, 27, 28, 29, or 30. In certain aspects, the integers for the flanking units with the subscript “a” and “a” can differ or are the same values. In some instances, a or a′ is an integer of about 45 to about 910, such as 90, 100, 200, 300, 400, 500, 600, 700, 800, or 900. In some other instances, a or a′ is an integer from about 10 to about 215, such as 10, 20, 30, 40, 50, 60, 70, 80, 100, 125, 150, 175, 200 or 215. In still other instances, a or a′ is about 30, 31, 32, 33, 34, 35, 36, 37, 38, 39, 40, 41, 42, 43, 44, 45, 46, 47, 48, 49, 50, 51, 52, 53, 54, 55, 56, 57,58, 59, 60, 61, 62, 63, 64, 65, 66, 67, 68, 69, or 70. A skilled artisan will appreciate that these values are average values. That is, the values for a′, a and b represent an average, and generally the polymeric molecules are a distribution or population of molecules and therefore the actual values of a, a′ and b within the population will constitute a range of values.

The nomenclature of the poloxamer relates to the composition of the various polymer members. The first two digits of a poloxamer number, multiplied by 100, gives the approximate molecular weight of the hydrophobe. The last digit, times 10, gives the approximate weight percent of the hydrophile (polyoxyethylene) content of the surfactant. For example, poloxamer 407 describes a polymer containing a polyoxypropylene hydrophobe of about 4,000 Da with the polyoxyethylene hydrophile comprising about 70% of the total molecular weight. Poloxamer 188 (P188) has a hydrophobe with a molecular weight of about 1,800 Da and has a hydrophile that is about 80% of the total molecular weight of the copolymer.

Poloxamers are sold and reference under trade names including, but not limited to, ADEKA NOL, Synperonic™, Pluronic® and Lutrol®. Exemplary poloxamers include, but are not limited to, poloxamer 188 (P188; sold under the trademarks Pluronic® F-68, Kolliphor® P188, RheothRx and Flocor™; 80% POE), poloxamer 407 (P407; sold under the trademark Lutrol F-127, Kolliphor® P188, Pluronic® F-127; 70% POE), poloxamer 237 (P237; sold under the trademark Pluronic® F87, Kolliphor® P 237; 70% POE), and poloxamer 338 (P338; sold under the trademark Kolliphor® P 338, Pluronic® F-108; 80% POE).

Poloxamers, including P188, for use in the methods herein include purified preparations of a poloxamer. Poloxamers are sold and referred to under trade names and trademarks, in including, but not limited to, ADEKA NOL, Synperonic™, Pluronic® and Lutrol®. Exemplary poloxamers include, but are not limited to, poloxamer 188 (P188; sold under the trademarks Pluronic® F-68, Kolliphor® P188, 80% POE), poloxamer 407 (P407; sold under the trademark Lutrol F-127, Kolliphor® P188, Pluronic® F-127; 70% POE), poloxamer 237 (P237; sold under the trademark Pluronic® F87, Kolliphor® P 237; 70% POE), poloxamer 338 (P338; sold under the trademark Kolliphor® P 338, Pluronic® F-108; 80% POE) and poloxamer 331 (Pluronic® L101; 10% POE).

Hence, non-purified P188 is commercially available or known under various names as described above. While the discussion below references using the methods herein to produce a more homogenous (LCMF) poloxamer 188, methods herein can be used to produce more homogenous preparations of any of the known poloxamers.

Poloxamers can be synthesized using standard polymer synthesis techniques. For example, poloxamers are formed by ethylene oxide-propylene oxide condensation using standard techniques know to those of ordinary skill in the art (see, e.g., U.S. Pat. Nos. RE 36,665, RE 37,285, RE 38,558, 6,747,064, 6,761,824 and 6,977,045; see also Reeve, L. E., The Poloxamers: Their Chemistry and Medical Applications, in Handbook of Biodegradable Polymers, Domb, A. J. et al. (eds.), Hardwood Academic Publishers, 1997). Poloxamers can be synthesized by sequential addition of POP and POE monomers in the presence of an alkaline catalyst, such as sodium or potassium hydroxide (See, e.g., Schmolka, J. Am. Oil Chem. Soc. 54 (1977) 110-116). The reaction is initiated by polymerization of the POP block followed by the growth of POE chains at both ends of the POP block. Methods of synthesizing polymers also are described in U.S. Pat. No. 5,696,298.

1. Poloxamer 188 (P188)

Exemplary of poloxamers in the methods and uses provided herein is poloxamer 188 (P188) and purified preparations thereof. A poloxamer 188 (P188) copolymer has the following chemical formula:

HO(CH₂CH₂O)_{a′}—[CH(CH₃)CH₂O]_{b}—(CH₂CH₂O)ₐH,

where the hydrophobe represented by (C₃H₆O) has a molecular weight of approximately 1,750 Daltons and the poloxamer 188 has an average molecular weight of 7,680 to 9,510 Da, such as generally approximately 8,400-8,800 Daltons. The polyoxyethylene-polyoxypropylene-polyoxyethylene weight ratio of is approximately 4:2:4. According to specifications, P188 has a weight percent of oxyethylene of 81.8±1.9%, and an unsaturation level of 0.026±0.008 mEq/g.

Various poloxamers, and in particular P188, are used for treatment of diseases and conditions in which resistance to blood flow is pathologically increased by injury due to the presence of adhesive hydrophobic proteins or damaged membranes. This adhesion is produced by pathological hydrophobic interactions and does not require the interaction of specific ligands with their receptors. Such proteins and/or damaged membranes increase resistance in the microvasculature by increasing friction and reducing the effective radius of the blood vessel. For example, it is believed that poloxamer 188 acts as a lubricant to increase blood flow through damaged tissues. Advantageously, this blocks adhesion of hydrophobic surfaces to one another and thereby reduces friction and increases flow.

Poloxamer 188 binds to hydrophobic areas developed on injured cells and denatured proteins thereby restoring hydration lattices. Such binding facilitates sealing of damaged membranes and aborts the cascade of inflammatory mediators that could destroy the cell. This polymer also inhibits hydrophobic adhesive interactions that cause deleterious aggregation of formed elements in the blood. P188's anti-adhesive and anti-inflammatory effects are exhibited by enhancing blood flow in damaged tissue by reducing friction, preventing adhesion and aggregation of formed elements in the blood, maintaining the deformability of red blood cells, non-adhesiveness of platelets and granulocytes, the normal viscosity of blood, reducing apoptosis, and by multiple markers of inflammation including VEGF, various chemokines, interleukins, and chemokines.

2. Molecular Diversity of Poloxamer 188

Commercially available poloxamer 188 preparations are stated to have a molecular weight of approximately 8400 Daltons. Such poloxamer 188, however, is composed of molecules having a molecular weight from less than 3000 Daltons to over 20,000 Daltons. The molecular diversity and distribution of molecules of commercial poloxamer 188 can be seen in the broad primary and secondary peaks detected using gel permeation chromatography (see, e.g., International PCT published application No. WO 94/08596).

The diversity in structure means that there is a diversity in biological activity. For example, the optimal rheologic, cytoprotective, anti-adhesive and antithrombotic effects are observed with molecules of P188 that are approximately 8,400 to 9,400 daltons. Such components can be identified as the main or predominant component in a poloxamer preparation using methods that separate components based on size, such as gel permeation chromatography (GPC). The distribution of components, however, also typically show a smaller fraction of low molecular weight (LMW, i.e. generally below 4,500 Daltons) or high molecular weight (HMW, i.e. generally above 13,000 Daltons,) components. P188 components above 15,000 and below 4,500 Daltons are less effective rheologic or cytoprotective agents and exhibit unwanted side effects. The other substances or components in a poloxamer preparation, such as a P188 preparation, originate from two different sources, synthesis and degradation.

A primary mechanism contributing to the molecular diversity is the process by which poloxamers are synthesized. During the typical manufacturing process, the first step is the formation of the POP blocks. These are formed by reacting a propylene glycol initiator with propylene oxide monomer. Subsequently, ethylene oxide monomer is added to both ends forming the block copolymer. The synthesis of poloxamers can result in a variation in the rates of polymerization during the steps of building the PO core and EO terminal ends.

During the synthesis of the POP, two different reaction mechanisms limit POP chain growth and result in unintended diblock polymers. These substances are typically of lower molecular weight (relative to the polymeric distribution of P188). In one mechanism, unsaturation is formed directly from propylene oxide by reacting with an alkali catalyst. The base catalyzes the rearrangement of the propylene oxide to an allyl alcohol, which then initiates a mono functional chain with terminal unsaturation. These types of side reactions will produce low molecular weight (LMW) substances throughout the time of the reaction. On gel permeation chromatography (GPC), the distribution of these impurities are located in the main peak as well as in the LMW shoulder. In a second mechanism, the abstraction of a hydrogen atom, located six carbon atoms away, by the negative oxygen atom in a growing polymer chain can terminate and transfer the chain, producing an allyl end group. These back-biting reactions are predominant with high molecular weight (HMW) POP blocks. The distribution of these substances is mostly in the LMW shoulder.

In addition, high molecular weight substances (relative to the polymeric distribution of P188) can be formed due to inadequate cleaning of the polymerization reactor between batches of poloxamer 188 during a typical commercial manufacturing campaign. If the reactor is not completely cleaned to remove residual product after manufacturing a typical batch of poloxamer, such as P188, the residual product will act as an initiator in the subsequent batch and form a “dimer like” poloxamer molecule. This substance is of higher molecular weight and would be part of the polymeric distribution observed on GPC as the HMW shoulder.

The degradation pathways for poloxamers include peroxidation leading to low molecular aldehydes and acids and thermal degradation leading to LMW polyethylene glycols. Oxidative degradation is the primary degradation pathway affecting stability of poloxamers. This process generates structural changes to the polymer chain and generates peroxides and carbonyls. Peroxides are transient in nature and quickly combine with butylated hydroxytoluene (BHT), which is typically added to commercial preparations as an antioxidant. Thermal degradation is another pathway that produces other substances. Glycols of various chain lengths are major degradation products of thermal degradation. Forced thermal degradation studies have shown that ethylene glycol, propylene glycol, diethylene glycol and triethylene glycol are formed.

Thus, specific poloxamers are composed of multiple chemical entities that have the EO-PO-EO structural motif, but vary in the number of repeating EO and PO units. Various truncated polymers with an EO-PO motif and a variety of other substances can form as a result of side reactions occurring during synthesis of the intended poloxamer compound. These other substances can be present and found within the overall poloxamer distribution. The result is material that is non-uniform (i.e. material that is polydisperse).

For example, due to the synthesis of P188, there can be variation in the rates of polymerization during the steps of building the PO core and EO terminal ends. Thus, most non-purified forms of P188 contain a bell-shaped distribution of polymer species, which vary primarily in overall chain length. In addition, various low molecular weight (LMW) components (e.g. glycols and truncated polymers) formed by incomplete polymerization, and high molecular weight (HMW) components (e.g. dimerized polymers) can be present. Typically, characterization of P188 by gel permeation chromatography (GPC) identifies a main peak of P188 with “shoulder” peaks representing the unintended LMW and HMW components (Emanuele and Balasubramaniam (2014) Drugs R D, 14:73-83). For example, the preparation of P188 that is available from BASF (Parsippany, N.J.) has a published structure that is characterized by a hydrophobic block with a molecular weight of approximately 1,750 daltons (Da), POE blocks making up 80% of the polymer by weight, and a total molecular weight of approximately 8,400 Da. The actual compound is composed of the intended POE-POP-POE copolymer, but also contains other molecules which range from a molecular weight of less than 1,000 Da to over 30,000 Da. The molecular diversity and distribution of molecules of commercial poloxamer 188 is illustrated by broad primary and secondary peaks detected using gel permeation chromatography.

The diversity of molecules present in the non-purified poloxamer preparations, including commercially available poloxamers, can result in diverse biological activities. Many of the observed biological activities are undesired or/and can result in unwanted side effects that limit the therapeutic efficacy of poloxamers as drugs. Complement activation, phagocyte migration paralysis, and cytotoxicity observed upon administration of artificial blood preparations have been attributed in part to impurities in the poloxamer 188 component of those preparations. In addition, infusion of poloxamer 188 was shown to result in elevated creatinine, indicating kidney damage, and increased organ weights (kidney) in toxicological animal studies. Histologic evaluation of the kidney demonstrated a dose related cytoplasmic vacuolation of the proximal tubular epithelial cells.

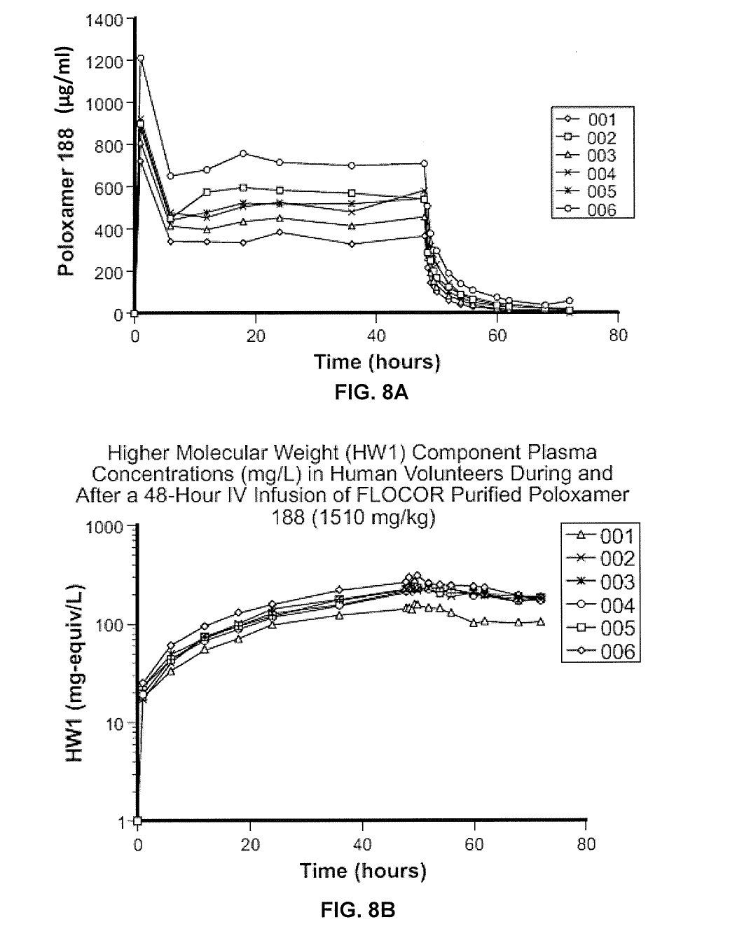

Poloxamer 188 (see, e.g., Grindel et al. (2002) Journal of Pharmaceutical Sciences, 90:1936-1947 (Grindel et al. 2002a) or Grindel et al. (2002) Biopharmaceutics & Drug Disposition, 23:87-103 (Grindel et al. 2002b)), which is purified to remove lower molecular weight components, contains components that, when administered to a subject, exhibit different pharmacokinetic profiles. The main component exhibits a half-life (t_{1/2}) in plasma of about 7 hours and a higher molecular weight component (i.e. the longer retention time species) exhibits about a 10-fold or more increase in half-life with a t_{1/2} of approximately 70 hours or more and, thus, a substantially longer plasma residence time with slower clearance from the circulation than the main component. This is demonstrated herein (see, FIG. 8A and FIG. 8B).

a. Low Molecular Weight Components

Substances in poloxamer 188 that are toxic to kidneys have been identified as being of lower molecular weights than the main components. Studies on the therapeutic potential of P188 led to the discontinuance of the poloxamer available under the trademark RheothRx® for therapeutic applications in part due to an acute renal dysfunction observed during clinical trial evaluation as evidenced by elevated serum creatinine. It was found that these effects were due to the presence of various low molecular weight (LMW) substances that formed during the synthesis process (Emanuele and Balasubramanian (2014) Drugs R D, 14:73-83). The LMW substances were accumulated by the proximal tubule epithelial cells in the kidney.

The molecular weight of the LMW substances can range from a few hundred Da to a few thousand Da. The complex nature of these impurities with wide solubility characteristics make it difficult to selectively remove them from the parent molecules. Conventional purification processes such as distillation, crystallization, ultrafiltration, and the like, do not effectively separate the low molecular weight (LMW) substances from the main component. Use of chromatographic techniques for purification such as preparative GPC are expensive and practically difficult to scale-up. Fine-tuning mixed solvent systems to differentially solubilize and remove various substances is also challenging and requires the use of large amounts of solvents that are costly to recycle.

Supercritical fluid chromatography that reduces the level of these low molecular weight substances present in P188 has been reported (see, e.g., U.S. Pat. No. 5,567,859). Supercritical fluid extraction was performed using carbon dioxide to purify the copolymers to reduce the polydispersity to less than 1.17. The method, however, does not sufficiently remove or reduce LMW components and, as shown herein, other components.

As described in more detail below, the methods provided herein produces poloxamer preparations that are substantially free of these LMW components. For example, purified P188 reduced in LMW components have less than about 5%, 4%, 3%, 2% or 1% LMW components. Thus, the poloxamer preparations provided herein, and in particular P188 poloxamer preparations, generally exhibit reduced toxicity and do not result in elevated creatinine levels when administered. In addition, as described the resulting P188 poloxamer preparation has other advantageous properties, including a reduction of long circulating material upon administration.

b. Components Resulting in Long Circulating Half-Life

A component in P188 has been identified that is or gives rise to a material in the plasma or blood with a longer circulating half-life compared to the main or predominant poloxamer species. This material with the longer circulating half-life is observed in non-clinical and clinical studies. Analysis of plasma obtained following intravenous administration of purified P188 by high performance liquid chromatography-gel permeation chromatography (HPLC-GPC) shows two distinct peaks in the circulation (Grindel et al. (2002) Journal of Pharmaceutical Sciences, 90:1936-1947 (Grindel et al. 2002a) or Grindel et al. (2002) Biopharmaceutics & Drug Disposition, 23:87-103 (Grindel et al. 2002b)). There is a main peak with average peak molecular weight of about 8,600 daltons and a smaller peak with an average molecular weight of about 16,000 daltons. The two peaks exhibit distinctly different pharmacokinetic profiles with the higher molecular weight peak exhibiting a distinctly longer plasma residence time with slower clearance from the circulation (see FIG. 8A and FIG. 8B). Similar observations were reported in rats and dogs. A similar longer circulating component is observed with native or unpurified poloxamer 188 (see International PCT Published Application No. WO 94/008596)

For example, as shown in FIG. 8A, following administration of a purified P188 intravenously to healthy volunteers as a loading dose of 100 mg/kg/hr for one hour followed by a maintenance dose of 30 mg/kg/hr for 47 hours, the main or predominant peak reached a mean maximum concentration (Cmax) of 0.9 mg/mL by the end of the one hour loading infusion. A mean steady state concentration (Css) of 0.5 mg/mL was achieved essentially coincident with the start of the maintenance infusion. With the discontinuation of the maintenance infusion, plasma concentrations declined rapidly with an elimination half-life (t½) of about 7 hours. As shown in FIG. 8B, a HMW component was identified that exhibited a Cmax of 0.2 mg/mL, which was not attained until the end of the maintenance infusion. Steady state was not attained as the concentration continued to accumulate during infusion. Following discontinuation of the maintenance infusion, plasma levels of the high molecular weight peak declined slowly such that plasma levels had only declined by about 33% during the 24 hour post-infusion monitoring period. This elimination rate is approximately 1/10 that of the main peak and the t½ is approximately 70 hours. See, also Grindel et al. (2002) Journal of Pharmaceutical Sciences, 90:1936-1947 (Grindel et al. 2002a) and Grindel et al. (2002) Biopharmaceutics & Drug Disposition, 23:87-103 (Grindel et al. 2002b)). The longer circulating material (or longer retention time material) is identified in the HMW fraction of the P188 distribution (Grindel et al. 2002a). This HMW component was determined to be approximately 16,000 Da as identified by MALDI-TOF mass spectrometry with a fragmentation pattern consistent with a block copolymer (Grindel et al. 2002a).

Since the rheologic, cytoprotective, anti-adhesive and antithrombotic effects of P188 are optimal within the predominant or main copolymers of the distribution, which are approximately 8,400 to 9400 Daltons and have a half-life of about 7 hours, the presence of other components that exhibit a long circulating half-life is not desirable. For example, among the desired activities of P188 is its rheologic effect to reduce blood viscosity and inhibit red blood cell (RBC) aggregation, which account for its ability to improve blood flow in damaged tissues. In contrast, higher molecular weight poloxamers such as P338 (also called Pluronic® F108) and P308 (Pluronic® F98), increase blood viscosity and RBC aggregation (Armstrong et al. (2001) Biorheology, 38:239-247). This is the opposite effect of P188 and indicates that higher molecular weight poloxamer species may have undesirable biological effects.

As described in more detail below, provided are poloxamer preparations that are substantially reduced in the component that is or gives rise to a long circulating material, i.e. they are long circulating material free (LCMF). Also provided are exemplary methods (see, Example 1) for production of LCMF poloxamer. Thus, the LCMF poloxamer preparations provided herein, and in particular LCMF P188 poloxamer preparations, exhibit a more uniform pharmacokinetic profile, and thus a more consistent therapeutic effect. The LCMF poloxamer is described in more detail in the following section.

3. Long Circulating Material Free (LCMF) Poloxamer

For the methods provided herein, the poloxamer can be a long circulating material free (LCMF) P188 that is a purified P188 that has a polydispersity value less than 1.07; has no more than 1.5% of low molecular weight (LMW) components less than 4,500 daltons; no more than 1.5% high molecular weight components greater than 13,000 daltons; a half-life of all components in the distribution of the copolymer that, when administered to a subject, is no more than 5.0-fold longer half-life in the blood or plasma than the half-life of the main component in the distribution of the copolymer. Methods for treating hemostatic dysfunction by administering the LCMF P188 are provided.

The LCMF Poloxamer 188, when administered, does not give rise to a component that has a significantly longer half-life than the main component. The LCMF P188 has the following chemical formula:

HO(CH₂CH₂O)_{a′}—[CH(CH₃)CH₂O]_{b}—(CH₂CH₂O)ₐH

where a′ and a can be the same or different and each is an integer such that the hydrophile portion represented by (C₂H₄O) (i.e., the polyoxyethylene portion of the copolymer) constitutes approximately 60% to 90%, such as approximately 80% or 81%; and b is an integer such that the hydrophobe represented by (C₃H₆O) has a molecular weight of approximately 1,300 to 2,300 Da, such as approximately 1,750 Da; and the average total molecular weight of the compound is approximately 7,680 to 9,510 Da, such as generally 8,400-8,800 Da, for example about or at 8,400 Da, where the copolymer has been purified to remove impurities, including low molecular weight impurities or other impurities, so that the polydispersity value is less than 1.07

Studies have demonstrated that the main peak component of a purified P188 preparation, when administered to a human subject, has a half-life (t_{1/2}) in plasma of about 7 hours (Grindel et al. (2002) Journal of Pharmaceutical Sciences, 90:1936-1947 (Grindel et al. 2002a) or Grindel et al. (2002) Biopharmaceutics & Drug Disposition, 23:87-103 (Grindel et al. 2002b)). The purified poloxamer also resulted in a longer circulating material containing higher molecular weight components that have an average molecular weight of about 16,000 Daltons, which exhibit about a 10-fold or more increase in half-life with a t_{1/2} of approximately 70 hours.

In contrast to the purified P188 characterized in the studies of Grindel et al., (2002a and 2002b), the purified poloxamer, designated LCMF P188, is one in which all components of the polymeric distribution, when administered to a subject, clear from the circulation at approximately the same rate. Thus, the LCMF P188 is different from prior LCM containing p188 poloxamers. Like LCM containing poloxamers, LCMF poloxamer contains a substantially polydisperse composition of less than 1.07, and generally less than 1.05 or 1.03, but where the half-life in the blood or plasma of any components in the distribution of the copolymer, when administered to a human subject, is no more than 5.0-fold longer than the half-life of the main component in the distribution of the copolymer, and generally no more than 4.0-fold, 3.0-fold, 2.0-fold, 1.5-fold more longer. Typically, the LCMF does not contain any component that exhibits a half-life in the blood or plasma, when administered to a subject, that is substantially more than or is more than the main component in the distribution of the copolymer.

In some examples, the half-life in the blood or plasma of all components in the LCMF poloxamer, when administered to a human subject, is such that no component has a half-life that is more than 30 hours, and generally is no more than 25 hours, 20 hours, 15 hours, 10 hours, 9 hours, 8 hours or 7 hours.

Without being bound by theory, higher molecular weight components of the poloxamer polymeric distribution, such as those greater than 13,000 Daltons could account for the longer circulating half-life material. The rate of glomerular filtration of uncharged molecules like poloxamer 188 and purified poloxamer 188 is highly dependent upon molecular size. This is observed for components of the poloxamer 188 polymeric distribution with molecular weights greater than 5,000 Daltons since, the rate of glomerular filtration becomes increasingly restricted above that size threshold. (Chang et al., (1975) Biophysic. J. 15:887-906) Accordingly, the higher molecular weight components of the poloxamer 188 polymeric distribution (such as those greater than 13,000 Daltons would be more likely to be cleared from the circulation. at a slower rate than those of smaller size.

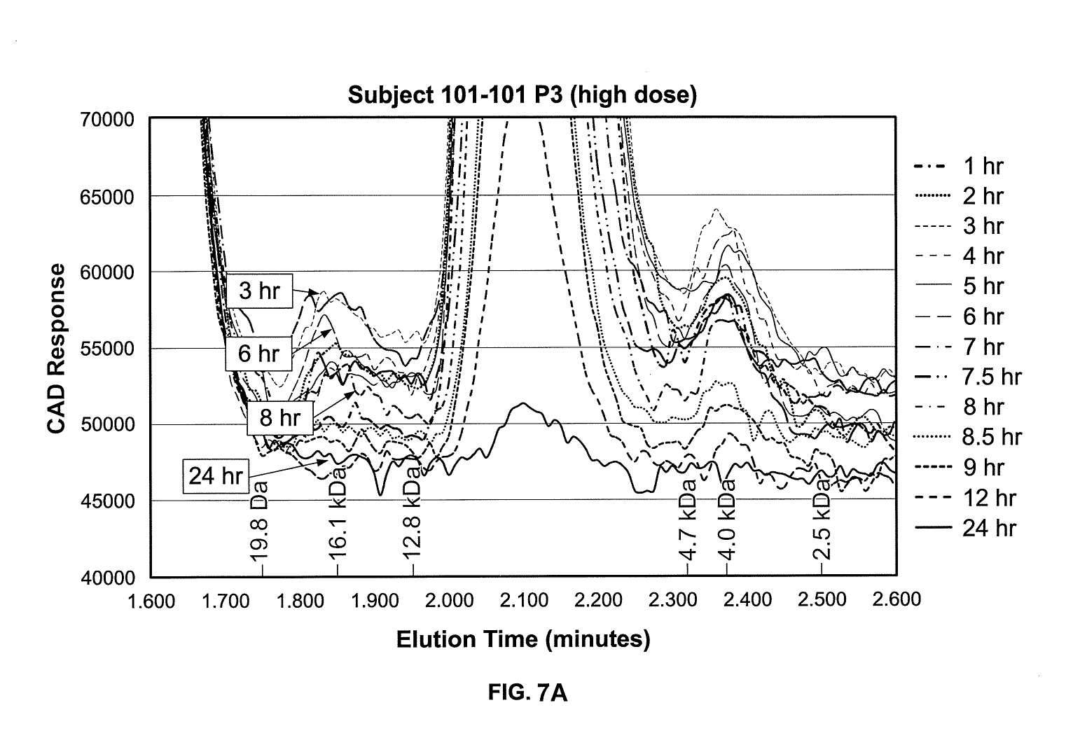

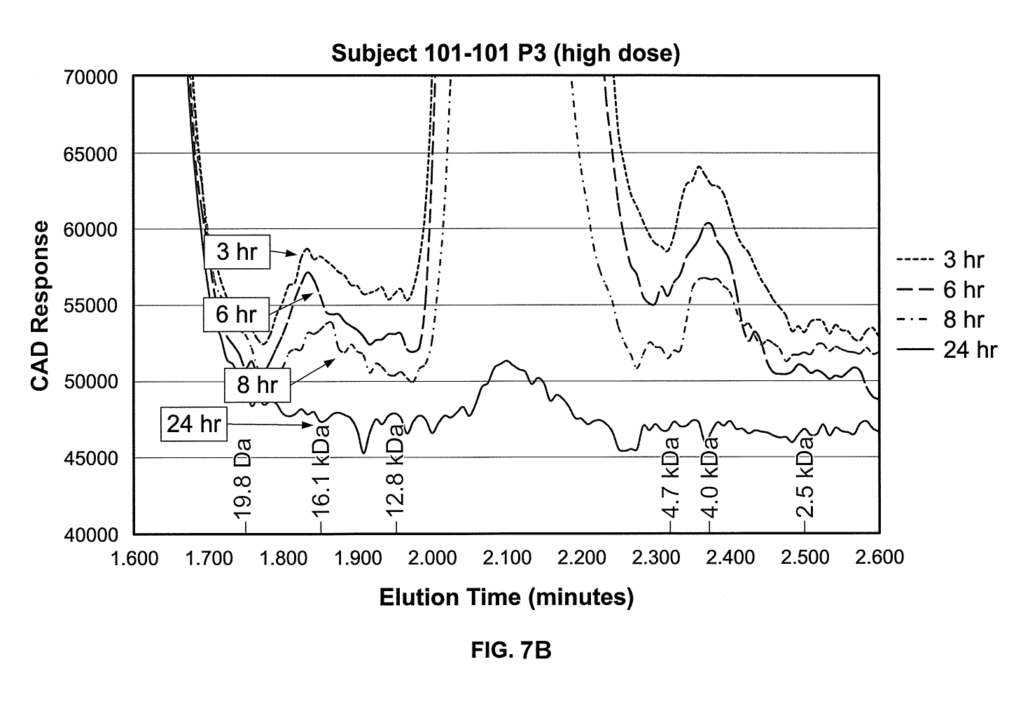

For the LCMF preparations, however, the presence of HMW components in the distribution do not result in a longer circulating species. For example, HMW impurities greater than 13,000 Daltons in an LCMF preparation, generally constitute no more than 1.5% by weight of the total component. When the LCMF preparation is administered to a subject, these HMW impurities do not result in a circulating half-life that is more than 5.0-fold longer than the half-life of the main component in the distribution, and generally no more than 4.0-fold, 3.0-fold, 2.0-fold, 1.5-fold more longer. When the LCMF preparation is administered to a subject, they do not result in any component with a circulating half-life that is substantially more than or is more than the main component in the distribution (see, e.g., FIGS. 7A and 7B).

In the LCMF preparation, the HMW components can be either increased or decreased compared to other existing purified P188 preparations. For example, an LCMF poloxamer provided herein includes P188 poloxamers in which there are no more than 1.3% high molecular weight components greater than 13,000 daltons, such as no more than 1.2%, 1.1%, 1.0% or less. In particular examples provided herein, an LCMF poloxamer provided herein includes P188 poloxamers in which there are less than 1.0% by weight high molecular weight components greater than 13,000 daltons, and generally less than 0.9%, 0.8%, 0.7%, 0.6%, 0.5% or less.

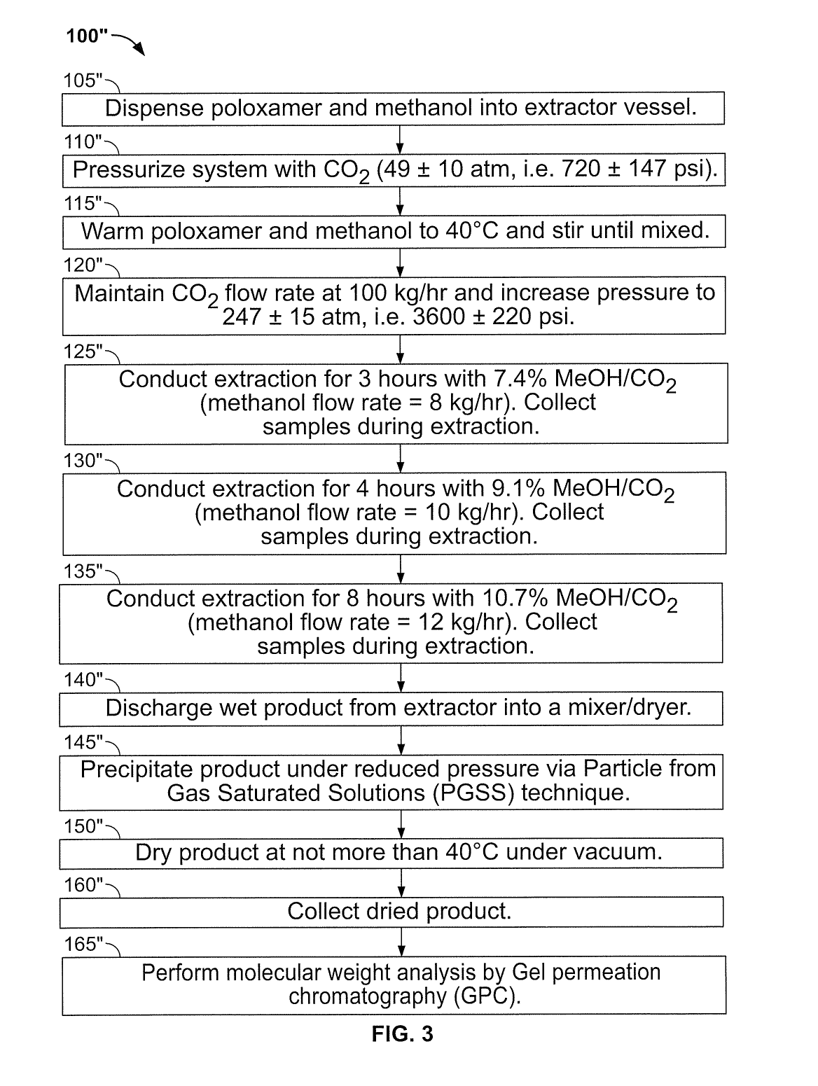

The LCMF poloxamer provided herein can be prepared by methods as described herein below (see section 4, below, and see, e.g., FIG. 3). In view of the description and exemplification of the properties of the LCMF poloxamer, those of skill in the art can envision other methods for producing an LCMF poloxamer. For example, an LCMF poloxamer provided herein is made by a method that includes:

a) introducing a poloxamer solution into an extractor vessel, where the poloxamer is dissolved in a first alkanol to form a solution;

b) contacting the poloxamer solution with an extraction solvent comprising a second alkanol and supercritical carbon dioxide under a temperature and pressure to maintain the supercritical carbon dioxide for a first defined period, wherein:
the temperature is above the critical temperature of carbon dioxide but can typically range between 35° C.-45° C.;
the pressure is 220 bars to 280 bars; and
the alkanol is provided at an alkanol concentration that is 7% to 8% by weight of the total extraction solvent; and

c) increasing the concentration of the second alkanol in step b) in the extraction solvent a plurality of times in gradient steps over time of the extraction method, wherein:

each plurality of times occurs for a further defined period; and

in each successive step, the alkanol concentration is increased 1-2% compared to the previous concentration of the second alkanol; and

d) removing the extraction solvent from the extractor vessel to thereby remove the extracted material from the raffinate poloxamer preparation.

4. Extraction Method for Purifying Poloxamers

Any method known to a skilled artisan can be used to purify a poloxamer. In particular, supercritical methods can be employed. A supercritical extraction permits control of the solvent power by manipulation of temperature, pressure and the presence of a co-solvent modifier. Provided and described are supercritical fluid extraction (SFE) and high-pressure procedures for purifying poloxamers such that the purified polymer is more homogenous with regard to structure (diblock, triblock, etc.), the percentage of molecules without unsaturation, the distribution of molecular weights, and distribution of hydrophobic/hydrophilic (HLB) ratios. The tunability of the processes can be leveraged to effectively remove extraneous components and can be adjusted over time, which can increase the yield of the purified product. The method provided herein uses a solvent system that is variable in its solvation characteristics in order to selectively remove various substances. The methods provide an exemplary way to produce the LCMF poloxamer 188 product, which has the above properties.

Methods herein provide poloxamer preparations that differ from those produced by prior methods. These include the LCMF poloxamer 188 preparation that, upon administration, does not give rise to long circulating material observed with purified poloxamer 188, such as that described in U.S. Pat. No. 5,696,298. The LCMF poloxamer 188 has the molecule size distribution similar to the purified poloxamer 188, but the component molecules produce a preparation that is more hydrophilic than purified poloxamer.

The absence of the long circulating material (LCM) improves the properties of the poloxamer, including faster clearance and other such improved pharmacological properties by virtue of the elimination of the long circulating material. The methods provided herein eliminate unwanted components in a poloxamer preparation, and thereby prepare a more homogenous or uniform poloxamer preparation that exhibits desired therapeutic activity while minimizing or reducing undesired activities. Because commercially available poloxamers have been reported to exhibit toxicity as well as variation in biological activity, a poloxamer preparation that is more uniform and homogenous has reduced toxicity but retains therapeutic efficacy of the main copolymer component.

Provided herein are methods for preparing such poloxamers, and provided are the resulting poloxamers, including the LCMF poloxamer 188. The methods provided herein, in addition to resulting in poloxamer preparations in which low molecular weight (LMW) components are reduced or removed, also result in long circulating material free (LCMF) preparations that are reduced or removed for any component that is or gives rise to a circulating material in the plasma or blood as described herein. Hence, also provided herein are LCMF preparations of poloxamers, and in particular LCMF poloxamer 188. The LCMF poloxamer 188 provided herein can be used for all of the uses known for poloxamer 188.

Provided herein are extraction methods for purifying poloxamers, such as P188, in order to remove or reduce components other than the main component, and thereby decrease the molecular diversity of the preparation. For example, the methods can remove or reduce LMW substances in a poloxamer. It is also found herein, that, in addition to removing or reducing LMW substances, particular methods provided herein also remove or reduce components in a poloxamer preparation that is or gives rise to a long circulating material that has a half-life that is substantially longer than the half-life of the main component in the distribution. The degree of extraction, and components that are extracted, are controlled by the particular temperature, pressure and alkanol concentration employed in the methods as described herein.

The methods provided herein employ a supercritical or subcritical extraction solvent in which the solvent power is controlled by manipulation of temperature, pressure in the presence of a co-solvent modifier. It is found that carbon dioxide is not a particularly efficient extraction solvent of poloxamers, such as P188, but that the presence of a polar co-solvent, such as an alkanol, as a modifier increases the solubilizing efficiency of CO₂ in the extraction solvent. In particular, the methods provided herein are performed in the presence of a polar co-solvent, such as an alkanol, whose concentration is increased in a gradient fashion (e.g., a step-wise gradient or a continuously escalating gradient) as the extraction process progresses. It is found that by employing an alkanol co-solvent whose concentration is increased in this manner, the removal of impurities can be increased, and to a much greater extent than when carbon dioxide is used alone. For example, an extraction method that uses carbon dioxide alone is not capable of removing the unwanted components, such as the LMW components and HMW components as described herein, to the same degree as that achieved by the provided method.

In the methods provides herein for purifying a poloxamer using supercritical fluid extraction, the LMW components or impurities of a poloxamer distribution can be selectively removed with a lower alkanol concentrations (e.g., methanol) and higher pressure than other HMW components in the distribution. As described further below, by increasing the solubilizing power of the extraction solvent, for example by carefully controlling the pressure and concentration of polar solvent, such as an alkanol (e.g., methanol), it also is possible to remove other impurities. In particular, a method is provided employing a gradient of higher concentrations of an alkanol (such as methanol), alone or in conjunction with a decrease in the pressure, that results in the removal of components (e.g. HMW components) in a poloxamer distribution such that, when the resulting product is administered to a subject, it does not result in a long circulating material in the plasma that is observed with the previous P188 products.

There is, however, a tradeoff with respect to the yield of poloxamer. Generally, as the concentration of the alkanol (e.g. methanol) co-solvent increases, the solvating power of the extraction solvent is increased so that more compounds are solubilized and the degree of extraction increases. By increasing the concentration of extraction solvent in a gradient fashion, the reduction of poloxamer yield is minimized, while the purity of the final product is maximized. Typically, the methods provided herein achieve a yield such that the amount of the extracted or purified polymer obtained by the method is at least 55%, 60%, 70%, 75%, 80%, 85%, 90% or more of the starting amount of the poloxamer prior to performance of the method. The resulting poloxamers, however, exhibit a substantially greater purity with a higher percentage of main component in the distribution than the starting material, and without impurities that exhibit toxic side effects or that can result in a long circulating material in the plasma when administered.

The methods can be performed on any poloxamer in which it is desired to increase the purity, for example by decreasing or reducing components that are undesired in the distribution of a polymer. It is within the level of a skilled artisan to choose a particular poloxamer for purification in this manner. Undesired components include any that are or give rise to a material that is toxic or that has a biological activity that is counter or opposing to the desired activity. For example, the poloxamer can be one in which it is desired to reduce or remove LMW components in the poloxamer, for example, any LMW components that result in acute renal side effects, such as elevated creatinine, when administered. The poloxamer also can be one that contains any component, such as a HMW component, that, when administered, is or gives rise to a material that has a half-life in the blood that is different (e.g. longer) than the half-life of the main component in the distribution of the polymer. Such components can increase blood viscosity and red blood cell aggregation, and hence are undesired.

Exemplary of poloxamers for use in the methods include, but are not limited to, poloxamer 188, poloxamer 331, poloxamer 407, and poloxamer 180.5. Typically, the poloxamer is one in which the average molecular weight of the main component is within or about 4,700 Da to 12,800 Da, such as generally 7,680 to 9,510 Da, for example generally 8,400-8,800 Da. In particular, the poloxamer is P188.

For example, the extraction methods provided herein can be employed to purify a P188 preparation, where the P188 preparation has the following chemical formula:

HO(CH₂CH₂O)_{a′}—[CH(CH₃)CH₂O]_{b}—(CH₂CH₂O)ₐH

wherein the hydrophobe represented by (C₃H₆O) has a molecular weight of approximately 1,750 Daltons and an average molecular weight of 7,680 to 9,510 Da, such as generally approximately 8,400-8,800 Daltons. The polyoxyethylene-polyoxypropylene-polyoxyethylene weight ratio of P188 is approximately 4:2:4. P188 has a weight percent of oxyethylene of 81.8±1.9%, and an unsaturation level of 0.026±0.008 mEq/g. P188 preparations for use in the methods herein include commercially available preparations. These include, but are not limited to, Pluronic® F68 (BASF, Florham Park, N.J.) and RheothRx® (developed by Glaxo Wellcome Inc.).

In practicing the extraction methods, the methods include: a) providing a poloxamer (e.g. P188) solution into an extractor vessel, where the poloxamer solution is prepared by dissolving the poloxamer in a first solvent to form the solution; b) admixing an extraction solvent containing a supercritical liquid (e.g. supercritical carbon dioxide) or sub-critical fluid (e.g. high pressure carbon dioxide) and a co-modifier solvent with the solution to form an extraction mixture, wherein the concentration of the co-modifier solvent in the extraction solvent is increased over the time of extraction method; and c) removing the extraction solvent from the extractor vessel to thereby remove the impurities (e.g. LMW and/or other components), from the poloxamer. In the method, the step of dissolving the poloxamer solution in the first solvent can occur prior to charging the solution into an extraction vessel or at the time of charging the solution into an extraction vessel. For example, the poloxamer is dissolved in a separate vessel and then the solution is added to the extraction vessel.

The method can be a high pressure or supercritical fluid extraction method. Typically, the method is performed using supercritical fluid extraction (SFE) using a supercritical liquid in the extraction solvent. A supercritical liquid is any liquid that is heated above the critical temperature and compressed to above the critical pressure. For example, carbon dioxide has a critical temperature of 31.1° C. and a critical pressure of 73.8 bars. Thus, extraction conditions for a supercritical carbon dioxide are above the critical temperature of about 31° C. and critical pressure of about 74 bars. In contrast, high pressure extraction can be achieved under sub-critical conditions in which the pressure exceeds the critical pressure, but the temperature does not exceed the critical temperature.

a. Processes for Extraction

i. Supercritical Methods

In certain instances, the supercritical fluid extraction process employed in the methods provided herein is essentially a solvent extraction process using a supercritical fluid as the solvent. With supercritical fluid, multi-component mixtures can be separated by exploiting the differences in component volatilities and the differences in the specific interactions between the component mixture and supercritical fluid solvent (solvent extraction). In the supercritical region of the phase diagram, a compressible fluid such as carbon dioxide exhibits liquid-like density and much increased solvent capacity that is pressure dependent.

The supercritical fluid exhibits a number of highly advantageous characteristics making it a superior solvent. For example, the tunable solvent power of a supercritical fluid changes rapidly around critical conditions within a certain range. The solvent power of the supercritical fluid, and thus the nature of the component that can be selectively removed during extraction, can be fine-tuned by varying the temperature and pressure of the supercritical fluid solvent.

Another beneficial property of various supercritical fluids is the difference in their critical temperatures and pressures. Each supercritical fluid has a range of solvent power. The tunable solvent power range can be selected by choosing an appropriate supercritical fluid.

In addition to unique solubility characteristics, supercritical fluids exhibit certain physicochemical properties making them more useful. For example, supercritical fluids exhibit liquid-like density, and possess gas-like transport properties such as diffusivity and viscosity. These characteristics also change rapidly around the critical region. Supercritical fluids also have zero surface tension. Since most of the useful supercritical fluids have boiling points around or below ambient temperature, the solvent removal step after purification is simple, energy efficient and does not leave any residual solvents.

The use of solid matrices during extraction provides an additional dimension for a fractionation parameter. A suitable solid matrix provides solvent-matrix and solute-matrix interactions in addition to solute-solvent interactions to enhance the fractionation resolution. The desirable transport properties of supercritical fluids make the process easily scalable for manufacturing. Heat transfer and mass transfer characteristics do not significantly change upon process scale up with supercritical fluid extraction processes. Since the extraction process conditions, such as pressure, temperature, and flow rate, can be precisely controlled, the purification process is reproducible in addition to highly tunable.

In such a method, the extraction solvent can contain a supercritical liquid (e.g. supercritical carbon dioxide), as well as another co-modifier solvent, generally an alkanol, that is increased over time in the extraction. As described above, the presence of the co-modifier solvent can improve the solubility of solutes, such as higher molecular weight or more non-polar solutes, and thereby increase their extraction in the method.

For example, the method provided herein can include: a) providing or introducing a poloxamer (e.g. a poloxamer 188) solution into an extractor vessel, wherein the poloxamer solution is prepared by dissolving the poloxamer in a first alkanol to form the solution; b) admixing an extraction solvent containing a second alkanol and a supercritical liquid, under high pressure and high temperature sufficient to create supercritical liquid conditions, with the solution to form an extraction mixture, wherein the concentration of the second alkanol in the extraction solvent is increased over the time of extraction method; and c) removing the extraction solvent from the extractor vessel to thereby remove the impurities (e.g. LMW component or other components) from the poloxamer preparation. The first and second alkanol can be the same or different. In the method, the step of dissolving the poloxamer solution in the first solvent can occur prior to charging the solution into an extraction vessel or at the time of charging the solution into an extraction vessel. For example, the poloxamer is dissolved in a separate vessel and then the solution is added to the extraction vessel.

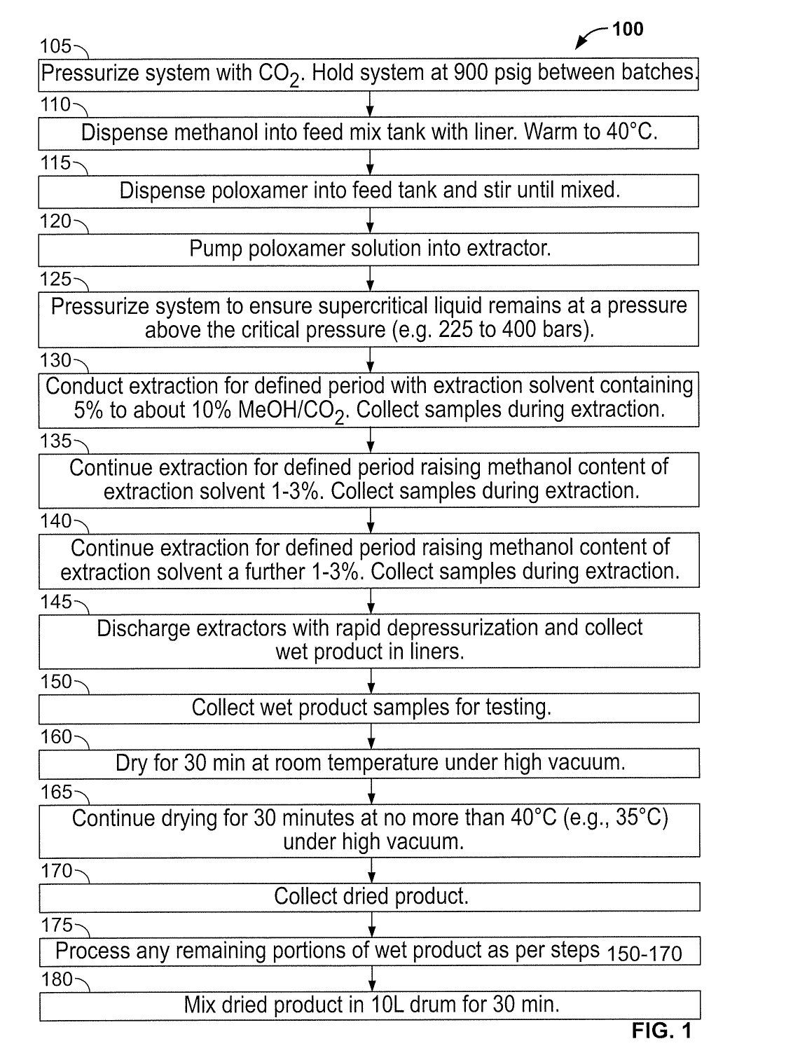

An exemplary process is detailed in FIG. 1. FIG. 1 depicts a process (100) that removes impurities (e.g. LMW component or other components) from a poloxamer preparation. The extraction system is pressurized, as shown in step 105, typically prior to dispensing a first alkanol into the feed mix tank, as shown in step 110. The system is heated to a temperature suitable for the extraction process. The temperature is typically a temperature that is above the critical temperature of the supercritical liquid (e.g. carbon dioxide). Generally, the temperature is approximately 40° C.

Any suitable alkanol or combination of alkanols can be used in the methods provided herein. Examples of suitable alkanols include, but are not limited to, methanol, ethanol, propanol, butanol, and the like. For example, the method provided herein includes an extraction method as described above, wherein the first and the second alkanol are each independently selected from methanol, ethanol, propanol, butanol, pentanol and a combination thereof. In some embodiments, the first alkanol is methanol. In certain instances, methanol is selected as the purification solvent and is the second alkanol in practice of the method. A skilled artisan will appreciate that methanol has relatively low toxicity characteristics. Moreover, methanol has good solubility for poloxamer 188.

The first alkanol (e.g. methanol) is used to form a poloxamer solution according to step 115 in process 100. A poloxamer, such as a P188 preparation, is dispensed into the feed tank and is stirred until mixed with the first alkanol. The amount of poloxamer that is added to the feed tank is a function of the scalability of the extraction method, the size of the extraction vessel, the degree of purity to achieve and other factors within the level of a skilled artisan. For example, non-limiting amounts of poloxamer (e.g. P188) per mL of an extraction vessel can be 0.1 kg to 0.5 kg or 0.2 kg to 0.4 kg. In some examples, in methods of extraction using a 3 L extraction vessel, non-limiting amounts of poloxamer (e.g. P188) can be 0.6 kg to 1.2 kg, such as 0.8 kg to 1.0 kg. In another example, in methods of extraction using a 12 L extraction vessel, non-limiting amounts of poloxamer (e.g. P188) can be 1.5 kg to 5 kg, such as 2 kg to 4 kg. In a further example, in methods of extraction using a 50 L extraction vessel, non-limiting amounts of poloxamer (e.g. P188) can be 8 kg to 20 kg, such as 10 kg to 16 kg or 12 kg to 15 kg. Variations in the amounts are contemplated depending on the particular applications, extraction vessel, purity of the starting material and other considerations within the level of a skilled artisan.

Any suitable ratio of poloxamer and alkanol is contemplated for use in the methods provided herein. The ratio of poloxamer to alkanol, by weight, can be, for example, from about 4:1 to about 1:4, such as from about 3:1 to about 1:3, 2:1 to about 1:2, 1:1 to 4:1 or 1:2 to 1:4. For example, the ratio of poloxamer to alkanol, by weight, can be about 4 to 1, or about 3 to 1, or about 2 to 1, or about 1 to 1, or about 1 to 2, or about 1 to 3 or about 1 to 4. For example, a quantity of poloxamer, such as P188, can be mixed with an equal quantity, by weight, of alkanol (e.g. methanol). A quantity of poloxamer, such as P188, can be mixed with a lesser amount, by weight, of alkanol, such as half the amount, by weight, of alkanol (e.g. methanol). One of skill in the art will appreciate that the appropriate poloxamer to alkanol ratio will depend on poloxamer properties such as solubility in a given alkanol.

After forming a poloxamer/alkanol mixture, all or part of the mixture is pumped into the extractor as shown in step 120. In such examples, the process of preparing the poloxamer solution is performed in a separate vessel from the extractor. A skilled artisan will appreciate that the poloxamer can also be introduced as a solid into the extractor prior to mixing with the first alkanol. Thus, the process of preparing the poloxamer solution can be made directly in the extractor vessel.

The extractor is then pressurized and the extraction solvent is introduced into the extractor as shown in step 125 of process 100. The extraction solvent contains the supercritical liquid. Examples of supercritical liquids include, but are not limited to, carbon dioxide, methane, ethane, propane, ammonia, Freon, water, ethylene, propylene, methanol, ethanol, acetone, and combinations thereof. In some embodiments, the supercritical liquid under pressure is a member selected from carbon dioxide, methane, ethane, propane, ammonia and Freon. In some embodiments, the supercritical liquid under pressure is carbon dioxide (CO₂).

The extraction occurs under high pressure and high temperature to maintain a supercritical liquid condition (e.g. supercritical carbon dioxide). Typically, these are kept constant. At this pressure and temperature, the supercritical liquid (e.g. supercritical carbon dioxide) is provided at a substantially constant flow rate. The flow rate can be varied between 0.5 kg/h to 600 kg/h, such as 1 kg/h to 400 kg/h, 1 kg/h to 250 kg/h, 1 kg/h to 100 kg/h, 1 kg/h to 50 kg/h, 1 kg/h to 20 kg/h, 1 kg/h to 10 kg/h, 10 kg/h to 400 kg/h, 10 kg/h to 250 kg/h, 10 kg/h to 100 kg/h, 10 kg/h to 50 kg/h, 10 kg/h to 20 kg/h, 20 kg/h to 400 kg/h, 20 kg/h to 250 kg/h, 20 kg/h to 100 kg/h, 20 kg/h to 50 kg/h, 50 kg/h to 400 kg/h, 50 kg/h to 250 kg/h, 50 kg/h to 100 kg/h, 100 kg/h to 400 kg/h, 100 kg/h to 200 kg/h or 200 kg/h to 400 kg/h, each inclusive. For example, the flow rate is 20 kg/h to 100 kg/h, inclusive, such as generally about or 100 kg/h.

Any suitable temperature that maintains the supercritical liquid in the supercritical state can be used to conduct the extraction processes. For example, the critical temperature of carbon dioxide is about 31° C. Thus, the extractor vessel is kept at a temperature greater than 31° C. In some embodiments, the extractor vessel has a temperature of 32° C. to 80° C., and generally 32° C. to 60° C. or 32° C. to 60° C., each inclusive. For example, the temperature can be a temperature that is no more than 35° C., 36° C., 37° C., 38° C., 39° C., 40° C., 41° C., 42° C., 43° C., 44° C., 45° C., 50° C. or 60° C. Generally the temperature is greater than 31° C. but no more than 40° C. One of skill in the art will appreciate that the temperature can be varied, depending in part on the composition of the extraction solvent as well as the solubility of a given poloxamer in the solvents employed in the process.

Any suitable pressure can be used in the methods. When supercritical fluid extraction is employed, the system is pressurized at a level to ensure that the supercritical liquid remains at a pressure above the critical pressure. For example, the critical pressure of carbon dioxide is about 74 bars. Thus, the extractor vessel is pressurized to greater than 74 bars. The particular degree of pressure can alter the solubility characteristics of the supercritical liquid. Therefore, the particular pressure chosen can affect the yield and degree of extraction of impurities. Typically, the extractor vessel is pressurized in a range of 125 to 500 bars. In some embodiments, the extractor vessel is pressurized in a range of 200 bars to 400 bars, 200 bars to 340 bars, 200 bars to 300 bars, 200 bars to 280 bars, 200 bars to 260 bars, 200 bars to 240 bars, 200 bars to 220 bars, 220 bars to 400 bars, 220 bars to 340 bars, 220 bars to 300 bars, 220 bars to 280 bars, 220 bars to 260 bars, 220 bars to 240 bars, 240 bars to 400 bars, 240 bars to 340 bars, 240 bars to 300 bars, 240 bars to 280 bars, 240 bars to 260 bars, 260 bars to 400 bars, 260 bars to 340 bars, 260 bars to 300 bars, 260 bars to 280 bars, 280 bars to 400 bars, 280 bars to 340 bars, 280 bars to 300 bars or 300 bars to 340 bars. For example, the extraction vessel can be pressurized at about or at least 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 bars, but generally no more than 500 bars. The extraction vessel can be pressurized, for example, at 310±15 bars.

Typically, in the methods provided herein, the extraction solvent introduced into the extraction vessel also contains an alkanol. Thus, the extraction solvent includes a second alkanol and a supercritical liquid under high pressure and high temperature. The second alkanol acts as a co-solvent modifier of the supercritical liquid to change the solvent characteristics of the supercritical liquid and improve extractability of the solute in the method. Any suitable alkanol or combination of alkanols, as described above, can be used as the second alkanol in the methods provided herein. As described above, in particular examples, the second alkanol is methanol.

Any suitable combination of the second alkanol and the supercritical liquid, such as any described above, can be used in the extraction solvent in the methods. In some embodiments, the extraction solvent includes methanol and carbon dioxide. The second alkanol typically is provided as a percentage (w/w) of the total extraction solvent that is 3% to 20%, and generally 3% to 15%, for example 5% to 12%, 5% to 10%, 5% to 9%, 5% to 8%, 5% to 7%, 7% to 15%, 7% to 12%, 7% to 10%, 7% to 9%, 7% to 8%, 8% to 15%, 8% to 12%, 8% to 10%, 8% to 9%, 9% to 15%, 9% to 12%, 9% to 10%, 10% to 15% or 10% to 12%, each inclusive. The flow rate (kg/h) of the alkanol is a function of the amount of alkanol introduced into the extractor.

For example, a suitable ratio of the alkanol (e.g. methanol) to supercritical liquid (e.g. carbon dioxide) can be selected based on the identity and purity of the poloxamer starting material, or based on other extraction parameters such as temperature or pressure. For example, the ratio of alkanol (e.g. methanol) to supercritical liquid (e.g. carbon dioxide) can be from about 1:100 to about 20:100. In some embodiments, the ratio of alkanol (e.g. methanol) to supercritical liquid (e.g. carbon dioxide) is from about 1:100 to about 15:100. In some embodiments, the ratio of alkanol (e.g. methanol) to supercritical liquid (e.g. carbon dioxide) is from about 2:100 to about 14:100. The ratio of alkanol (e.g., methanol) to supercritical liquid (e.g., carbon dioxide) can be about 3:100, or about 4:100, or about 5:100, or about 6:100, or about 7:100, or about 8:100, or about 9:100, or about 10:100, or about 11:100, or about 12:100, or about 13:100, or about 14:100.

In certain aspects, the extraction can be conducted in an isocratic fashion, wherein the composition of the extraction solvent remains constant throughout the extraction procedure. For example, the amount of supercritical liquid (e.g. carbon dioxide) and alkanol (e.g. methanol) are constant over the time of extraction, for example, by maintaining a constant flow rate of each. Alternatively, the composition of the extraction solvent can be varied over time, typically, by altering (e.g. increasing or decreasing) the amount of the supercritical liquid and/or alkanol components that make up the extraction solvent. Generally, the supercritical liquid (e.g. carbon dioxide) is kept constant while the concentration of the alkanol (e.g. methanol) in the extraction solvent is altered (e.g. increased or decreased) over time of the extraction. The concentrations of the components can be altered by adjusting the flow rate.

In aspects in which the composition of the extraction solvent can be varied over time, a method in which the second alkanol is increased as the extraction process progresses, either as a step-wise gradient or continuously escalating gradient, is beneficial to the method. In certain instances, commercial grade poloxamers have both high molecular weight components and low molecular weight components along with the main product or component. Low alkanol (e.g. methanol) concentrations in high pressure carbon dioxide extraction fluid can selectively remove low molecular weight components. The solubility of impurity enriched extractables, however, is low and it takes time to significantly reduce the low molecular weight components making it less efficient. By increasing the alkanol concentration of the extraction solvent in a gradient fashion (either step-wise gradient or as a continuously escalating gradient), the amount of low molecular weight impurities that are extracted increases.

Also, higher alkanol (e.g. methanol) concentrations increase the solubility, and hence extraction, of higher molecular weight components. Thus, a gradient with successively higher alkanol (e.g. methanol) concentrations in the extraction solvent can progressively extract low molecular weight components, as well as eventually higher molecular weight components, or components that are less soluble. As a non-limiting example to illustrate this, it is believed that a lower alkanol (e.g. methanol) concentration of about 6.6% w/w can remove low molecular weight components. Increasing the concentration of alkanol by 1% to 3% will continue to effect extraction of low molecular weight components, but also result in removal of higher molecular weight components. A further increase in the concentration of alkanol by 1% to 3% will further remove these components as well as other components that have a higher molecular weight and/or were less soluble in the previous extraction solvents.

An extraction solvent with higher alkanol (e.g. methanol) concentrations, however, is not as selective because it provides more solubility for low molecular weight components, but also increases the solubility of other components including the main components. Therefore, the yield of purified product is reduced with high methanol concentrations. By increasing the concentration of the extraction solvent in a gradient fashion, as provided in methods herein, the reduction of poloxamer yield is minimized and the purity of the final product is maximized.

It was found that increasing the methanol concentration step-wise increases the loading capacity of the extractor, thereby increasing the throughput in a given extraction system. A two-phase system forms inside the extractor. A lower phase consists primarily of a mixture of poloxamer and methanol with some dissolved carbon dioxide. The extraction solvent (carbon dioxide with a lower methanol co-solvent fraction) permeates through the lower phase. An upper phase consists primarily of the extraction solvent and the components extracted from the poloxamer. The relative amount of the two phases depends upon methanol concentration in the solvent flow. In a typical extraction system there is adequate head space for proper phase separation of the upper phase. Increasing the methanol co-solvent concentration step-wise during the extraction process leads to higher feed charge into the extractor.

For example, returning to process 100, the composition of the extraction solvent can be varied as shown in steps 130-140. In some embodiments, the percentage of alkanol (e.g. methanol) by weight of the extraction solvent is increased over the course of the method. The methanol content in a methanol/carbon dioxide mixture can be increased in a stepwise fashion or a continuous fashion as the extraction process progresses. In some embodiments, for example, the extraction process for a poloxamer (e.g. P188) starts using about 3% to about 10% by weight (w/w) of an alkanol (e.g. methanol) in an extraction solvent with a supercritical liquid (e.g. carbon dioxide), such as about 5% to about 10%, such as 6% to 8% (e.g., about 6.6% or 7.4%). After a defined period, the alkanol (e.g. methanol) content of the extraction solvent is raised about 1-3%, such as 1-2% (e.g., to 7.6% or 9.1%, respectively). The alkanol (e.g. methanol) content is again subsequently raised about 1-3% such as 1-2% (e.g., to 8.6% or 10.7%, respectively) during a final period.

Any suitable solvent gradient can be used in the methods. For example, the alkanol (e.g. methanol) concentration in supercritical liquid (e.g. carbon dioxide) can be increased from about 5% to about 20% over the course of extraction procedure. The alkanol (e.g. methanol) concentration in supercritical liquid (e.g. carbon dioxide) can be increased from about 5% to about 20%, or from about 5% to about 15%, or from about 5% to about 10%. The alkanol (e.g. methanol) concentration in supercritical liquid (e.g. carbon dioxide) can be increased from about 6% to about 18%, or from about 6% to about 12%, or from about 6% to about 10%. The alkanol (e.g. methanol) concentration in supercritical liquid (e.g. carbon dioxide) can be increased from about 7% to about 18%, or from about 7% to about 12%, or from about 7% to about 10%. The alkanol (e.g. methanol) concentration can be increased in any suitable number of steps. For example, the alkanol (e.g. methanol) concentration can be increased over two steps, or three steps, or four steps, or five steps over the course of the extraction procedure. A skilled artisan will appreciate that other solvent ratios and solvent gradients can be used in the extraction processes.

Time of extraction of the process provided herein can be for any defined period that results in a suitable extraction of material in the preparation while minimizing reductions in poloxamer yield and maximizing purity. The time is a function of the choice of pressure, temperature, second alkanol concentration, and process of providing the extraction solvent (e.g. isocratic or as a gradient of increasing alkanol concentration as described herein). Generally, the extraction proceeds for 5 hours to 50 hours, and generally 10 hours to 30 hours, or 15 hours to 25 hours, each inclusive such as or about 15 hours or 24 hours. The higher the alkanol (e.g. methanol) concentration employed in the method, typically the shorter the time of the extraction. It also is understood that in examples in which a gradient of alkanol is employed in the method, the total time of extraction is divided as a function of the number of gradient steps in the procedure. The extraction in each gradient step can be for the same amount of time or for different times. It is within the level of a skilled artisan to empirically determine the times of extraction to be employed. Samples can be collected during the extraction process to monitor the removal of substances or to determine if adjustment of extraction parameters, such as temperature or the composition of the extraction solvent, is necessary.

In particular, the methods can be used to purify P188. The process can be applied to other polymers as well. For example, in some embodiments, the methods provided herein provide a method for preparing a purified polyoxypropylene/polyoxyethylene composition. The method includes:

a) providing or introducing a polyoxypropylene/polyoxyethylene block copolymer solution into an extractor vessel that is dissolved in a first solvent to form the copolymer solution, wherein the first solvent is methanol, ethanol, propanol, butanol, pentanol or a combination thereof, and the composition comprises:
i) a polyoxypropylene/polyoxyethylene block copolymer having the formula HO(CH₂CH₂O)_{a′}—[CH(CH₃)CH₂O]_{b}—(CH₂CH₂O)ₐH, the mean or average molecular weight of the copolymer is from about 4,000 to about 10,000 Da; and
ii) a plurality of low molecular weight substances having a molecular weight of less than 4,500 Da, wherein the plurality of low molecular weight substances constitutes more that 4% of the total weight of the composition;

b) adding a second solvent to form an extraction mixture, wherein the second solvent contains a supercritical liquid under high pressure and high temperature and an alkanol that is methanol, ethanol, propanol, butanol, pentanol or a combination thereof, and the concentration of the second solvent in the extraction solvent is increased over the time of extraction method; and

c) allowing the extraction mixture to separate to form a plurality of phases comprising a raffinate phase and an extract phase, wherein the raffinate phase and extract phase are separately removed or isolated.

In some cases of the above method, the mean or average molecular weight of the copolymer is from about 7,680 to 9,510 Da, such as generally 8,400-8,800 Da, for example about or at 8,400 Da. In the method, the copolymer solution can be formed in the extractor vessel by the addition of the copolymer and by adding a first solvent to form a solution or a suspension of the copolymer, wherein the first solvent comprises an alkanol selected from the group consisting of methanol, ethanol, propanol, butanol, pentanol and a combination thereof. Alternatively, the addition of the first solvent to the copolymer to form a copolymer solution can be in a separate vessel and the copolymer solution, which is dissolved in the first solvent, is provided or introduced (i.e. charged) into the extractor vessel. In some cases, prior to step c) the method includes stirring the extraction mixture under high pressure and high temperature to extract impurities (e.g. low molecular weight extractable components and other components) from the copolymer composition.

a. High Pressure Methods

The method provided herein to purify a poloxamer (e.g. P188) can be a high pressure fluid extraction method with mixed solvent systems. One of the solvents in the mixed system is a gaseous solvent that can be compressed to liquid at moderate pressures, such as carbon dioxide. For example, the solvent power of methanol or ethanol can be modified with high pressure carbon dioxide (although not necessarily supercritical carbon dioxide i.e., sub-critical) to give the precise solvating power required to selectively remove different fractions of poloxamers.

In such a method, the extraction solvent contains carbon dioxide that is provided under sub-critical conditions, as well as another solvent that is increased over time in the extraction. Accordingly, some embodiments of methods provided herein provide an extraction method for removing impurities in a poloxamer preparation (e.g. low molecular weight components), wherein the method includes:

a) providing or introducing a poloxamer into an extractor vessel that is dissolved in a first solvent to form a solution, wherein the first solvent is selected from among alcohols, aliphatic ketones, aromatic ketones, amines, and mixtures thereof;

b) admixing an extraction solvent with the solution to form an extraction mixture, wherein the extraction solvent comprises high-pressure carbon dioxide and the solvent, and the concentration of the solvent in the extraction solvent is increased over the time of extraction method; and

c) removing the extraction solvent from the extractor vessel to thereby remove the low molecular weight impurities from the poloxamer.

The first and second solvent can be the same or different. In the method, the step of dissolving the poloxamer solution in the first solvent can occur prior to providing or introducing the solution into an extraction vessel or at the time of providing or introducing the solution into an extraction vessel. For example, the poloxamer is dissolved in a separate vessel and then the solution is added to the extraction vessel.

In aspects of the method, the extraction solvent is under sub-critical conditions. In this process, one of the solvents is a gas at room temperature (or close to room temperature) that can be compressed to a liquid at high pressures. Suitable gases that can be compressed to liquids are carbon dioxide, methane, ethane, propane, ammonia, and freon. A typical solvent pair is chosen in such a way that one is a solvent for the component to be removed by extraction, while the other liquid is a non-solvent, or vice-versa. The solvating capacity of the solvent pair is primarily controlled by the ratio of the solvents in the mixture. By passing the solvent pair through the product containing the substances, the relatively more soluble component can be extracted. Gaseous solvents can be pressurized at any suitable sub-critical pressure. For examples, carbon dioxide can be employed at a pressure of from about 25 bars to about 100 bars. The pressure can be about 25, 30, 35, 40, 45, 50, 55, 60, 65, 70, 75, 80, 85, 90, 95, or 100 bars. In some embodiments, the pressure is from about 60 to about 85 bars. In some embodiments, the pressure is about 75 bars.

Any suitable temperature can be used to conduct the extraction processes. In some embodiments, the extractor vessel has a temperature of 10° C. to 80° C. The temperature can be, for example, about 10° C., or about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C. In some embodiments, the extractor vessel has a temperature of from about 20° C. to about 50° C. When purifying poloxamer 188, for example, the extractor vessel can have a temperature of from about 20° C. to about 60° C. (e.g., about 40° C.). Other temperatures can be suitable for purification of poloxamer 188 depending on the extraction apparatus and the chosen extraction parameters. One of skill in the art will appreciate that the temperature can be varied, depending in part on the composition of the extraction solvent as well as the solubility of a given poloxamer in the solvents employed in the process.

Similar to supercritical fluid extraction methods discussed above, the extraction can be conducted in an isocratic fashion, wherein the composition of the extraction solvent remains constant throughout the extraction procedure. For example, the amount of carbon dioxide and solvent (e.g. methanol) in the extraction solvent are constant over the time of extraction, for example, by maintaining a constant flow rate of each. Alternatively, the composition of the extraction solvent can be varied over time, typically by altering (e.g. increasing or decreasing) the amount of the carbon dioxide and/or other solvent (e.g. methanol) that make up the extraction solvent. Generally, the carbon dioxide is kept constant while the concentration of the other solvent (e.g. methanol) in the extraction solvent is altered (e.g. increased or decreased) over time of the extraction. The concentrations of the components can be altered by adjusting the flow rate. The particular concentration of solvent, and the gradient of concentrations employed, can be similar to those discussed above with respect to the supercritical extraction methods. It is within the level of a skilled artisan to adjust concentrations and extraction time appropriately to achieve a desired purity or yield.

Samples can be collected during the extraction process to monitor the removal of substances or to determine if adjustment of extraction parameters, such as temperature or the composition of the extraction solvent, is necessary.

In particular, the methods can be used to purify P188. The process can be applied to other polymers as well. The benefits of the mixed solvent system include effective removal of high molecular weight (HMW) substances and/or low molecular weight (LMW) substances using the mixed system.

In certain embodiments, the provided methods provide a method for preparing a purified polyoxypropylene/composition. The method includes:

a) providing or introducing a polyoxypropylene/polyoxyethylene block copolymer composition into an extractor vessel that is dissolved in a first solvent to form the copolymer solution, wherein the first solvent is an alcohol, aliphatic ketone, aromatic ketone, amines and mixtures thereof, and the composition contains:
i) a polyoxypropylene/polyoxyethylene block copolymer wherein the mean or average molecular weight of the copolymer is from about 4,000 to about 10,000 Da; and
ii) a plurality of low molecular weight substances having a molecular weight of less than 4,000 Da, wherein the plurality of low molecular weight substances constitutes more that 4% of the total weight of the composition;

b) adding a second solvent to form an extraction mixture, wherein the second solvent comprises high-pressure carbon dioxide and the first solvent, and the concentration of the first solvent in the extraction solvent is increased over the time of extraction method;

c) allowing the extraction mixture to separate to form a plurality of phases including a raffinate phase and an extract phase, and the raffinate phase and extract phase are separately removed or isolated.

When the poloxamer is a poloxamer 188 that is purified, the mean or average molecular weight of the copolymer is from about 7,680 to 9,510 Da, such as generally 8,400-8,800 Da, for example about or at 8,400 Da. In the method, the copolymer solution can be formed in the extractor vessel by the addition of the copolymer and by adding a first solvent to form a solution or a suspension of the copolymer, wherein the first solvent comprises an alkanol selected from the group consisting of methanol, ethanol, propanol, butanol, pentanol and a combination thereof. Alternatively, the addition of the first solvent to the copolymer to form a copolymer solution can be in a separate vessel and the copolymer solution, which is dissolved in the first solvent, is provided or introduced (i.e. charged) into the extractor vessel. In some cases, prior to step c) the method includes stirring the extraction mixture under high pressure and high temperature to extract impurities (e.g. low molecular weight extractable components and other components) from the copolymer composition.

In certain aspects, this approach does not have the density variation and permeability characteristics of the supercritical fluid extraction process. However, the solvent recycling is easy and energy efficient. In a typical high pressure extraction, the exit stream containing the extracted component is subjected to lower pressure that causes phase separation and separation of the more volatile solvent as a gas. This leaves the other solvent enriched with the extracted component. The extraction process continues until the extractable component is substantially depleted from the mixture. The gaseous solvent is compressed back into liquid and is available for continued extraction. This solvent recycling process is efficient because the compressible solvent is selected to have complete separation from the solvent mixture with minimum change in the pressure.

b. Extraction Vessel and System

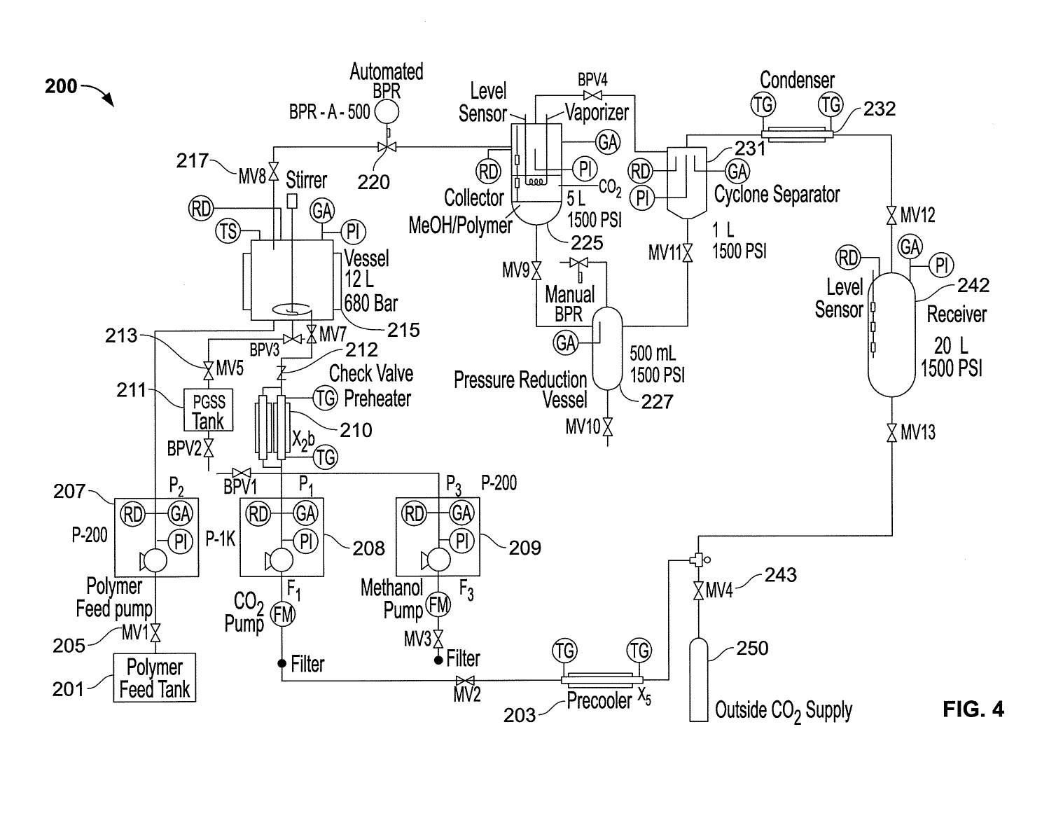

For any of the methods provided herein, system 200 in FIG. 4 represents one embodiment for practice of the provided methods. System 200 is one system that can be used to extract impurities (e.g., LMW substances and/or other components) from the poloxamers using supercritical fluids or sub-supercritical methods. Polymer feed pump 201 is charged with a poloxamer (e.g. P188) to be purified. Poloxamer is transported into polymer feed tank 207 through valve 205. The extractor vessel 215 is used to remove the extracted impurities from the sample, such as LMW substances or other components from the poloxamer. Carbon dioxide (or other supercritical liquid or sub-supercritical liquid) pump 208 is charged with carbon dioxide from outside carbon dioxide supply 250 through valve 243 and pre-cooler 203. Carbon dioxide is pumped from pump 208 into heat exchanger 210 and then into extractor 215. Methanol (or other suitable solvents) is pumped into extractor 215 through pump 209. In such embodiments, methanol and carbon dioxide extract impurities, such as LMW substances or other components, from the poloxamer in extractor 215. After extraction, the purified poloxamer mixture is discharged and collected via rapid depressurization processing. The extracted components are isolated from the solvent stream using collector 225, pressure reduction vessel 227, and cyclone separator 231. Carbon dioxide vapor released during collection in collector 225 can be liquefied and recycled using condenser 232.

In some embodiments, the extraction apparatus can include a solvent distribution system that contains particles of certain shapes forming a “fluidized” bed at the bottom of the extraction vessel. The bed can be supported by a screen or strainer or sintered metal disk. The particles used for the bed can be either perfectly shaped spheres or particles of irregular shape, such as pebbles. Having a smooth surface with less porosity or less surface roughness is preferred for easy cleaning. These advantages can be validated in a pharmaceutical manufacturing processes.

The density of the particles forming the bed is selected to be higher than the solvent density so the bed remains undisturbed by the incoming solvent flow during the extraction process. The size of the particles can be uniform or can have a distribution of different sizes to control the packing density and porosity of the bed. The packing distribution arrangement is designed to provide for balanced, optimum extraction and subsequent coalescence of the solvent particles before exiting the extraction vessel. This facilitates maximum loading of the extractor with poloxamer charge. This can also maximize extraction efficiency, minimize the extraction time, and minimize undesirable carry-over of the purified product out of the extraction vessel.

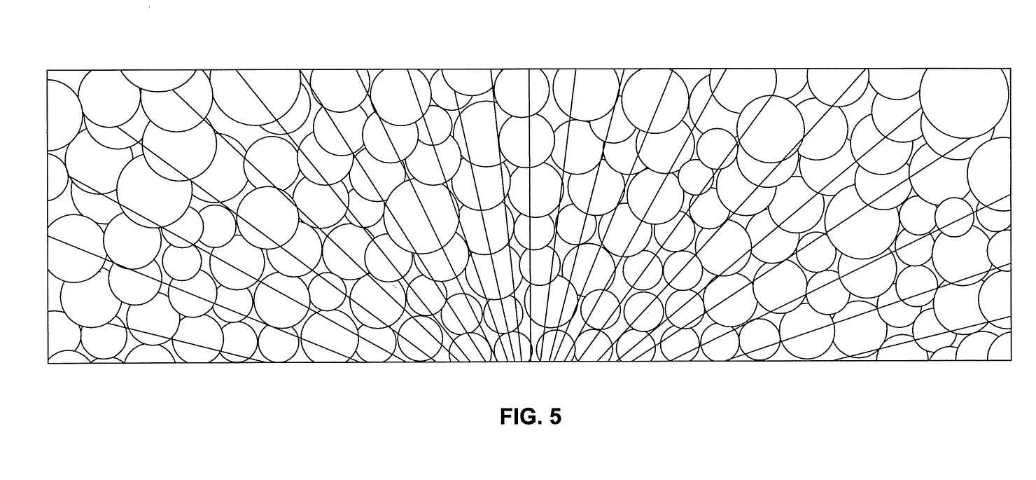

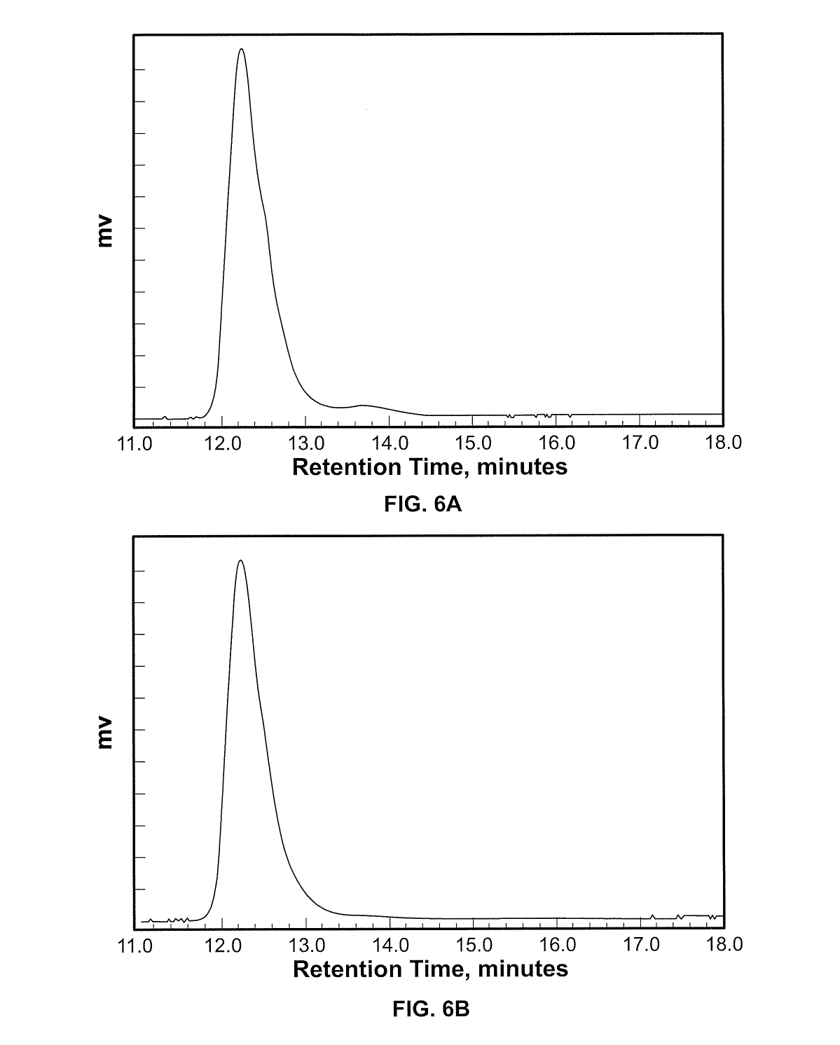

The size of the spheres in the bed is selected based on one or more system properties including the dimensions of the extraction vessel, the residence time of the solvent droplets in the extraction vessel, and the ability of the solvent droplets to coalesce. The diameter of the spheres can range from about 5 mm to about 25 mm. The diameter can be an average diameter, wherein the bed contains spheres of different sizes. Alternatively, all of the spheres in the bed can have the same diameter. An example of the cross section of stainless steel spheres of different sizes in a solvent distribution bed is shown in FIG. 5.

Accordingly, an efficient solvent extraction apparatus is provided. The apparatus includes:

a) a distribution system at the bottom of the extractor, wherein the distribution system comprises a plurality of spheres; and

b) a particle coalescence system at the top of the extractor.

In some embodiments, the plurality of spheres includes metallic spheres, ceramic spheres, or mixtures thereof. In some embodiments, the plurality of spheres are the same size. In some embodiments, the plurality of spheres include spheres of different sizes. In some embodiments, the particle coalescence system includes one or more members selected from a demister pad, a static mister, and a temperature zone.

c. Extraction and Removal of Extractants

Any of the methods provided herein can be performed as a batch method or as a continuous method. In some embodiments, the method is a batch method. A batch method can be performed with extraction vessels of various dimensions and sizes as described above. For example, the equipment train can contain a 120-L high pressure extractor. A Poloxamer (e.g. P188) solution, which is a poloxamer dissolved in an appropriate solvent (e.g. an alkanol solvent, such as methanol), is provided or introduced into the extraction vessel. The extraction solvents, such as any described in the methods above (e.g. supercritical or high-pressure carbon dioxide and methanol) are independently and continuously pumped into the extraction vessel maintained at a controlled temperature, flow, and pressure. Substances are removed by varying the extraction solvent composition as described herein. Alternatively, the extraction process conditions such as temperature and pressure can also be varied independently or in combination. As described below, after substances are removed, the purified product is discharged into a suitably designed cyclone separator to separate the purified product from carbon dioxide gas. The product is dried to remove the residual alkanol solvent.

In some embodiments, the extraction method is a continuous method. In a typical continuous extraction, a poloxamer (e.g. P188) solution, which is a poloxamer dissolved in an appropriate solvent (e.g. an alkanol solvent, such as methanol), is loaded at the midpoint of a high pressure extraction column packed with a suitable packing material. The extraction solvent is pumped through the extraction column from the bottom in counter current fashion. The extracted material, such as LMW substances or other components, are removed at the top of the column while purified product is removed from the bottom of the column. The purified product is continuously collected at the bottom of the extractor column and periodically removed and discharged into a specially designed cyclone separator. The purified polymer particles containing residual methanol are subsequently dried under vacuum.

Depending on the level of purity desired in the purified poloxamer product, the extraction step can be repeated for a given batch. That is, additional portions of the extraction solvent can be introduced into the extractor vessel and removed until a sufficient level of poloxamer purity is obtained. Accordingly, some embodiments of methods provided herein provide extraction methods as described above, wherein after step c, the method further includes repeating steps b and c. Steps b and c can be repeated until the poloxamer is sufficiently pure. For example, steps b and c can be repeated one time, or two times, or three times, or four times, or five times, or in an iterative fashion.

When the poloxamer material is sufficiently pure, the product is prepared for further processing. In some embodiments, the product is handled according to process 100 as summarized in FIG. 1. The product can be discharged from the extractor vessel and collected in an appropriate receiver, as shown in step 145. The wet product can be sampled for testing with respect to purity, chemical stability, or other properties, as shown in step 150. The product can be dried by removing residual solvents under vacuum. Vacuum level can be adjusted to control drying rates. Drying can be conducted at ambient temperature, or at elevated temperatures if necessary. In general, the drying temperature is held below the melting point of the poloxamer. The wet product can be dried in a single lot or in smaller portions as sub-lots. As shown in steps 160-170, drying of the product can be initiated, for example on a sub-lot, under vacuum at ambient temperature. Drying can be then continued at higher temperatures and lower pressures as the process progresses. If necessary, for example if collection was made in sub-lots, any remaining portions of the wet product can be processed in a similar manner, as shown in step 175 of process 100. The resulting product, such as the various sub-lots that have been combined, are mixed in a suitable container, as shown in step 180, and the resulting product can be characterized, stored, transported, or formulated.

Advantageously, the methods disclosed herein effectively recycle carbon dioxide. In particular, supercritical carbon dioxide or high-pressure carbon dioxide can be recovered by subjecting the extract phase to changes in temperature and pressure. In certain embodiments, the methods employed herein have recycling efficiencies of greater than 80%, greater than 90%, and greater than 95%.

In any of such methods, the methods provided herein further include: d) passing the extract phase to a system consisting of several separation vessels; g) isolating the impurities (e.g., low molecular-weight impurities); h) processing the purified material or raffinate and i) recovering the compressed carbon dioxide for reuse.

In any of the methods provided herein, various parameters can be assessed in evaluating the methods and resulting products. For example, parameters such as methanol concentration, gradient profile, temperature, and pressure can be assessed for process optimization. Processes and suitable conditions for drying wet raffinate, such as vacuum level, mixing mode, time, and temperature, also can be assessed.

d. Exemplary Methods

The methods provided herein above result in the generation of particular purified poloxamer preparations, and in particular LCMF P188 preparations. In particular, the methods provided herein can be used to purify a P188 copolymer as described herein that has the formula: HO(CH₂CH₂O)_{a′}—(CH₂CH(CH₃)O)_{b}—(CH₂CH₂O)ₐH, and a mean or average molecular weight of the copolymer that is from 7,680 to 9,510 Da, such as generally 8,400-8,800 Da, for example about or at 8,400 Da, and that contains a plurality of low molecular weight substances having a molecular weight of less than 4,000 Da, wherein the plurality of low molecular weight substances constitutes more that 4% of the total weight of the composition.

In some embodiments, the present methods generate purified poloxamers with less than about 4% low molecular weight components such as less than about 3%, 2% or 1%. Typically, the low molecular weight components include glycols, and volatile degradation impurities such as formaldehyde, acetaldehyde, propionaldehyde, acetone, methanol, and peroxides. In certain instances, the processes herein produce poloxamer substantially free of low molecular weight components, i.e., less than 4%, 3%, 2% or 1% of the foregoing components. The methods also can produce poloxamer substantially free of long circulating material, such that when the purified poloxamer is administered to a subject, there are no components in the poloxamer that are or give rise to a material that has a longer half-life in the blood or plasma more than 5.0-fold the half-life of the main component in the poloxamer distribution, such as generally no more than 4.0-fold, 3.0-fold, 2.0-fold, or 1.5-fold. The following discussion details an exemplary of method that produces such purified poloxamer.

i. Removal of Low Molecular Weight (LMW) Components

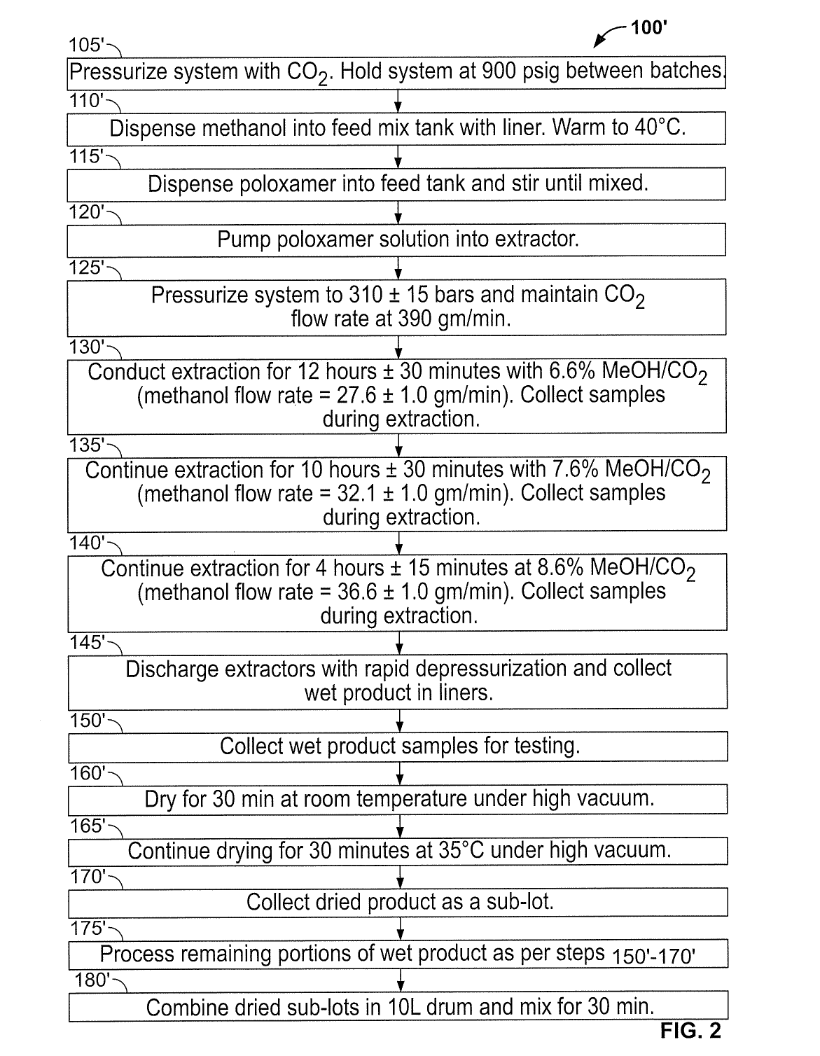

FIG. 2 depicts certain embodiments of the methods herein that provide a process 100′ that is useful for removing LMW substances in a poloxamer. The extraction system is pressurized, as shown in step 105′, prior to dispensing a first alkanol (e.g., methanol) into the feed mix tank, as shown in step 110′. The system is heated to a temperature suitable for the extraction process, which is a temperature above the critical temperature of carbon dioxide used in the process that is about 31° C. Typically, the temperature is no more than 40° C. The temperature is generally kept constant through the process.

The first alkanol (e.g. methanol) is used to form a poloxamer solution according to step 115′ in process 100′. In this process, dispensing of a P188 poloxamer into the feed tank with the alkanol (e.g., methanol,) results in a P188 poloxamer solution that is dissolved in the alkanol (e.g. methanol). The amount of poloxamer for use in the method can be any amount, such as any amount described herein above. After forming a poloxamer/alkanol mixture, all or part of the mixture is pumped into the extractor as shown in step 120′. In some cases, the poloxamer solution can be formed in the extraction vessel by introducing the poloxamer as a solid into the extractor prior to mixing with the alkanol.

The extractor is then pressurized and the extraction solvent is introduced into the extractor as shown in step 125′ of process 100′ The extraction solvent typically contains carbon dioxide and extraction is perform at a temperature greater than the critical temperature of 31° C. as described above and under high pressure greater than the critical pressure of 74 bars. For example, in an exemplary method, the extraction vessel is pressurized to about 310±15 bars, and the carbon dioxide is provided at a flow rate that is 20 kg/h to 50 kg/h, such as generally about or approximately 24 kg/h (i.e. 390 g/min).

The extraction is then conducted in the presence of a second alkanol acting as a co-solvent modifier of the carbon dioxide. The second alkanol, such as methanol, is added in a gradient step-wise fashion such that the concentration of the second alkanol in the extraction solvent is increased over the time of extraction method. For example, the composition of the extraction solvent can be varied as shown in steps 130′-140′. For example, as shown in step 130′, the extraction process for a poloxamer (e.g. P188) starts using about 5% to 7%, by weight (w/w) of an alkanol (e.g. methanol) in an extraction solvent with a supercritical liquid (e.g. carbon dioxide), (e.g., about 6.6%). After a defined period, the alkanol (e.g. methanol) content of the extraction solvent is raised about 1-3%, such as 1% (e.g., to 7.6%). The alkanol (e.g. methanol) content is again subsequently raised about 1-3% such as 1 (e.g., to 8.6%) during a final period. The total time of the extraction method can be 15 hours to 25 hours. Each gradient is run for a portion of the total time.

For a commercially efficient purification process, it desirable to have successively increasing methanol concentrations where the profile is suitably modified to selectively remove most of the low molecular weight components. Residual low molecular weight components can be subsequently removed with high methanol concentrations in a short time. Therefore a stepwise methanol concentration profile where about a 5-10% (e.g., 6.6%) methanol is used for 12 hours, a higher methanol is used for 10 hours and finally an even higher methanol is used for 4 hours to produce purified product in high yields without significantly reducing the overall yield and not enriching the high molecular weight components.

When the poloxamer material is sufficiently pure, the product is prepared for further processing as shown in process 100′. The product can be discharged from the extractor vessel and collected in an appropriate receiver, as shown in step 145′. The wet product can be sampled for testing with respect to purity, chemical stability, or other properties, as shown in step 150′. The product can be dried by removing residual solvents under vacuum as described herein. In an exemplary method as shown in steps 160′-170′, drying can be initiated with a sub-lot under vacuum at ambient temperature and drying can be then continued at higher temperatures and lower pressures as the process progresses. Remaining sub-lots can be processed in a similar manner, as shown in step 175′ of process 100. Sub-lots can be combined and mixed in a suitable container, as shown in step 180′, and the resulting product can be characterized, stored, transported, or formulated.

ii. Preparation of Long Circulating Material Free (LCMF) Poloxamer

FIG. 3 depicts embodiments for preparation of LCMF poloxamer. Certain embodiments of the methods herein provide a process 100″ that generates a poloxamer that does not contain any components that, after administration to a subject, results in a long circulating material in the plasma or blood as described herein. As shown in step 105″, the poloxamer and first alkanol (e.g. methanol) are dispensed into the extractor vessel and to form the poloxamer solution. In this process, dispensing of a P188 poloxamer into the extraction vessel with the alkanol (e.g., methanol,) results in a P188 poloxamer solution that is dissolved in the alkanol (e.g. methanol). The amount of poloxamer for use in the method can be any amount as described herein. In some cases, the poloxamer solution can be formed a separate vessel, and the poloxamer solution transferred to the extractor vessel.

The extraction system is pressurized, as shown in step 110″, after dispensing a first alkanol (e.g. methanol) and poloxamer. As shown in step 115″, the system is heated to a temperature suitable for the extraction process, which is a temperature above the critical temperature of carbon dioxide used in the process, that is about 31° C. Typically, the temperature is between 35° C.-45° C.C. The temperature is generally kept constant through the process. The poloxamer solution is formed under pressurized carbon dioxide of about 49 bars and a temperature of between 35° C. to about or at 45° C. for a defined period, generally less than several hours.

The extractor is then pressurized and the extraction solvent is introduced into the extractor as shown in step 120″ of process 100′. The extraction solvent typically contains carbon dioxide and a second alkanol and extraction is perform at a temperature greater than the critical temperature of 31° C., as described above, and under high pressure, greater than the critical pressure of 74 bars. For example, in an exemplary method, the extraction vessel is pressurized to about 247+15 atm bars (range between 240 to 260 bar), and the carbon dioxide is provided at a flow rate that is 50 kg/h to 120 kg/h, inclusive, such as generally about or approximately 100 kg/h.

The extraction is conducted in the presence of the second alkanol, which acts as a co-solvent modifier of the carbon dioxide. As shown in steps 125″-135″, the second alkanol, such as methanol, is added in a gradient step-wise fashion such that the concentration of the second alkanol in the extraction solvent is increased over the time of extraction method. For example, the composition of the extraction solvent can be varied as shown in steps 125″-135″. For example, as shown in step 125″, the extraction process for a poloxamer (e.g., P188) starts using about 7% to 8% (e.g., about or 7.4%), by weight (w/w) of an alkanol (e.g., methanol) in an extraction solvent with a supercritical liquid (e.g., carbon dioxide). After a defined period, the alkanol (e.g., methanol) content of the extraction solvent is raised about 1-3%, such as up to 2% (e.g., to 9.1%). The alkanol (e.g., methanol) content is again subsequently raised about 1-3% such as up to 2% (e.g., to 10.7%) during a final period. The total time of the extraction method can be 15 hours to 25 hours, inclusive. Each gradient is run for a portion of the total time.

For an extraction process that removes components other than low molecular weight components, including components that, when administered, give rise to longer circulating forms, it desirable to have a process that maximizes the purity and removal of these components while minimizing reductions in yield. It is found that successively increasing alkanol (e.g., methanol) concentrations when starting from a higher concentration of alkanol (e.g., methanol) than in other methods, generally starting at 7% to 8% by weight, the profile is suitably modified to selectively remove these components and low molecular weight components, while minimizing reductions in yield. For example, such an exemplary method can produce yields greater than 55%, and generally greater than 60% or 65%. Residual low molecular weight components can be subsequently removed with high methanol concentrations in a short time. Therefore a stepwise methanol concentration profile where about a 7-8% (e.g., 7.4%) methanol is used for about 3 hours, a higher methanol (e.g., 9.1%) is used for about 4 hours and finally an even higher methanol (e.g., 10.7%) is used for about 8 hours produces a purified product in high yields without significantly reducing the overall yield.

When the poloxamer material is sufficiently pure, the product is prepared for further processing as shown in process 100″. The product can be discharged from the extractor vessel and collected in an appropriate receiver, as shown in step 140″. The product can be precipitated under reduced pressure via particles from gas saturated solutions (PGSS) techniques as shown in step 145″. The product can be dried by removing residual solvents under vacuum as described herein. In an exemplary method, as shown in steps 150″-165″, drying can be initiated under vacuum at high temperatures of between 35° C. to 45° C. ° C. The dried product can be collected as shown in step 160″. The resulting product can be characterized, stored, transported, or formulated as shown in step 165″.

iii. Methods for Confirming the Identity of LCMF Poloxamers

To confirm that a poloxamer 188 preparation made by the methods herein or other methods is an LCMF poloxamer 188, the properties of the poloxamer can be assessed. The properties include, but are not limited to, the absence of a long circulating material upon administration to a human or an animal model, the behavior of the poloxamer in reverse phase (RP)-HPLC compared to a preparation of poloxamer that contains the LCM material such as the poloxamer described in U.S. Pat. No. 5,696,298 and commercially available poloxamer 188 (e.g., those sold under the trademarks Pluronic® F-68, Flocor®, Kolliphor® and Lutrol®), and the behavior in RP-HPLC under the conditions exemplified herein (see i.e., Example 1). Any method that confirms that the preparation lacks LCM material can be used.

### D. PHARMACEUTICAL COMPOSITIONS AND FORMULATIONS

Compositions containing a poloxamer 188 are provided. The poloxamer can be the LCMF preparation described herein and in the copending U.S. provisional applications (see, International PCT application No. (Attorney Docket No. 38645.4003.WO01/4003PC), and U.S. application. Ser. No. (Attorney Docket No. 38645.4003.US02/4003)). The compositions are used in the methods for treating or preventing hemostatic dysfunction, such as for treating bleeding disorders and for the management of drug, disease, trauma and surgically-induced bleeding. In some examples, poloxamer 188 is administered to halt excessive bleeding caused as a result of treatment with an anti-coagulant, such as heparin, or treatment with a thrombolytic agent, such t-PA and/or u-PA. The poloxamer 88 can be administered before such therapy as a prophylactic to reduce potential unwanted bleeding or during, or after, such therapy to modulate the action of thrombolytic agents, or to reduce or eliminate bleeding from the anticoagulant or thrombolytic therapy. In some examples, effective concentrations of P188 are mixed with a suitable pharmaceutical carrier or vehicle for systemic, topical or local administration. The P188 can be mixed with a thrombolytic or anticoagulant agent, or administered simultaneously with, subsequently to or intermittently with a thrombolytic or anticoagulant agent. As described herein, the poloxamers 188 can be used to increase the window of time for initiating thrombolytic therapy for treating stroke and other such events for which thrombolytic therapy is employed.

Pharmaceutical carriers or vehicles suitable for administration of the copolymers include any such carriers known to those skilled in the art to be suitable for the particular mode of administration. Pharmaceutical compositions that include a therapeutically effective amount of P188 also can be provided as a lyophilized powder that is reconstituted, such as with sterile water, immediately prior to administration.

1. Formulations

Pharmaceutical compositions containing P188, such as LCMF P188, can be formulated in any conventional manner by mixing a selected amount of the poloxamer with one or more physiologically acceptable carriers or excipients. Selection of the carrier or excipient is within the skill of the administering professional and can depend upon a number of parameters. These include, for example, the mode of administration (i.e., systemic, oral, nasal, pulmonary, local, topical, or any other mode) and the symptom, disorder, or disease to be treated.

The compound can be suspended in micronized or other suitable form or can be derivatized to produce a more soluble active product. The form of the resulting mixture depends upon a number of factors, including the intended mode of administration and the solubility of P188, such as LCMF P188, in the selected carrier or vehicle. The resulting mixtures are solutions, suspensions, emulsions and other such mixtures, and can be formulated as an non-aqueous or aqueous mixtures, creams, gels, ointments, emulsions, solutions, elixirs, lotions, suspensions, tinctures, pastes, foams, aerosols, irrigations, sprays, suppositories, bandages, or any other formulation suitable for systemic, topical or local administration. For local, internal administration, such as, intramuscular, parenteral or intra-articular administration, the poloxamer can be formulated as a solution suspension in an aqueous-based medium, such as isotonically buffered saline or can be combined with a biocompatible support or bioadhesive intended for internal administration.

Generally, pharmaceutically acceptable compositions are prepared in view of approvals for a regulatory agency or are prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions.

Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. Capsules and cartridges of e.g., gelatin for use in an inhaler or insufflator can be formulated containing a powder mix of a therapeutic compound and a suitable powder base such as lactose or starch. A composition can be formulated as a suppository, with traditional binders and carriers such as triglycerides. Examples of suitable pharmaceutical carriers are described in “Remington's Pharmaceutical Sciences” by E. W. Martin. Such compositions will contain a therapeutically effective amount of P188, in a form described herein, including the LCMF form, together with a suitable amount of carrier so as to provide the form for proper administration to a subject or patient.

The formulation is selected to suit the mode of administration. For example, compositions containing P188, such as LCMF P188, can be formulated for parenteral administration by injection (e.g., by bolus injection or continuous infusion). The injectable compositions can take such forms as suspensions, solutions or emulsions in oily or aqueous vehicles. The sterile injectable preparation also can be a sterile injectable solution or suspension in a non-toxic parenterally-acceptable diluent or solvent. Sterile, fixed oils are conventionally employed as a solvent or suspending medium. For this purpose any bland fixed oil can be employed, including, but not limited to, synthetic mono- or diglycerides, fatty acids (including oleic acid), naturally occurring vegetable oils like sesame oil, coconut oil, peanut oil, cottonseed oil, and other oils, or synthetic fatty vehicles like ethyl oleate. Buffers, preservatives, antioxidants, and the suitable ingredients, can be incorporated as required, or, alternatively, can comprise the formulation.

Formulations suitable for parenteral administration include aqueous and non-aqueous sterile injection solutions, which can contain anti-oxidants, buffers, bacteriostats and solutes that render the formulation compatible with the intended route of administration. The formulations can be presented in unit-dose or multi-dose containers, for example, sealed ampules and vials, prefilled syringes or other delivery devices and can be stored in an aqueous solution, dried or freeze-dried (lyophilized) conditions, requiring only the addition of the sterile liquid carrier, for example, water for injection, immediately prior to use.

P188, such as LCMF P188, can be formulated as the sole pharmaceutically active ingredient in the composition or can be combined with other active ingredients. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811. Liposomal delivery also can include slow release formulations, including pharmaceutical matrices such as collagen gels and liposomes modified with fibronectin (see, for example, Weiner et al. (1985) J Pharm Sci. 74(9): 922-925). The compositions provided herein further can contain one or more adjuvants that facilitate delivery, such as, but not limited to, inert carriers, or colloidal dispersion systems. Representative and non-limiting examples of such inert carriers can be selected from water, isopropyl alcohol, gaseous fluorocarbons, ethyl alcohol, polyvinyl pyrrolidone, propylene glycol, a gel-producing material, stearyl alcohol, stearic acid, spermaceti, sorbitan monooleate, methylcellulose, as well as suitable combinations of two or more thereof.

The P188, such as LCMF P188, is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays provided herein.

2. Dosage

The pharmaceutical compositions containing P188, provided herein, can be formulated for single dosage (direct) administration, multiple dosage administration or for dilution or other modification. The concentrations of the compounds in the formulations are effective for delivery of an amount, upon administration, that is effective for the intended treatment. Those of skill in the art readily can formulate a composition for administration in accord with the methods herein. For example, to formulate a composition, the weight fraction of a compound or mixture thereof is dissolved, suspended, dispersed, or otherwise mixed in a selected vehicle at an effective concentration such that hemostasis is improved. As noted, for the methods herein the compositions should be formulated to deliver, parenterally, a dosage that results in a Cₘₐₓ of more than 1 mg/ml, particularly as more than 5 mg/mL, and up to 10 mg/mL or even higher if necessary. In general, dosages are higher than for those previously described for producing synergistic effects with thrombolytics.

The precise amount or dose of the therapeutic agent administered depends on the route of administration, and other considerations, such as the severity of the bleeding to be stopped or slowed and the weight and general state of the subject, and the subject. Local administration of the therapeutic agent will typically require a smaller dosage than any mode of systemic administration, although the local concentration of the therapeutic agent can, in some cases, be higher following local administration than can be achieved with safety upon systemic administration.

If necessary, a particular dosage and duration and treatment protocol can be empirically determined or extrapolated. For example, exemplary doses of P188, if necessary, can be used as a starting point to determine appropriate dosages for a particular subject and condition. The duration of treatment and the interval between injections will vary with the severity of the bleeding to be stopped and the response of the subject to the treatment, and can be adjusted accordingly. Factors such as the level of activity and half-life of the P188 can be taken into account when making dosage determinations. For example, a P188 product that exhibits a longer half-life can be administered at lower doses and/or less frequently than a P188 product that exhibits a shorter half-life. Particular dosages and regimens can be empirically determined by one of skill in the art.

The effective amounts of a poloxamer, such as P188, can be delivered alone or in combination with other pro-hemostatic agents, e.g., heparin antagonists such as protamine sulfate and anti-fibrinolytic agents such as tranexamic acid. The effective amount can result from administration either once or multiple times by various routes of administration. For the methods herein, the dosage generally is higher than dosages for other indications. It is desirable, for example, for systemic administration to achieve a circulating concentration, particularly a Cₘₐₓ, of greater than 1 mg/ml, particularly greater than 2, 3, 4, 5, 6, 7, 8, 9, or 10 mg/ml. Particularly, for antagonizing thrombolytic induced bleeding and/or anti-coagulant therapy, dosages that produce concentrations of at least 2-10, such as 7-10 mg/ml, in circulation Cₘₐₓ are employed. Dosages for other routes of administration can be extrapolated or deduced therefrom to achieve the result of reducing or stopping bleeding or decreasing clotting time or a similar result, to treat the hemostatic dysfunction and thereby improve hemostasis.

In particular, the poloxamer can be formulated at a concentration ranging from about 10.0 mg/mL to about 200.0 mg/mL or 10.0 to 200.0 mg/ml, such as at or at least 10.0, 15.0, 20.0, 25.0, 30.0, 35.0, 40.0, 45.0, 50.0, 55.0, 60.0, 65.0, 70.0, 75.0, 80.0, 85.0, 90.0, 95.0, 100.0, 105.0, 110.0, 115.0, 120.0, 125.0, 130.0, 135.0, 140.0, 145.0, 150.0, 155.0, 160.0, 165.0, 170.0, 175.0, 180.0, 185.0, 190.0, 195.0 or 200.0 mg/mL, for direct administration. In other embodiments, the poloxamer is administered at a concentration ranging from about 10.0 mg/mL to about 100.0 mg/mL, for example, at or at least 10.0, 15.0, 20.0, 25.0, 30.0, 35.0, 40.0, 45.0, 50.0, 55.0, 60.0, 65.0, 70.0, 75.0, 80.0, 85.0, 90.0, 95.0, or 100.0 mg/mL. In yet other embodiments, the poloxamer is administered at a concentration ranging from about 50.0 mg/mL to about 200.0 mg/mL, such as at or at least 50.0, 55.0, 60.0, 65.0, 70.0, 75.0, 80.0, 85.0, 90.0, 95.0, 100.0, 105.0, 110.0, 115.0, 120.0, 125.0, 130.0, 135.0, 140.0, 145.0, 150.0, 155.0, 160.0, 165.0, 170.0, 175.0, 180.0, 185.0, 190.0, 195.0 or 200.0 mg/mL.

In one example, the poloxamer can be formulated as a sterile, non-pyrogenic solution intended for administration with or without dilution. The final dosage form can be a prepared in a 100 mL vial where the 100 mL contains 15 g of purified poloxamer 188 (150 mg/mL), 308 mg sodium chloride USP, 238 mg sodium citrate USP, 36.6 mg citric acid USP and water for injection USP Qs to 100 mL. The pH of the solution is approximately 6.0 and has an osmolarity of about 312 mOsm/L. For other applications at least 500 mL are prepared with a concentration of 10% to 20%, such as about or at 15% weight of poloxamer preparation/volume of the composition. For example, for intravenous administration, the composition is formulated to achieve the target Cₘₐₓ such that the composition is infused for a period of 30 minutes to up to about or 6 hours, such as 30 minutes to 2 hours. The skilled physician or pharmacist or other skilled person, can select appropriate concentrations for the particular subject, condition treated and target circulating concentration. For the methods herein, as noted, the target circulating maximum concentration typically is greater than 1 mg/ml, 5 mg/ml, or 10 mg/ml, such as 7-10 mg/ml. For local or topical administration concentrations are adjusted accordingly.

When administered separately or as a component of the pharmaceutical composition described herein, the poloxamer is administered at a concentration of between about 0.5% to 20% although more dilute or higher concentrations can be used. For example, the poloxamer can be administered in an amount between about 0.5% to about 20% by weight/volume, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10.0%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5% or 20% by weight/volume. In other embodiments, the poloxamer is administered in an amount between about 0.5% to about 10% by weight/volume, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10.0% by weight/volume. In yet other embodiments, the poloxamer is administered in an amount between about 5% to about 15% by weight/volume, such as 5%, 5.5%, 6%, 6.5%, 7%, 7.5%, 8%, 8.5%, 9%, 9.5%, 10.0%, 10.5%, 11%, 11.5%, 12%, 12.5%, 13%, 13.5%, 14%, 14.5%, or 15% by weight/volume.

In some embodiments, the poloxamer is formulated for administration to a patient at a dosage of about 0.002 to 1,000 mg/kg patient body weight, such as 0.1 to 500 mg/kg patient body weight, 10 to 500 mg/kg patient body weight, 50 to 500 mg/kg patient body weight, or 100 to 500 mg/kg patient body weight, such as about 10, 15, 20, 25, 30, 35, 40, 45, 50, 55, 60, 65, 70, 75, 80, 85, 90, 95, 100, 125, 150, 200, 250, 300, 350, 400, 450, 500, or 1000 mg/kg patient body weight. In particular examples the poloxamer is formulated for administration at a dosage of about 400 mg/kg patient body weight.

In other embodiments, the poloxamer is formulated for administration to a patient at a dosage of about 0.1 to 500 mg/kg patient body weight, such as about 1 to 50 mg/kg patient body weight, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 mg/kg patient body weight. In some examples, the poloxamer is formulated for administration to a patient at a dosage of about 10 mg/kg patient body weight.

The effective amount of poloxamer generally results in a targeted concentration of between about 0.05 mg/mL and about 20 mg/mL in the subject, such as about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL. In some instances, the target concentration of the poloxamer in the subject is about 0.5 mg/ml to about 10 mg/ml, such as about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg/ml. In other instances, the concentration of the poloxamer in the subject is from about 0.2 mg/mL to about 4.0 mg/mL, such as 0.5 mg/mL to about 2.0 mg/mL, or about 0.5, 0.6, 0.7, 0.8, 0.9, 1.0, 1.1, 1.2, 1.3, 1.4, 1.5, 1.6, 1.7, 1.8, 1.9 or 2.0 mg/mL. This range is not intended to be limiting, however, and will vary based on the needs and response of the individual subject as well as the properties of a particular poloxamer chosen for administration. In some embodiments, the concentration of poloxamer promoting hemostasis in the subject is about 0.5 mg/mL.

3. Administration

The effective amounts of a poloxamer can be administered alone or in combination with other pro-hemostatic agents, as therapeutics to reverse a hypo-coagulant condition or as a preventive (i.e. in combination with thrombolytic agents to help prevent bleeding complications). The effective amount can result from administration either once or multiple times by various routes of administration. In general, the amount administered achieves a Cₘₐₓ of greater than 1 mg/ml, particularly greater than 5 mg/ml, or greater than 10 mg/ml in order to achieve a circulation that effects improvements in hemostasis by reducing bleeding or shortening clot formation times or other parameters that relate to improved hemostasis and reduced risk of bleeding. The dosage amount is generally at least 100 mg/kg (weight of the subject), typically at least or at 400 mg/kg or about 400 mg/kg, and can be higher in order to achieve the sufficient circulating concentration to improve hemostasis and/or to prevent or reduce the risk of hemostatic dysfunction, particularly that which can result from administration of thrombolytic and/or anti-coagulation agents, including all described herein. Hence, the compositions and methods provide a way to modulate undesirable side-effects of treatments with agents.

The formulations used in the methods provided herein can be administered by any appropriate route, for example, orally, nasally, pulmonary, parenterally, intravenously, intradermally, subcutaneously, intraarticularly, intracisternally, intraocularly, intraventricularly, intrathecally, intramuscularly, intraperitoneally, intratracheally, by inhalation or topically, as well as by any combination of any two or more thereof, in liquid, semi-liquid or solid form and are formulated in a manner suitable for each route of administration. The poloxamer formulations can be administered once or more than once, such as twice, three times, four times, or any number of times that are required to achieve a therapeutic effect. Multiple administrations can be effected via any route or combination of routes, and can be administered hourly (e.g. every hour or 2 hours, every three hours, every four hours or more), daily, weekly or monthly.

The most suitable route for administration will vary depending upon the disease state to be treated, for example the location of the bleeding or impaired clot formation.

a. Systemic Administration

When administered systemically, the target concentration of the poloxamer in the circulation is generally maintained for about 0.5 hours to about 72 hours, although this time is not meant to be limiting. For example, the administration (infusion) time can be over the course of about 0.5 to 36 hours, 1 to 24 hours, 1 to 12 hours, 1 to 8 hours, or 1 to 4 hours, such as 0.5, 1, 2.5, 3, 3.5, 4, 4.5, 5, 5.5, 6, 6.5, 7, 7.5, 8, 8.5, 9, 9.5, 10, 10.5, 11, 11.5, 12, 16, 20, 24, 30, 36, 48, or 72 hours. The amount of poloxamer dose to achieve the target concentration can be readily determined by one of ordinary skill in the art. Routine procedures that adjust for physiological variables (including, but not limited to, kidney and liver function, age, and body weight and or body surface area) can be used to determine appropriate dosing regimens.

The poloxamer can be administered to the individual (e.g., human or veterinary subject) via intravenous (IV) infusion. For instance, the poloxamer can be administered as a single continuous IV infusion, a plurality of continuous IV infusions, a single IV bolus administration, or a plurality of IV bolus administration. Generally, the poloxamer will be administered by the intravenous route either by bolus or by continuous infusion, although other routes can be used. In some instances, the poloxamer is administered as a local infusion such as a catheter or intra-arterial infusion.

b. Local Administration

In other examples, the route of administration of the poloxamer is selected based on the location of the bleeding. For example, direct local administration can be performed when the patient is experiencing bleeding in a particular region. For example, for treatment of bleeding in the joints, local administration by injection of the therapeutic agent into the joint (i.e., intraarticularly, intravenous or subcutaneous means) can be employed.

In other examples, the poloxamer can be formulated for topical administration for induced cessation of bleeding. For example the poloxamer can be formulated as a cream, gel, or ointment, for topical application, or formulated for administration to the lungs by inhalation or intratracheally, when the bleeding is localized to these areas, such as in a surgical setting. The poloxamer can be formulated with dressings or coated on bandages or other solid or semi-solid devices. The poloxamer can be formulated with other therapeutic agents, particularly agents that improve hemostasis, such as those that seal wounds or close surgical incisions.

In some instances, the poloxamer is administered topically as part of, or in combination with, other pro-hemostatic agents such as microfibrillar collagen or a fibrin sealant. A skilled artisan will understand that the concentration of poloxamer will be adjusted for non-systemic administration, but will be at sufficiently high concentration to effect a reduction in bleeding or to increase clotting. Microfibrillar collagen attracts the patient's natural platelets and uses the normal hemostatic pathway to start the blood clotting process when it comes into contact with the platelets. Fibrin sealants are the effective tissue adhesives and are biocompatible and biodegradable.

In certain instances, the fibrin sealants contain purified, virus-inactivated human fibrinogen, human thrombin, and sometimes added components, such as virus-inactivated human factor XIII and bovine aprotinin. Fibrin sealant, or fibrin “glue,” is an exemplary surgical hemostatic/adhesive material that is used as a sealant. In one embodiment, it is a two-component system in which a solution of concentrated fibrinogen and factor XIII are combined with a solution of thrombin and calcium in order to form a coagulum. Once the thrombin/calcium is combined with the fibrinogen/factor XIII, a fibrin clot forms in seconds.

In one example, a poloxamer formulation is administered topically in combination with a fibrin sealant sold as TISSEEL Fibrin Sealant, which is a 2 component fibrin sealant. The sealer protein solution contains human fibrinogen and a synthetic fibrinolysis inhibitor, aprotinin, which helps prevent premature degradation of fibrin clot. The thrombin solution contains human thrombin and calcium chloride. When mixed together, the 2 components combine and mimic the final stages of the body's natural clotting cascade to form a rubber-like mass that adheres to the wound surface and achieves hemostasis and sealing or gluing of tissues, and the poloxamer assists with the cessation of blood flow once applied.

4. Articles of Manufacture and Kits

Provided are articles of manufacture and method used to promote hemostasis, wherein the article is combined with a poloxamer formulation or composition as described herein. In some examples, the compositions provided herein can be used to make, prepare or to coat a device that is to be applied to a surface of the body of a human or non-human animal, or that is to be placed on a wound in or on the body, or inserted into the body of an human or non-human animal. Hence, provided herein are devices prepared from any of the poloxamer compositions, such as the various P188 compositions provided herein. The device can be an implantable device or other device that is amenable to providing the poloxamer composition, such as P188 and the LCMF P188, to the physiologic environment of a human or non-human animal. Examples of such devices include, but are not limited to, sutures, dressings, bandages, films, meshes, shunts and other implantable devices and dressings. The devices can be prepared by forming the device from or coating the device with a composition provided herein. For example, a thin slab of a liquid composition can be coated onto a bandage, wrap or other dressing. The amount of the poloxmer compositions mixed with or coated on should be an amount that reduces the hemostatic dysfunction, such as to reduce bleeding, to improve hemostasis.

In particular examples, the compositions provided herein can be used to coat virtually any medical device. The coated devices provide a delivery device for local administration of the poloxamer, such as P188, composition. For example, the compositions can be used to coat degradable and non-degradable sutures, orthopedic prostheses such as supporting rod implants, joint prostheses, pins for stabilizing fractures, bone cements and ceramics, tendon reconstruction implants, ligament reconstruction implants, cartilage substitutes, prosthetic implants, cardiovascular implants such as heart valve prostheses, pacemaker components, defibrillator components, angioplasty devices, intravascular stents, acute and in-dwelling catheters, ductus arteriosus closure devices, implants deliverable by cardiac catheters such as atrial and ventricular septal defect closure devices, urologic implants such as urinary catheters and stents, neurosurgical implants such as neurosurgical shunts, ophthalmologic implants such as lens prosthesis, thin ophthalmic sutures, and corneal implants, dental prostheses, tissue scaffolds (particularly soft tissue scaffolds), internal and external wound dressings such as bandages and hernia repair meshes, and other devices and implants known to one of skill in the art. Non-limiting examples of medical devices where the poloxamer composition is coated onto or into or adsorbed to or otherwise combined with the device or dressing composition are described below. In general the weight percentage of the poloxamer can be determined empirically, but it generally is greater than 0.5%, or 1%, by weight of any composition with which it is mixed or otherwise combined, typically higher, such a concentration of 10-20 mg/ml in the composition. As shown here, dosages of the poloxamer must be sufficiently high to effectively promote hemostasis; lower dosages can result in increased blood flow, which can increase bleeding and/or can agonize the effects of thrombolytics. The poloxamer in combination with the dressing, such as a fibrin glue can be provided in a syringe or similar device for direct application.

The articles of manufacture provided herein can contain packaging materials. Packaging materials for use in packaging pharmaceutical products are well known to those of skill in the art. See, for example, U.S. Pat. Nos. 5,323,907 and 5,052,558. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. An array of formulations of the compounds and compositions provided herein are contemplated as are a variety of treatments to employ such devices to effect hemostasis.

a. Sutures

In some examples, the device having a surface coated with a composition provided herein is a suture. Sutures that can be coated include any suture of natural or synthetic origin. The sutures can be absorbable or non-absorbable. Typical suture materials include, by way of example and not limitation, silk, cotton, linen, nylon, PVDF, polyolefin, such as polyethylene and polypropylene, polyesters such as polyethylene terephthalate, homopolymers and copolymers of hydroxycarboxylic acid esters, plain or chromicized collagen, plain or chromicized catgut, polyglycolic acid, polylactic acid, Monocryl and polydioxanone. The sutures can take any convenient form such as braids or twists, and can have a wide range of sizes, such as are commonly employed in the art. The sutures optionally can additionally be coated with one or more antimicrobial substances to reduce the chances of infection, as long as the antimicrobial substances do not interfere with the hemostatic activity of the poloxamer composition.

b. Tissue Adhesives

Tissue adhesives, such as cyanoacrylate adhesives, or “liquid stitches,” are frequently used for wound closure, following injury or surgery, and can be co-formulated with a poloxamer composition described herein to assist with hemostasis and wound healing in the provided methods. Such adhesives include any medical-grade fast-acting glue, including cyanoacrylate polymers, such as, for example, methyl-2-cyanoacrylate, ethyl-2-cyanoacrylate (sold under trademarks, such as, for example, Superglue®, Krazy Glue®), n-butyl cyanoacrylate (sold under trademarks, such as, for example, Indermil® or Histoacryl®), 2-octyl cyanoacrylate (sold under trademarks, such as, for example, LiquiBand®, SurgiSeal®, FloraSeal®, or Dermabond®). See, e.g., published U.S. Application No. 20140056839, which describes an adhesive with poloxamer; for use here and for purposes herein, the amount of poloxamer composition included in the adhesive is significantly greater than 0.5%, particularly, greater than 1%, 2%, 3%, 4%, 5%, 6%, 7%, 8%, 9%, 10%, 15%, 20%, such as at a concentration of 10-20 mg/ml, by weight of the composition, in order to treat or reduce hemostatic dysfunction, and thereby improve hemostasis (i.e., promote clotting, reduce bleeding or otherwise improve the healing/sealing properties of the adhesives).

Liquid adhesives, such as those formulated using polyvinylpyrrolidone, pyroxylin/nitrocellulose, poly(methylacrylate-isobutene-monoisopropylmaleate), and/or acrylate or siloxane polymers, for the topical skin treatment of minor cuts also can be formulated to include a poloxamer composition as described herein.

c. Dressings

Wound dressings, such as bandages or pressure dressings can be coated with a poloxamer composition described herein to promote hemostasis of a wound or surgical site upon which a dressing is applied. Such bandages can be manufactured from any material, such as gauze, cotton, polyester, latex, or latex-free elastic yarns. The bandages can be elastic bandages, which are stretchable and are used to create localized pressure to the wound. Dressings also include adhesive bandages, such as Band-Aids® or Elastoplasts®. Dressings can be manufactured such that the poloxamer composition coats the entire bandage or only a region of the bandage which comes into contact with the wound. It is additionally contemplated that the poloxamer composition can be applied to a dressing immediately prior to application of the dressing to a wound for which hemostasis is desired.

d. Gelatin Sponges

Gelatin sponges can be applied to bleeding areas to quickly stop or reduce bleeding (see, e.g., Kabiri et al., (2011) Current Applied Physics 11(3):457-461). Such sponges can be synthesized to include a poloxamer composition as provided herein or can be coated with a poloxamer composition as provided herein to enhance the hemostatic activity of the sponge.

e. Kits

Poloxamer compositions and formulations also can be provided as kits. Kits can include a pharmaceutical composition described herein and an item for administration. For example, a poloxamer composition or formulation can be supplied with a device for administration, such as a syringe, an inhaler, a dosage cup, a dropper, or an applicator. The kit can, optionally, include instructions for application including dosages, dosing regimens and instructions for modes of administration. Kits also can include a pharmaceutical composition described herein and an item for diagnosis. For example, such kits can include an item for measuring the concentration or amount of poloxamer in a subject.

### E. METHODS OF ASSESSING HEMOSTASIS

Hemostasis can be assessed by any method known to those of skill in the art, these include in vitro and in vivo methods. These assays can be employed to identify subjects for treatment and/or to monitor treatment. Assays for such assessment are known to those of skill in the art and are known to correlate tested activities and effects of therapeutics and in vivo activities (see, e.g., Riley et al., “Laboratory Evaluation of Hemostasis,” Virginia Commonwealth University; published online at pathology.vcu.edu/clinical/coag/Lab %20Hemostasis.pdf). For example, coagulation can be assessed using assays such as the activated partial thromboplastin time (aPTT), Prothrombin time (PT), fibrinogen testing, platelet count, platelet function testing, such as by PFA-100, and thrombodynamics tests. Other assays include, but are not limited to, Ivy skin bleeding time, activated clotting time (ACT), thrombin time (TT) or thrombin clotting time (TCT), bleeding time, mixing test, coagulation factor assays, antiphospholipid antibodies, D-dimer, dilute Russell's viper venom time (dRVVT), thromboelastography (TEG or Sonoclot), and euglobulin lysis time (ELT). In some examples, visual inspection can be used to assess hemostasis. The methods of assessing hemostasis can be performed manually or with instrumentation.

In particular examples, the activated partial thromboplastin time (aPTT), or partial thromboplastin time (PTT), is used to evaluate the status of hemostasis. aPTT measures the efficacy of the intrinsic (contact activation) pathway and the common coagulation pathways. The aPTT test commonly is used to determine heparin dosage. For example, to conduct an aPTT test, blood samples are collected in tubes containing oxalate or citrate to arrest coagulation by sequestering calcium. Phospholipid, an activator, such as silica, celite, kaolin or ellagic acid, and calcium ions are mixed into the plasma sample, and the time is measured until a thrombus (clot) forms. Time to clot formation is typically 30 to 50 seconds in a patient who does not have a bleeding disorder and who has not been administered an anticoagulant agent.

In other examples, the activated clotting time (ACT) test is used to evaluate hemostasis. The ACT test measures the time required for whole blood to clot upon exposure to an activator of the intrinsic pathway, and is commonly used to monitor the effect of heparin administration in immediate need situations. For an ACT test, blood is drawn into a test tube containing activators of coagulation, such as kaolin or celite, in activate coagulation. The ACT test is faster than the activated partial thromboplastin time (aPTT) test, but the results can be affected by platelets.

In other examples, the prothrombin time (PT) is used to evaluate hemostasis. PT measures the efficacy of the extrinsic coagulation pathway. The PT test is commonly used to measure warfarin dosage, liver damage and vitamin K, in addition to determining clotting tendency of blood. PT is typically measured using blood plasma. For example, to conduct a PT test, blood samples are collected in tubes containing oxalate or citrate to arrest coagulation by sequestering calcium. The blood is then fractionated by centrifugation so that the plasma can be isolated. An excess of calcium and tissue factor are added to the plasma fraction and time to formation of a clot is measured optically on an automated instrument. Time to clot formation is typically 10 to 15 seconds in a patient that does not have a bleeding disorder and that has not been administered an anticoagulant agent.

These assays and any others, as well as clinical observation, can be used to identify subjects in need of the treatments provided herein. The status of hemostasis can be assessed, and if the results are indicative of hemostatic dysfunctions, such as abnormal bleeding or a risk thereof, the poloxamer can be administered.

### F. METHODS OF IMPROVING HEMOSTASIS

Provided herein are methods of improving hemostasis in a subject, including in a human or non-human subject, by administering a poloxamer composition at a dosage sufficient to improve hemostasis, such as by reducing or stopping bleeding or increasing clotting or other parameter that reduces hemostatic dysfunction or the risk thereof, to thereby improve hemostasis. In particular, provided are methods for ameliorating hemostatic dysfunction or preventing it by identifying a subject experiencing hemostatic dysfunction or a subject who is undergoing treatment that can result in hemostatic dysfunction or is at risk for hemostatic dysfunction. In particular, involving risk of bleeding; administering to the subject a composition comprising an amount of a polyoxyethylene/polyoxypropylene copolymer having the chemical formula HO(C₂H₄O)_{a′}—(C₃H₆O)_{b}—(C₂H₄O)ₐH to restore, reduce or prevent the hemostatic dysfunction, thereby restoring or improving hemostasis. The amount of copolymer administered achieves a circulating Cₘₐₓ concentration of greater than about 1.0 mg/ml or greater than 1.0 mg/ml, such as at least 5 mg/ml or at least 10 mg/ml; the copolymer preparation has been purified to remove low molecular weight impurities; a′ and a are the same or different and each is an integer, whereby the hydrophile portion represented by (C₂H₄O) constitutes approximately 60% to 90% or 60%-90% by weight of the compound; and b is an integer, whereby the hydrophobe represented by (C₃H₆O) has a molecular weight of about 1,200 Da to about 2,300 Da or 1,200 to 2,300 Da.

1. Subject Selection

Subjects to be selected for the methods provided herein include any human or non-human subject experiencing hemostatic dysfunction, manifested by bleeding, reduced clotting or other such indication or is a risk for thereof. Subjects for the provided methods include subjects receiving thrombolytic therapy or hemostatic therapy or surgical or trauma patients or subjects with clotting disorders. In some examples, the selected subject for methods herein has a bleeding disorder as a result of a trauma, surgery or wound. In such examples, for example, the bleeding is manifested as acute hemarthrosis, chronic hemophilic arthropathy, hematomas, hematuria, central nervous system bleedings, gastrointestinal bleedings, or cerebral hemorrhage, including intracranial hemorrhage, such as subarachnoid hemorrhage. In other examples, the bleeding is due to dental extraction. In other examples herein, where the bleeding disorder is due to surgery, the surgery is heart surgery, angioplasty, lung surgery, abdominal surgery, spinal surgery, brain surgery, vascular surgery, dental surgery, or organ transplant surgery. For example, the surgery is transplant surgery by transplantation of bone marrow, heart, lung, pancreas, or liver.

In other examples, where the subject to be treated contains a defect or impairment in the hemostatic pathway, such as a subject diagnosed with hemophilia, the treatment can include combining the poloxamer with coagulation treatment.

a. Subjects Receiving Thrombolytic, Anticoagulant, or Antithrombotic Therapy

Thrombolytic therapy is administered to subjects at risk for a blood clot or who have a blood clot, such as that which occurs in myocardial infarction, deep vein thrombosis, pulmonary embolism, arterial thrombus, venous thrombus, thromboembolic stroke, and in other indications caused by abnormal blood clotting inside a blood vessel. Thrombolytic therapy is administered to limit damage caused by a blockage or occlusion of a blood vessel. Examples of thrombolytic agents administered for thrombolytic therapy include, but are not limited to, tissue plasminogen activator (t-PA), such as alteplase sold under the trademark Activase®, reteplase (sold under the trademark Retavase®), tenecteplase (sold under the trademark TNKase®), demoteplase, anistreplase (sold under the trademark Eminase®), streptokinase (sold under the trademark Streptase®), anisoylated purified streptokinase activator complex (APSAC), prourokinase, urokinase (sold under the trademark Abbokinase® and Kinlytic®), and direct-acting thrombolytic agents, such as plasmin.

While these agents and treatments therewith are beneficial, there are undesirable side effects. Side effects of thrombolytic therapy can include hemorrhage (e.g., bleeding) at the site of injection or administration or at an alternative location. In some cases internal hemorrhaging can occur, which can lead to larger complications such as a hemorrhagic stroke. Regions that are at high risk of bleeding following administration of a thrombolytic agent include, but are not limited to, a site of a recent surgery, an intracranial site, a gastrointestinal site, a urogenital site and a respiratory tract site.

The methods and compositions provided herein can be used to prevent or reduce or eliminate the side-effects or risk thereof of thrombolytic therapy. Hence, subjects that are administered a thrombolytic agent can be selected for treatment with the methods provided herein. Thus, in some of the provided methods, a patient receiving thrombolytic therapy is treated with the poloxamer, such as P188, and a pharmacological thrombolytic agent. The poloxamer composition can be administered with the thrombolytic agent, after the thrombolytic agent, or even before administration of the thrombolytic agent. The dose can be titrated, such as by monitoring hemostasis, to reduce or control the side effects.

The compositions for effecting this therapy can be packaged in devices to provide ease of delivery, such as, for example, in a dual cylinder syringes, with the P188 in one chamber, and the second agent, such as fibrinogen/thrombin, in the other chamber.

In some examples, the selected subject to be treated by the provided methods is an individual suffering from an acute ischemic stroke (AIS) (e.g., a stroke caused by a blood clot in an artery in the brain), who can be administered a thrombolytic agent such as a tissue plasminogen activator (t-PA) (e.g., alteplase, reteplase and tenecteplase), anistreplase, streptokinase or urokinase, typically within 3 to 4.5 hours of the onset of stroke symptom(s), to break down the blood clot and restore blood flow through the blood vessel. In some examples, the pharmacological thrombolytic therapy can be administered immediately after the stroke, up to 10 hours after the stroke, such as 0 hours, 1 hour, 2 hours, 3 hours, 4 hours, 5 hours, 6 hours, 7 hours, 8 hours, 9 hours or 10 hours after the stroke. In some embodiments, the pharmacological thrombolytic therapy is administered 3.5 hours after the AIS and up to about 10 hours after the AIS, e.g., 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours or 10 hours after the AIS. In other embodiments, the pharmacological thrombolytic therapy is administered 6 hours after the AIS and up to about 10 hours after the AIS, e.g., 6 hours, 6.5 hours, 7 hours, 7.5 hours, 8 hours, 8.5 hours, 9 hours, 9.5 hours or 10 hours after the AIS.

In some embodiments, the selected subject has recently undergone or is undergoing pharmacological anticoagulant therapy with an anticoagulant agent such as, but not limited to, heparin, low molecular weight heparin, warfarin, acenocoumarol, phenprocoumon, atromentin, phenindione, a Factor Xa inhibitor, such as fondaparinux and idraparinux, a direct thrombin inhibitor or other anticoagulant agent. In some examples, the selected patient is a patient that undergoes a surgical procedure. In some examples the patient undergoing the surgical procedure is administered an anticoagulant agent before or during the surgical procedure.

In some examples, the selected patient is administered an anticoagulant agent, such as heparin, for anticoagulation in acute coronary syndrome, atrial fibrillation, deep-vein thrombosis, pulmonary embolism, cardiopulmonary bypass, or hemofiltration. In some examples, the patients are administered high-dose heparin before, during, and/or after procedures that require intense anticoagulant administration, such as, but not limited to, such as cardiac bypass, cardiac angioplasty, thrombolysis, extra-corporeal membrane oxygenation (ECMO) and continuous dialysis.

In some embodiments, the selected subject, for the methods provided herein, has recently undergone or is undergoing pharmacological anti-thrombotic therapy with a cyclooxygenase inhibitor, a thromboxane inhibitor, an ADP re-uptake inhibitor or antagonist, a phosphodiesterase inhibitor, a glycoprotein IIb/IIa antagonist or other anti-platelet agent.

In the provided methods, the poloxamer can be administered prior to, concomitantly with, or after administration of the thrombolytic, anticoagulant, or antithrombotic agent. In some examples, the poloxamer is administered prior to administration of the thrombolytic, anticoagulant, or antithrombotic agent. In other examples, the poloxamer is administered after the thrombolytic, anticoagulant, or antithrombotic agent. In further examples, administration of the thrombolytic, anticoagulant, or antithrombotic agent is stopped prior to administration of the poloxamer and resumed once hemostasis is achieved.

In other embodiments, the poloxamer is administered in a plurality of doses, such as two doses or more. In some instances, the first dose of poloxamer is administered concomitantly with the thrombolytic, anticoagulant, or antithrombotic agent and the second dose is administered after the thrombolytic, anticoagulant, or antithrombotic agent. In other instances, the first dose of the poloxamer is administered after a first dose of the thrombolytic, anticoagulant, or antithrombotic agent and the second dose is administered concomitantly with a second dose of the thrombolytic, anticoagulant, or antithrombotic agent. In yet other instances, the first dose of the poloxamer is administered before the thrombolytic, anticoagulant, or antithrombotic agent and the second dose is administered concomitantly with a second dose of the thrombolytic, anticoagulant, or antithrombotic agent. In some embodiments, the second dose of the poloxamer is administered between about 30 minutes to about 10 hours after the thrombolytic agent. For instance, the poloxamer is given about 30 minutes, 40 minutes, 50 minutes 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours or 10 hours after the administration of the thrombolytic, anticoagulant, or antithrombotic agent.

In the provided methods, the thrombolytic agent can be administered using any method known in the art, such as systemic injection, e.g., by IV injection, or local administration, such as topical application or local injection. The poloxamer, e.g., P188, can be administered using any of the methods described herein.

b. Subjects Receiving Hemostatic Agents

Hemostatic agents are drugs, sealants, glues and adhesives that can promote the cessation of bleeding by, for example, stimulating fibrin formation or inhibiting fibrinolysis. Hemostatic agents can be used as an adjunct to standard surgical techniques to control bleeding. Non-limiting examples of hemostatic agents include aprotinin, nafamostat mesylate, e-aminocaproic acid, tranexamic acid, desmopressin, recombinant activated factor VII, thrombin sealants, fibrin sealants (e.g, Tisseel®, Hemascel® Crosseal®), gelatin-based sealants (e.g, FloSeal®), collagen-based sealants, cellulose-based sealants (e.g., oxidized regenerated cellulose), and glutaraldehyde-based adhesives. Subjects receiving hemostatic agents to control bleeding can be selected for treatment with the methods and compositions provided herein.

In some embodiments, an individual who is receiving a hemostatic agent is administered a poloxamer using the methods described herein to assist with hemostasis. Thus, in some examples, poloxamer, such as P188 or LCMF-P188, can be added to topical hemostatic agents such as fibrin glues, or surgical hemostatic products, used as a coating on sutures, or combined with other procoagulant products to facilitate the formation of a hemostatic clot. In particular embodiments, the hemostatic agent is a fibrin sealant. In the provided methods, poloxamer, such as P188, also can be added to intravenous hemostatic products such as prothrombin complex concentrate or other clotting factor concentrates including factor Xa mutants, to improve hemostasis.

In the provided methods, the poloxamer can be administered prior to, concomitantly with, or after administration of a hemostatic agent. The route of administration of the poloxamer, such as LCMF P188, can be the same or different from the route of administration of the hemostatic agent. In some examples, the poloxamer is administered prior to administration of the hemostatic agent. In other examples, the poloxamer is administered after the hemostatic agent. In yet other embodiments, the poloxamer is administered in two doses. In some instances, the first dose of poloxamer is administered concomitantly with the hemostatic agent and the second dose is administered after the hemostatic agent. In other instances, the first dose of the poloxamer is administered after the hemostatic agent and the second dose is administered concomitantly with a second dose of the hemostatic agent. In yet other instances, the first dose of the poloxamer is administered before the hemostatic agent and the second dose is administered concomitantly with a second dose of the hemostatic agent. In some embodiments, the second dose of the poloxamer is administered between about 30 minutes to about 10 hours after the other agent, e.g., the thrombolytic agent or the hemostatic agent. For instance, the poloxamer is given about 30 minutes, 40 minutes, 50 minutes 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours or 10 hours after the administration of the thrombolytic agent.

In the provided methods, the hemostatic agent can be administered by any method known in the art, such as systemic administration, e.g., by IV injection, or local administration such as topical application or local injection. The poloxamer, such as LCMF P188, can be administered using any of the methods described herein.

2. Combination Therapies

The methods provided herein include administration of poloxamer copolymers to treat or prevent hemostatic dysfunction to improve hemostasis either alone or in combination with other compounds, including but not limited to, fibrinolytic enzymes, anticoagulants, free radical scavengers, anti-inflammatory agents, antibiotics, membrane stabilizers and/or perfusion media. In particular, the poloxamer is administered with another agent such as a thrombolytic agent or a hemostatic agent to modulate or counteract the activities of such agents and thereby limit or prevent the undesirable side effects.

The poloxamer can be administered prior to, concomitantly with, before or after administration of the other agent. In some examples, the poloxamer is administered prior to administration of the other agent. In other examples, the poloxamer is administered after the other agent. In yet other embodiments, the poloxamer is administered in two doses. In some instances, the first dose of poloxamer is administered concomitantly with the other agent and the second dose is administered after the other agent. In other instances, the first dose of the poloxamer is administered after the other agent and the second dose is administered concomitantly with a second dose of the other agent. In yet other instances, the first dose of the poloxamer is administered before the other agent and the second dose is administered concomitantly with a second dose of the other agent. In some embodiments, the second dose of the poloxamer is administered between about 30 minutes to about 10 hours after the other agent, e.g., the thrombolytic agent or the hemostatic agent. For instance, the poloxamer is given about 30 minutes, 40 minutes, 50 minutes 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours or 10 hours after the administration of the other agent. Methods also are provided for treatment of stroke, particularly ischemic stroke, and particularly embolic stroken, by pre-administering any polyoxyethylene/polyoxypropylene copolymers described herein, particularly P188 or LCMF-188 after the stroke and before treatment with, and intermittently with treatment with, a pharmacological thrombolytic agent, such as t-PA and/or u-PA. Administration of the polyoxyethylene/polyoxypropylene copolymer after the stroke, and before, such as 1-5 hours before administration of the pharmacological thrombolytic therapy, improves outcomes, including lesion size and motor function. Dosages include those as set forth above for promoting hemostasis. The following section details general methods for treating stroke with a polyoxyethylene/polyoxypropylene, particularly a P188 preparation, including an LCMF preparation that lack a component that results in a longer half-life product as described herein and in copending U.S. provisional application Ser. No. 62/021,697 and copending U.S. application Ser. No. (attorney docket no. 38645.04003.US03/4003.)

### G. METHODS OF TREATING STROKE

As discussed above, methods for treating stroke are provided. Strokes include hemorrhagic and ischemic strokes, and treatment herein includes administration of a polyoxyethylene/polyoxypropylene copolymer, including any described herein. The polyoxyethylene/polyoxypropylene can be administered alone, or administered with a further agent that is therapeutic for treating strokes.

The dosage of the polyoxyethylene/polyoxypropylene copolymer, such as a P188 poloxamer or a P188 LCMF poloxamer, depends upon the subject and particulars of administration. Dosages include 20 mg/kg to 500 mg/kg patient, such as, but not limited to, 20-50, 20-100, 20-250, 50-100, 50-200, 50-300, 50-1000, 100-200, 100-300, 100-400, 250-450; 300-475, and/or 300-500 mg/kg, and, not limited to, at least 20, 50, 75, 100, 150, 200, 250, 300, 350, 400, 450, 475, 500, 600, 700, 800, 900, and/or 100 mg/kg, and the dosages described in the sections above for improving, inducing or controlling hemostasis. The concentration of copolymer is as described above, and includes, but is not limited to, 5% to 50%, 5% to 25%, such as, for example, 5-10, 5-15, 5-20, 10-15, 10-20%, including, for example, at least 5, 6, 7, 8, 9, 10, 11, 12, 13, 14, 15, 16, 17, 18, 19, 20, 21, 22, 23, 24, 25 and 50%.

Thus, provided are methods for treating a stroke by administering a composition containing a polyoxyethylene/polyoxypropylene copolymer after the stroke. The copolymer is administered alone or with other therapeutic agents for treating the stroke. It is administered before the other agent(s), and can be administered a plurality of times and intermittently or with the other agent(s). The copolymer is administered as soon after the stroke as possible, such as within 5, 4, 3.5, 3, 2, 1 hour(s). Strokes that are treated include acute ischemic stroke (AIS) and hemorrhagic stroke. The additional agents depend upon the type of stroke. The dosage of polyoxyethylene/polyoxypropylene depends upon the type of stroke, and the other agent administered.

Also provided are methods for extending the pharmacologic therapy treatment window for ischemic stroke, by administering a polyoxyethylene/polyoxypropylene copolymer after the stroke; and then administering the pharmacological thrombolytic therapy. The ischemic stroke can be embolytic or thrombolytic. Administering the copolymer after the stroke and before thrombolytic therapy extends the window of time during which the thrombolytic therapy is effective. In addition, it reduces or ameliorates debilitating or adverse consequences of the stroke, compared to administration of the thrombolytic therapy alone.

Administration of the polyoxyethylene/polyoxypropylene copolymer can be repeated with or after administration of the pharmacological thrombolytic therapy. Pharmacological thrombolytic therapy includes treatment with one or more of a tissue plasminogen activator (t-PA), anistreplase, streptokinase, urokinase, or a direct acting thrombolytic. The tissue plasminogen activator (t-PA) can be alteplase, reteplase and/or tenecteplase. Direct acting pharmacological thrombolytic therapy can include administration of plasmin.

The treatment can be effected by administering the polyoxyethylene/polyoxypropylene copolymer once after the stroke, or the therapy can be repeated. The first poloxamer copolymer treatment is administered within 3.5-4 hours after the stroke. Pharmacological thrombolytic therapy, if administered, is administered thereafter, but generally within 5, 4 or 4.5 hours after the stroke. A further treatment or administration of copolymer can follow. Such treatment can be administered at least 6, 7, 8, 9, or 10 hours after the stroke and after the pharmacological thrombolytic therapy.

In particular, provided are methods for treating an acute ischemic stroke by administering a polyoxyethylene/polyoxypropylene copolymer followed by pharmacological thrombolytic therapy. Embodiments are discussed above in the preceding section and in this section. Included are embodiments where the pharmacological thrombolytic therapy is administered immediately after the AIS up to about or 10 hours after the AIS. For example, the pharmacological thrombolytic therapy is administered at least 3.5 hours after the AIS up to about or 10 hours, such as between 3 and 5 hours, inclusive, or 6 and 10 hours, inclusive, after the AIS. Included among the subjects selected for this treatment are those with a high risk of bleeding, such as at a site selected from among a site of recent surgery, an intracranial site, a gastrointestinal site, a urogenital site and a respiratory tract site. Combining the thrombolytic therapy with the copolymer therapy ameliorates the risk and/or reduces the bleeding.

Pharmacological thrombolytic therapy is effected by administration of one or more of tissue plasminogen activator (t-PA), anistreplase, streptokinase, urokinase and a direct acting thrombolytic, such as plasmin. Exemplary thrombolytics include alteplase, reteplase and tenecteplase.

The polyoxyethylene/polyoxypropylene copolymer can be administered at a suitable dosage for effecting treatment. Such dosage depends upon the type of stroke, the other agents administered and the regimen, as well as the particular subject. For example, the copolymer is administered at a dosage to result in the circulation (Cₘₐₓ) of the subject of from about or at 0.05 mg/mL to about or at 10 mg/mL, such as from about 0.2 mg/mL to about 4.0 mg/mL, or at least 0.5 mg/mL.

The polyoxyethylene/polyoxypropylene copolymer includes all those described herein, above, and includes the polyoxyethylene/polyoxypropylene copolymer that has the chemical formula HO(C₂H₄O)_{a′}—(C₃H₆O)_{b}—(C₂H₄O)ₐH, where: a′ and a are the same or different and each is an integer, whereby the hydrophile portion represented by (C₂H₄O) constitutes approximately 60% to 90% or 60%-90% by weight of the compound; and b is an integer, whereby the hydrophobe represented by (C₃H₆O) has a molecular weight of about 1,200 Da to about 2,300 Da or 1,200 to 2,300 Da. In some embodiments the polyoxyethylene/polyoxypropylene copolymer has a polydispersity value that is less than approximately 1.07 or 1.07.

The methods include administering to the subject a polyoxyethylene/polyoxypropylene copolymer having the chemical formula HO(C₂H₄O)_{a′}—(C₃H₆O)_{b}—(C₂H₄O)ₐH in an amount for treating the stroke, where: the copolymer preparation has been purified to remove low molecular weight impurities; a′ and a are the same or different and each is an integer, whereby the hydrophile portion represented by (C₂H₄O) constitutes approximately 60% to 90% or 60%-90% by weight of the compound; b is an integer, whereby the hydrophobe represented by (C₃H₆O) has a molecular weight of about 1,300 to about 2,300 Da or 1,300 to 2,300 Da.

In some embodiments, the polyoxyethylene/polyoxypropylene copolymer has the following chemical formula:

HO(CH₂CH₂O)_{a′}—[CH(CH₃)CH₂O]_{b}—(CH₂CH₂O)ₐH

wherein the hydrophobe (C₃H₆O) has a molecular weight of approximately 1,750 Da and the total molecular weight of the compound is approximately 8,400-8,800 Da. In certain instances, the copolymer has been purified to remove certain low molecular weight impurities so that the polydispersity value is less than approximately 1.07.

In some embodiments, the polyoxyethylene/polyoxypropylene copolymer is a poloxamer with a hydrophobe having a molecular weight of approximately 1,200 to 2,250 Da, such as approximately 1,400 to 2,000 Da, and a hydrophile portion constituting approximately 60% to 100%, or 70% to 90% by weight of the copolymer, poloxamer 188, or variants thereof. In some embodiments the molecular weight of the hydrophobe (C₃H₆O) is about or is 1,750 Da. In some embodiments, the hydrophobe represented by (C₃H₆O) has a molecular weight of 1,500 to 2,100 Da or 1,700 to 1,900 Da. Exemplary copolymers include the poloxamer designated poloxamer 188. In other embodiments, the copolymer is a long-circulating material-free (LCMF) poloxamer, such as a LCMF poloxamer 188, which, when administered to a subject, does not contain any component that is or gives rise in the plasma, of the subject, to a material or component that has a circulating half-life (t_{1/2}) that is more than about 1.5-fold or 1.5-fold greater than the half-life of the main component in the distribution of the copolymer preparation or such that all components have a circulating half-life that is within 5-fold of the half-life of the main component.

In some embodiments, the LCMF poloxamer is a polyoxyethylene/polyoxypropylene copolymer that has the formula: HO(CH₂CH₂O)_{a′}[CH(CH₃)CH₂O]_{b}—(CH₂CH₂O)ₐH, where a or a′ is an integer such that the molecular weight of the hydrophobe (C₃H₆O) is between approximately 1,300 to 2,300 Daltons, wherein a and a′ are the same or different; b is an integer such that the percentage of the hydrophile (C₂H₄O) is between approximately 60% and 90% by weight of the total molecular weight of the copolymer; no more than 1.5% of the total components in the distribution of the copolymer are low molecular weight components having an average molecular weight of less than 4,500 Daltons; no more than 1.5% of the total components in the distribution of the copolymer are high molecular weight components having an average molecular weight of greater than 13,000 Daltons; the polydispersity value of the copolymer is less than approximately or less than 1.07; and the half-life of all components in the distribution of the copolymer, when administered to a subject, is no more than 5.0-fold longer than the half-life of the main component in the distribution of the copolymer.

In some embodiments, the copolymer is an LCMF poloxamer that, when administered to a subject, has a half-life in the plasma of the subject that is no more than 4.0-fold, 3.0-fold, 2.0 fold or 1.5-fold longer than the half-life of the main component in the distribution of the copolymer, such as a poloxamer in which all of the components of the polymeric distribution clear from the circulation at approximately the same rate. For example, where all components in the distribution of the copolymer, when administered to a subject, have a half-life in the plasma of the subject that is no more than the half-life of the main component in the distribution of the copolymer.

In some examples, the half-life in the blood or plasma of all components in the LCMF poloxamer, when administered to a human subject, is such that no component has a half-life that is more than 30 hours, and generally is no more than 25 hours, 20 hours, 15 hours, 10 hours, 9 hours, 8 hours or 7 hours, such as no more than 10 hours. In particular examples, the LCMF poloxamer is a poloxamer 188 in which the percentage of high molecular weight components greater than 13,000 Daltons is no more than or is less than 1%, such as less than 0.9%, less than 0.8%, less than 0.7%, less than 0.6%, less than 0.5% or less of the total distribution of the components, and, when administered, does not result in the distinct component with the longer circulating half-life.

Dosages for each the first and any subsequent administrations of the poloxamer are such that the amount of the copolymer administered achieves a Cₘₐₓ concentration of greater than 0.5 mg/ml, such as at least 1 mg/ml. Exemplary dosages include, but are not limited to, at least 20 mg/kg or at least about 20 mg/kg, at least 50 mg/kg or at least about 50 mg/kg, or is at least 100 mg/kg or at least about 100 mg/kg, or is at least 150 mg/kg or at least about 150 mg/kg. Single dosage ranges can be 100-1000 mg/kg or at least about 100-1000 mg/kg, 50-1200 mg/kg or at least about 50-1200 mg/kg, or at least 800-1000 mg/kg or at least about 800-1000 mg/kg.

The poloxamer can be provided in any suitable composition including any described herewith such as, for example, a 15% weight of poloxamer/volume of the composition is administered and from which an amount to deliver 20-1000 mg/kg per dose to a subject is administered. The copolymer can be a LCMF poloxamer administered to achieve a Cₘₐₓ concentration of greater than 0.5, 1.0 1.5, 2, 3, 4, 5, 6, 7, 8, 9 or 10 mg/ml.

The copolymer composition can be administered parenterally or topically, including, but not limited to, intravenously or subcutaneously. The composition can be administered intravenously over the course of 0.5-3 hours, such as over the course of up to 1 hour. The composition can be administered in volume of about 100 ml to 500 mL, or in a volume of 100 ml to 500 mL or in volume of about 50 ml to 1000 mL, or in a volume of 50 ml to 1000 mL.

### H. EXAMPLES

The following examples are included for illustrative purposes only and are not intended to limit the scope of the invention.

### Example 1

### Preparation and Administration of Long Circulating Material Free (LCMF) Poloxamer 188

A. Supercritical Fluid Extraction (SFE) Process

A multi-step extraction batch process of poloxamer 188 was performed with extraction conducted at a pressure of 247±15 atm (approximately 200-260 bars) and a controlled step-wise increase of methanol of 7.4, 9.1 and 10.7 weight % methanol. Before purification, the poloxamer 188 raw material (BASF Corporation, Washington, New Jersey) was characterized by Gel Permeation Chromatography (GPC). Molecular weight analysis demonstrated that raw material had an average molecular weight of the main peak of about 8,500±750 Da, no more than 6.0% low molecular weight (LMW) species of less than 4,500 Da and no more than 1% high molecular weight species (HMW) greater than 13,000 Da. In addition, the polydispersity was no more than 1.2.

A 50-L, high pressure, stainless steel, extractor vessel was charged with 14 kg of commercial grade poloxamer 188 (BASF Corporation, Washington, New Jersey) and 7 kg of methanol, pressurized with CO₂ (49±10 atm, i.e. 720±147 psi) (Messer France, S.A.S., Lavera, France) and heated to 35° C. to 50° C. for 40-80 minutes until a homogenous solution was obtained. CO₂ (supplied either from a main supply tank or via recycling through an extraction system), was cooled in a heat exchanger and fed into a temperature-controlled, high pressure, stainless steel, solvent reservoir. A high-pressure pump increased the pressure of liquid CO₂ to the desired extraction pressure. The high pressure CO₂ stream was heated to the process temperature by a second heat exchanger. Methanol (Merck KGaA, Darmstadt, Germany) was fed from a main supply tank into the CO₂ solvent stream to produce the extraction methanol/CO₂ cosolvent, which was fed through inlet systems into the extractor vessel as a fine mist at a pressure of 247±15 atm (3600±psi) or 240 to 260 bars and a temperature of 40° C.

A 7.4% methanol/CO₂ extraction cosolvent was percolated through the poloxamer solution for 3 hours at a methanol flow rate typically at 8 kg/hr (range 6.8 kg/hr to 9.2 kg/hr; 108 kg/hr total flow rate). The extraction continued with a 9.1% methanol/CO₂ co-solvent for 4 more hours at a methanol flow rate typically at 10 kg/hour (range of 8.5 kg/hr to 11.5 kg/hr; 110 kg/hr total flow rate). The extraction further continued with a 10.7% methanol/CO₂ cosolvent for 8 more hours at a methanol flow rate typically at 12 kg per hour (range of 10.2 kg/hr to 13.8 kg/hr; 112 kg/hr total flow rate). Throughout the extraction process, extraction of soluble species were continuously extracted from the top of the extractor. The extraction solvent was removed from the top of the extractor and passed through two high pressure, stainless steel, cyclone separators arranged in series to reduce system pressure from 247 atm (3600 psi) to 59 atm (870 psi) and then from 59 atm to 49 atm (720 psi) and to separate CO₂ from the methanolic stream. The separated CO₂ was condensed, passed through the heat exchanger and stored in the solvent reservoir. Pressure of the methanol waste stream was further reduced by passing through another cyclone separator. The purified poloxamer 188 remained in the extractor.

After extraction, the purified poloxamer 188 solution was discharged from the bottom of the extractor into a mixer/dryer unit equipped with a stirrer. The poloxamer 188 product was precipitated under reduced pressure via a Particle from Gas Saturated Solutions (PGSS) technique. The precipitate contained approximately 20% to 35% methanol. The purified poloxamer 188 was dried under vacuum at not more than 40 or 45° C. to remove residual methanol. The feed yield of the product gave an average yield of 65%.

Molecular weight analysis of the purified product as determined by GPC demonstrated that the purified product met the acceptance specifications. There was an average molecular weight of the main peak of about 8,500±750 Da and an average molecular weight average of 8,500±750 Da, no more than 1.5% low molecular weight (LMW) species of less than 4,500 Da and no more than 1.5% high molecular weight species (HMW) greater than 13,000 Da. In addition, the polydispersity was no more than 1.05. Thus, the results showed that the procedures resulted in a measurable reduction in the LMW species, and an improvement in the polydispersity of the purified product.

The resulting purified poloxamer 188 was formulated into a clear, colorless, sterile, non-pyrogenic, aqueous solution containing the purified poloxamer at 150 mg/ml, sodium chloride at 3.08 mg/ml, sodium citrate (dihydrate) at 2.38 mg/ml, and citric acid anhydrous at 0.366 mg/ml in water for injection. The solution was sterile filtered and filled into 100 ml glass vials, covered with a nitrogen blanket, and closed with a butyl rubber stopper and aluminum overseal. The resulting osmolarity of the solution was approximately 312 mOsm/L. The LCMF poloxamer-188 composition did not contain any bacteriostatic agents or preservatives.

B. Characterization of the Plasma Concentration Time Course Following Intravenous Administration of Purified (LCMF) Poloxamer 188 Using HPLC-GPC (Method 1)

Purified LCMF poloxamer 188 generated as described above was administered intravenously to 62 healthy volunteers as part of assessment to determine its effect on the QT/QTc interval. Eight of the 62 subjects were randomly selected for quantitative analysis of the plasma poloxamer levels using an HPLC-GPC method. Following administration, blood samples were obtained by venipuncture into heparin anti-coagulated tubes at baseline, during drug administration (hours 1, 2, 3, 4, 5, and 6) and post administration at hours 1, 1.5, 2, 2.5, 5, 6, and 18. Plasma was separated by centrifugation and stored frozen until analysis. The purified poloxamer 188 was administered as either a high dose of a loading dose of 300 mg/kg/hr for one hour followed by a maintenance dose of 200 mg/kg/hr for 5 hours or a lower dose of 100 mg/kg for 1 hour followed by 30 mg/kg/hr for 5 hours. A mean maximum concentration (Cmax) of the administered purified poloxamer 188 of 0.9 mg/mL was attained by the end of the one hour loading infusion. The mean concentration at steady state (Css) was about 0.4 mg/ml was attained during maintenance infusion. The plasma concentration declined rapidly following discontinuation of the maintenance infusion. The LCMF product purified as described above did not demonstrate the long circulating higher molecular weight material, observed with prior poloxamer 188 and as defined herein, in the plasma.

To confirm the absence of such long circulating material in plasma, plasma from subjects receiving the higher dose were similarly studied using HPLC-GPC (gel permeation chromatography). FIGS. 7A and 7B show serial HPLC-GPC of plasma obtained at various time points following administration of the purified LCMF poloxamer 188 for a single subject. FIG. 7A shows the chromatograms at all time points, while FIG. 7B shows selected time points for comparison. In both figures, the chromatogram is enlarged to show the high molecular weight portion (19.8 K Daltons−12.4 K Daltons) of the polymeric distribution. Also shown are the main peak portion (12.8−4.7 K Da) and the lower molecular weight portion (4.7−2.5 K Da). The HPLC-GPC method quantifies plasma levels based on the height of the eluting peak relative to standards of known concentration (i.e. the higher the eluting peak, the higher the plasma level). The GPC method also identifies the molecular weight range by comparison of the sample elution time to that of standards of known molecular weight.

The chromatograms show that over time the high molecular weight portion of the poloxamer 188 polymeric distribution declines in relative proportion to the main peak and lower molecular weight components. Thus, the polymeric distribution shows that the high molecular weight portion clears from the circulation in a substantially uniform manner. The results also show that the higher molecular weight species do not exhibit a longer circulating half-life (relative to the other polymeric components) and do not accumulate in the circulation following intravenous administration.

C. Comparison of the Plasma Concentration Time Course Following Intravenous Administration of Purified LCMF Poloxamer 188 and Purified LCM-Containing Poloxamer 188 by HPLC-GPC

1. Administration of the Long Circulating Material (LCM)-Containing Poloxamer 188

The (LCM-containing) purified poloxamer 188 was administered to 6 healthy volunteers as an intravenous loading dose of 100 mg/kg/hr for one hour followed by 30 mg/kg/hr for 48 hours as part of a safety and pharmacokinetics study (Grindel et al). Blood samples were obtained by venipuncture into EDTA anticoagulated tubes prior to drug administration (baseline), during administration (at 1 hour, 6 hours, 12 hours 18 hour 24 hours 36 and 48 hours) and at 30 minutes, 1 hour, 1.5 hours, 2 hours, 4 hours, 6 hours, 8 hours, 12 hours, 14 hours, 20 hours and 24 hours post drug administration. Plasma was separated and stored frozen until analysis using an HPLC-GPC method. Analysis of the plasma samples revealed the clearance kinetics of the main peak and the HMW peak for the (LCM-containing) purified poloxamer 188

a. HMW Peak (the Long Circulating Material)

Following administration at the above dose, the HMW component (detected in the HPLC-GPC assay as a peak of approximately 16,000 Daltons) was accumulating during the drug administration period and did not reach its mean Cmax concentration of 225 μg/ml (n=6) until 2 hours after the end of drug administration. By 6 hours after discontinuation of infusion, mean plasma levels remained at 202 ug/ml, a concentration that had declined by only about 10% from the Cmax value. Over the 24 hour post infusion blood collection period, mean plasma levels only declined by 22.5% to a plasma concentration of 165 μg/ml. Based on these changes in the plasma concentration time course an elimination half-life of >48 hours is estimated.

b. Main Peak

Following administration at the dose above, the main peak achieved an apparent mean steady state concentration of 522 μg/ml (n=6) that was maintained during drug infusion. One hour after discontinuation of infusion, plasma levels dropped from the steady state concentration by 52% to 255 μg/ml. By 6 hours after discontinuation, plasma levels had dropped by 85% to 81 μg/ml. By 24 hours post infusion, plasma levels declined by 96% to a plasma concentration of about 19 μg/ml (n=6). Based on these changes in the plasma concentration time course the half-life is estimated to be about 5 hours.

2. LCMF Poloxamer 188 (Prepared as Described Above)

LCMF poloxamer was administered to 62 healthy volunteers at a dose of 300 mg/kg for one hour followed by 200 mg/kg/hr for 5 hours as part of the assessment to determine its effect on the QT/QTc interval as previously described. Eight of the 62 subjects were randomly selected for quantitative analysis of the plasma poloxamer levels using a similar HPLC-GPC method as described in part (B) above but with improved linearity at lower plasma levels.

a. HMW Peak

Following administration at the above dose, the HMW component, which was detected in the HPLC-GPC assay as a peak of approximately 16,000 Daltons, accumulated to a small extent during drug administration, and achieved its Cmax (mean value of 117 μg/ml, n=8) by end infusion. By 1 hour after discontinuation of drug administration, plasma levels had declined by 27% from the Cmax value to 86 μg/ml. By 6 hours after the end of drug administration, mean plasma levels had declined by 71% from the Cmax value to 34 μg/ml. By 18 hours after the end of infusion, the mean plasma level had declined by 82% to a concentration of 19 μg/ml (n=8). Based on these changes in the plasma concentration over time, the elimination half-life for the HMW component was estimated to be between 6-9 hours.

b. Main Peak

Following administration at the dose above, the main peak achieved an apparent mean steady state concentration of 2,637 μg/ml that was maintained during the 6 hour infusion period (n=8). One hour after discontinuation of infusion, mean plasma levels had decreased from steady state by 67% to 872 μg/ml and by 6 hours after discontinuation, mean plasma levels had declined by 93% (from steady state) to 184 μg/ml. By 18 hours after discontinuation of infusion, mean plasma levels declined by over 98% (from steady state) to a plasma concentration of about 34 μg/ml (n=6). Based on these changes in the plasma concentration time course, the elimination half-life is estimated to be about 3 hours.

c. Summary Comparison Table

A comparison of the relative rates of clearance from the plasma at similar time points following administration is shown in TABLE 1 below. The data demonstrate a marked difference in the rate of decline in plasma concentration between (LCM-containing) purified poloxamer 188 and the LCMF poloxamer 188, demonstrating that LCMF poloxamer 188 clears faster. The difference is apparent for the HMW peak and for the main peak. The difference is most apparent for the HMW peak. This shows that the LCMF poloxamer is different from the LCM-containing poloxamer of the prior art.

TABLE 1

| | HMW Peak | | Main Peak | |
|---|---|---|---|---|
| | | LCM- | | (LCM- |
| | | (containing) | | containing) |
| | | purified | | purified |
| | | poloxamer | | poloxamer |
| | LCMF | 188 | LCMF | 188 |
| % decrease | 27 | 0 | 67 | 52 |
| 1 hr | 71 | 10 | 93 | 85 |
| Apparent | 6-9 hours | >48 hours | About 3 hours | About |
| elimination t½ | | | | 5 hours |

D. Analytical Data Confirming that Purified LCMF Poloxamer 188 is Different from Purified Poloxamer 188 Containing LCM

1. Analytical Test (RP-HPLC Assay) to Compare Various Poloxamers

In reversed phase chromatography there is a hydrophobic stationary phase (the column) and a more polar mobile phase. Because of this “reversed” phase condition, RP-HPLC is commonly used to separate compounds based on relative hydrophobicity. More hydrophobic compounds exhibit a longer column retention time compared to more hydrophilic compounds.

The following HPLC conditions were used to compare column retention times for various poloxamers with known differences in their hydrophilic/lipophilic balance (HLB), along with purified poloxamer 188 containing LCM and the LCMF poloxamer 188:

| Column | Xterra RP18, 3.5 um, 4.6 × 100 mm | |
|---|---|---|
| Mobile Phase | A: 0.1% HOAc in Water | |
| | B: Acetonitrile | |
| | Time | % B |
| Gradient | 0 | 50 |
| | 1.0 | 50 |
| | 15.0 | 90 |
| | 16.0 | 90 |
| | 16.1 | 50 |
| | 20.0 | 50 |

| | |
|---|---|
| Flow Rate | 0.50 ml/min |
| Column Temp | 40° C. |
| ELS*Detection | N₂: 0.5 liter/minute, Nebulizer: 75° C., |
| | Evaporator: 75° C. |
| Sample Preparation | Drug Product - No dilution |
| | Purified Poloxamer 188, 150 mg/mL in 10 mM |
| | NaCitrate pH 6 |
| Injection Volume | 10 μL |

*ELS = evaporative light scattering

2. Results

The results show that the LCMF poloxamer 188 is different from the prior art purified poloxamer 188. It has different pharmacokinetic properties, which reflect that it is more hydrophilic than the prior art material that contains the long circulating material.

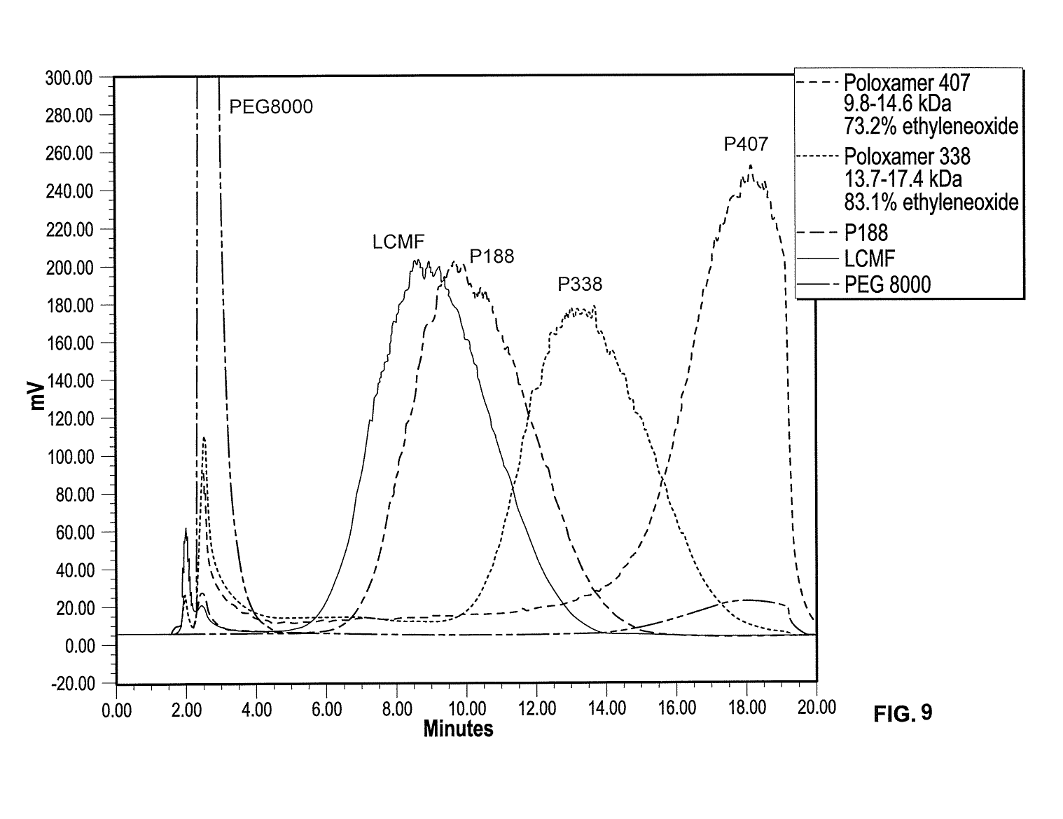

FIG. 9 shows the RP-HPLC chromatograms for a highly hydrophilic polymer (PEG 8000), the LCMF poloxamer 188, the LCM-containing purified poloxamer 188, and two poloxamers with decreasing HLB values (increasing hydrophobicity), Poloxamer 338 and Poloxamer 407, respectively. The most hydrophilic polymer, PEG 8000, exhibits little retention on the column consistent with its highly hydrophilic nature. Poloxamer 338 (HLB>24) and Poloxamer 407 (HLB 18-23) exhibit far longer retention times (add the t_{R} and k′ values) in accord with their known HLB values. The LCMF purified poloxamer 188 elutes more quickly than the LCM-containing purified poloxamer 188, (the average t_{R} and k′ for LCMF purified poloxamer is about 8.8 (8.807) and about 3.2 (3.202), respectively, compared to about 10.0 (9.883) and 3.7 (3.697) for LCM containing purified poloxamer) indicating that the LCMF poloxamer 188 is relatively more hydrophilic than the LCM containing purified poloxamer 188.

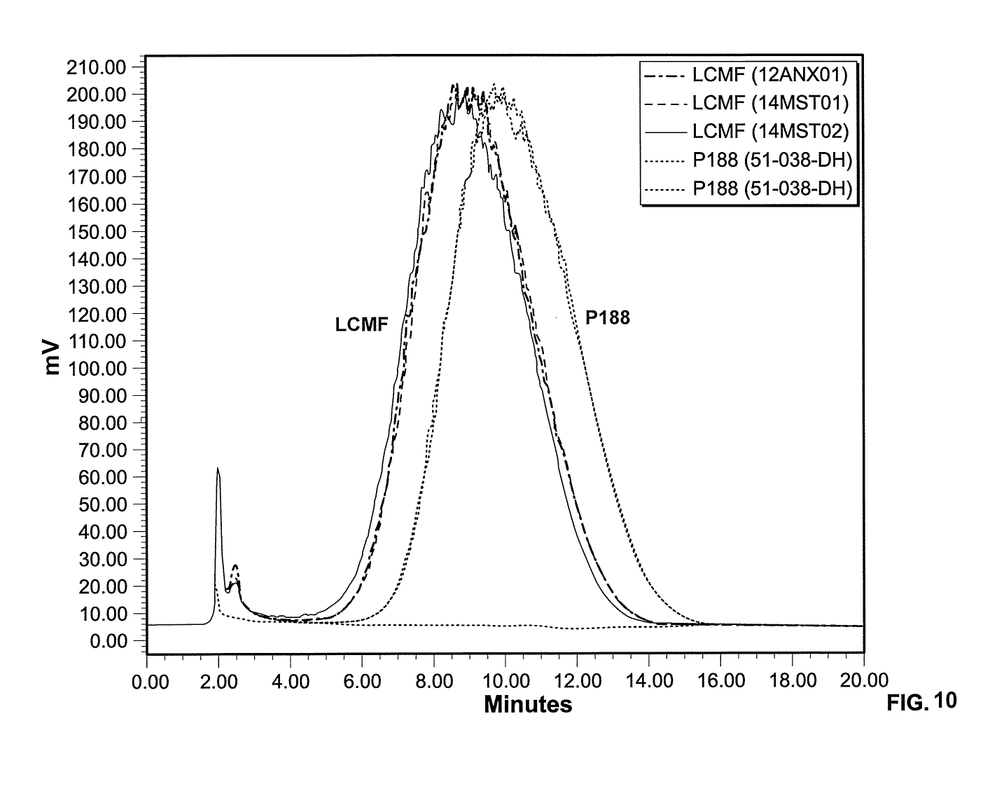

FIG. 10 shows the chromatograms for 3 different lots of purified LCMF poloxamer 188 and two (2) different lots of purified (LCM-containing) poloxamer 188. These results demonstrate a robust reproducibility for the different lots of materials, and show that the difference between the two materials cannot be accounted for by assay variability. These results demonstrate that the polymeric distribution of LCMF poloxamer 188 is more hydrophilic than purified poloxamer 188.

3. The Different Pharmacokinetic Behavior of the LCMF Purified Poloxamer and the LCM-Containing Poloxamer Correlate with the Differences in their Hydrophilicity

As described herein (see, e.g., Example 1B, above, and FIGS. 9-10) and TABLE 1), the LCMF poloxamer 188 exhibits a markedly different pharmacokinetic behavior following administration to human subjects when compared to purified poloxamer 188, which contains the long circulating material (LCM) following in vivo administration. The data provided in this example indicate that LCMF poloxamer 188 is more hydrophilic compared to purified poloxamer 188 that gives rise to the long circulating material.

The polymeric size distribution of purified variants of poloxamer 188 purified LCM-containing poloxamer 188, and the LCMF poloxamer 188) is similar with regard to size as shown by HPLC-GPC. Both meet the criteria:

| | Acceptance | |
|---|---|---|
| Test Attribute | Criteria | Test Method |
| Molecular Weight | | HPLC-GPC |
| Analysis | | |
| Peak MW | 8500 ± 750 Da | |
| Weight Average MW | 8500 ± 750 Da | |
| % LMW (<4500 Da) | NMT* 1.5% | |
| % HMW (>13000 Da) | NMT 1.5% | |
| Polydispersity | NMT 1.05 | |

*NMT = Not More Than

While the polymeric size distribution, as shown by HPLC-GPC, of both purified poloxamers is similar, as demonstrated by the RP-HPLC herein, the molecules that comprise the polymeric distribution of LCMF poloxamer 188 are more hydrophilic. When injected into an animal, a more hydrophilic polymeric distribution clears from the circulation at a faster rate. This accounts for the decreased presence of a long circulating material in the LCMF poloxamer 188 preparation. The results also indicate that, as observed and described above, the main peak of the polymeric distribution clears faster. For example, the plasma concentration time course data from a clinical trial show a shorter elimination half-life for the main peak and the high molecular weight peak of the LCMF poloxamer 188 compared to the purified poloxamer 188 containing LCM.

Since the rheologic, cytoprotective, anti-adhesive and antithrombotic effects of P188 are optimal within the predominant or main copolymers of the distribution, which are approximately 8,400 to 9,400 Daltons (which have a circulating half life of about 4-7 hours), the presence of larger, more hydrophobic, longer circulating half-life components of poloxamer 188 is not desirable. For example, among the desired activities of P188 is its rheologic effect to reduce blood viscosity and inhibit red blood cell (RBC) aggregation, which account for its ability to improve blood flow in damaged tissues. In contrast, more hydrophobic, higher molecular weight poloxamers such as P338 (also called Pluronic® F108) and P308 (Pluronic® F98), increase blood viscosity and RBC aggregation (Armstrong et al. (2001) Biorheology, 38:239-247). This is the opposite effect of P188 and indicates that higher molecular weight, hydrophobic poloxamer species can have undesirable biological effects. The results, thus, indicate that the hydrophobic components contained in the high molecular weight peak of purified (LCM-containing) poloxamer 188 are an unwanted impurity. Thus a poloxamer 188, such as LCMF poloxamer with a reduced amount of these components, is desirable.

### Example 2

### Pro-Hemostatic Effects of Poloxamer 188 (P188) in Fibrinolytic Plasma

a. Clotting Time

Poloxamer 188 (P188) LCMF described in Example 1 was assessed for its effects on hemostasis. Plasma samples from hospitalized patients treated with heparin (n=50) were collected from a clinical laboratory. LCMF poloxamer 188 (10 mg/mL), described in Example 1, was added to the plasma samples, and activated partial thromboplastin time (aPTT), HEPTEST® assay, and thrombin time assays were performed, using standard protocols.

Results are set forth in Table 1. The results show that the addition of P188 to plasma samples obtained from heparinized patients resulted in marked restoration of coagulation as indicated by the shortening of prolonged clotting times in the various assays.

TABLE 1

| Conditon | aPTT (sec) | HEPTEST ® (sec) | Thrombin time (sec) |
|---|---|---|---|
| Untreated | 95 ± 11 | 178 ± 23 | 223 ± 67 |
| P188-treated | 66 ± 8 | 90 ± 12 | 23 ± 3 |

b. Fibrin Assembly

The same plasma samples as described above also were analyzed for fibrin assembly using an optical density fibrinokinetic assay. See, e.g., Pratt et al., Biotechniques, 1992, 13(3): 430-433 and Tilley et al., Thrombosis Journal, 2011, 9:11, for detailed descriptions of fibrin assembly assays. Briefly, plasma samples from patients recieveing heparin therapy were supplemented with the LCMF poloxamer 188 (10.0 mg/mL) described in Example 1, dextran-70 (10.0 mg/mL), dextran-40 (10.0 mg/mL) or vehicle (saline). Untreated and treated plasma samples were loaded into wells of a microplate. Tissue thromboplastin (rabbit brain cephaloplastin), was added to each well, followed by calcium chloride to initiate the clotting reaction. The microplate was loaded into a standard spectrophotometer plate reader and the change in optical denisty at a wavelength of 405 nm was recorded every 5 seconds over 180 minutes.

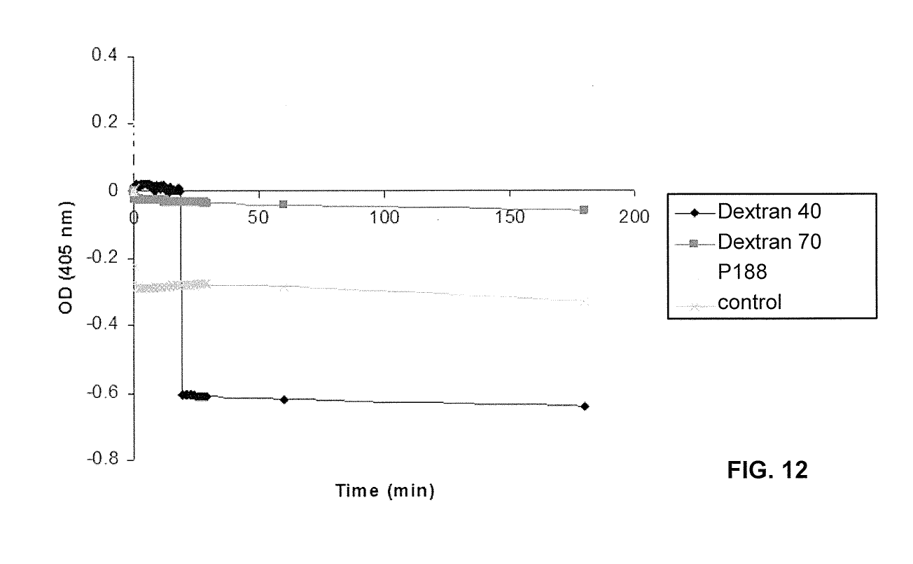

Results are set forth in FIG. 12. The results show that P188 supplementation of heparinized plasma restored fibrin assembly, as evidenced by a dramatic increase in optical density at 405 nm. In contrast, addition of saline or dextran polymers (dextran 40 and dextran 70) showed no similar ability to restore fibrin assembly.

c. Fibrin Assembly in Icteric Plasma

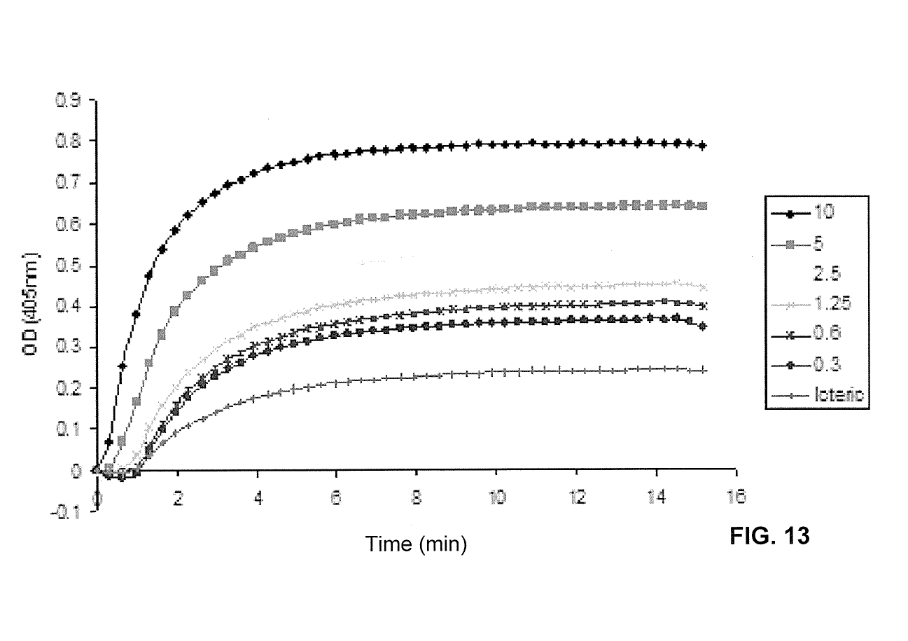

Patients with liver disease have impaired hemostatic function. To demosnstrate that P188 is useful to treat such dysfunction, unidentified, pooled icteric plasma samples were obtained from a hospital laboratory and tested using the fibrinokinetic assay described above. LCMF poloxamer 188, described in Example 1, was added to the icteric plasma at 0.3 mg/mL, 0.6 mg/mL, 1.25 mg/mL, 2.5 mg/mL, 5 mg/mL or 10 mg/mL. The results, which are depicted in FIG. 13, show that addition of P188 increased fibrin assembly in a concentration-dependent manner compared to the untreated icteric sample. Thus, poloxamer 188, can be used to treat hemostatic dysfunctions resulting from liver disease.

### Example 3

### Tissue Plasminogen Activator Bleeding Model

### Effect of Tranexamic Acid (TA) Treatment

Tissue plasminogen activator (t-PA) is used clinically to lyse occlusive thrombi. Bleeding complications that result from the unwanted lysis of hemostatic clots limits its use. TA is a synthetic derivative of the amino acid lysine that acts as a fibrinolytic inhibitor and is used clinically to antagonize unwanted bleeding. The effect oft-PA induced bleeding and its antagonism with TA was assessed in a rat tail bleeding model. Rats were anaesthetized by intraperitoneal injection of pentobarbital at 50 mg/kg. The thrombolytic agent t-PA was intravenously injected at a dose of 1 mg/kg, alone or in combination with TA at a dose of 5 mg/kg or 10 mg/kg. Another group of rats were administered 5 mg/kg TA alone. Control rats were administered saline. Each treatment group contained 7-10 rats.

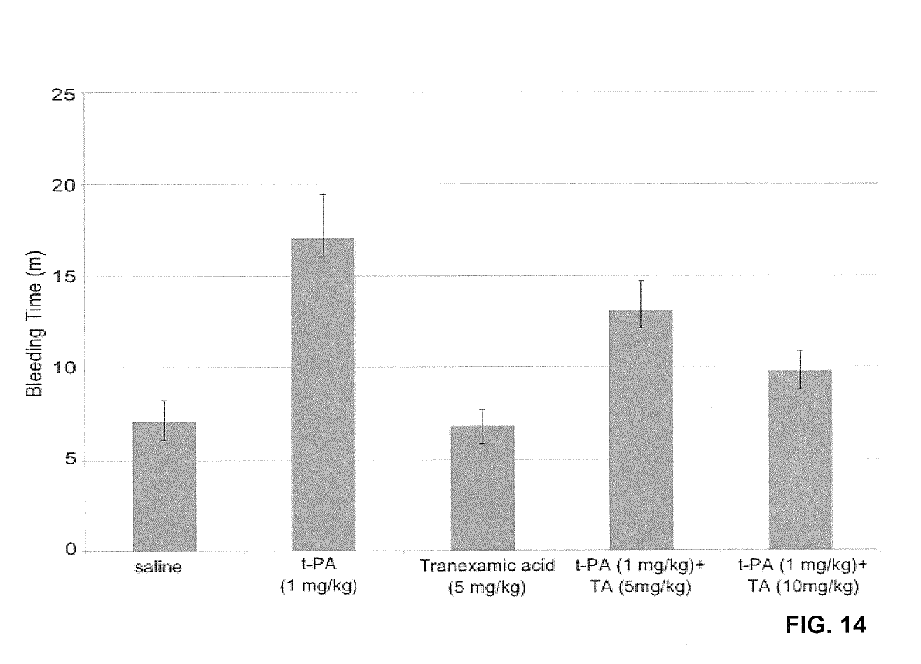

Following treatment, rat tails were amputated about 4 mm from the tip and the tails were immersed in normal saline at 37° C. The bleeding time was determined as the time for complete cessation of blood flow from the amputated tail by visual inspection. The results are set forth in FIG. 14. The results show that t-PA alone resulted in a marked prolongation of bleeding time. The combination oft-PA with TA showed a dose dependent reduction in the t-PA induced bleeding.

### Example 4

### Tissue Plasminogen Activator Bleeding Model

### Effect of Poloxamer 188 Treatment

The relative ability of P188 to antagonize t-PA induced bleeding (compared to TA) was studied by evaluating P188 in the same rat tail bleeding model as described in Example 3. Rats were anaesthetized by intraperitoneal injection of pentobarbital (50 mg/kg), and t-PA was intravenously injected at a dose of 1 mg/kg, alone or in combination with LCMF poloxamer 188, described in Example 1, at a dose of 1.25 mg/kg, 2.5 mg/kg or 10 mg/kg. Control rats were administered only saline. Each treatment group contained 7-10 rats.

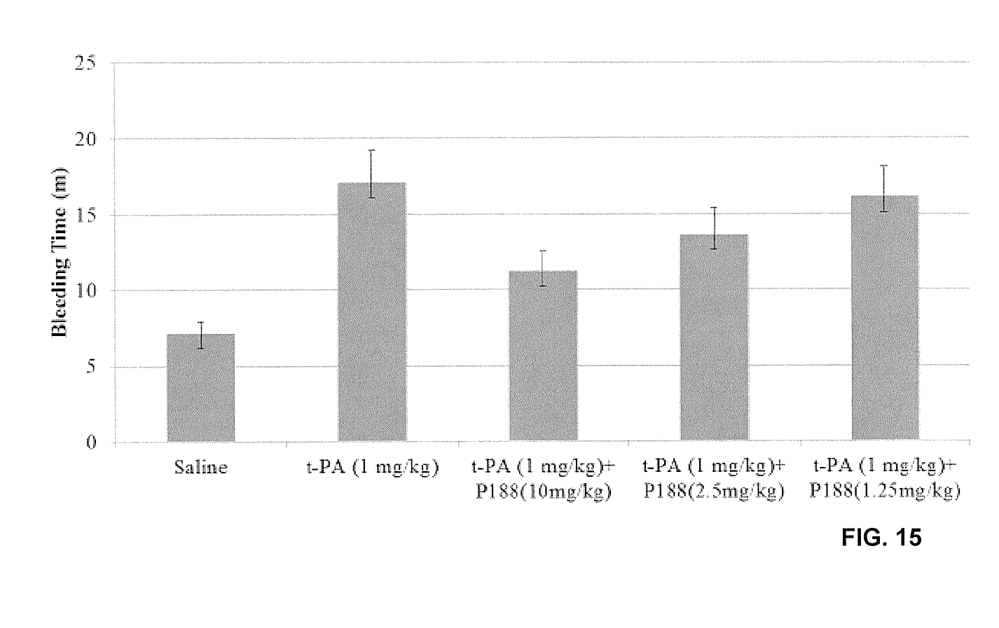

Rat tails were amputated about 4 mm from the tip and the tails were immersed in normal saline at 37° C. and bleeding time was assessed as described in Example 3. The results are depicted in FIG. 15. Consistent with the results in Example 3, the results show that treatment with t-PA alone increased bleeding time compared to saline injection. The addition of P188 to t-PA treated rats antagonized the t-PA-induced increase in bleeding time in a dose-dependent manner. The antagonization of the t-PA induced increase in bleeding time with P188 (10 mg/kg) was of a similar magnitude as that observed with TA (10 mg/kg) as described in Example 3 (compare FIG. 14 and FIG. 15).

### Example 5

### Poloxamer 188 (P188) in Combination with Tissue Plasminogen Activator (tPA) in Embolic Stroke Model

tPA is an effective treatment for acute stroke. Its use is limited by a narrow treatment window (3.5-4.5 hours from onset of stroke) and a greater risk of intracerebral hemnorrhage. This example shows that treatment, particularly pre-treatment with polaxamer (P188) in combination with tPA reduces these limitations by extending the window during which tPA is effective. To show this, the effects of administration of tPA alone, and tPA in combination with P188 were studied in a rat model of embolic middle cerebral artery occlusion. Male Wistar rats (N=10), 3-4 months old and weighing 350-400 g were subjected to embolic middle cerebral artery occlusion (MCAO) as described by Zhang (Zhang R L et al. A rat model of focal embolic cerebral ischemia. Brain Res. 1997, 766:83-92). Following occlusion, neurologic deficit was assessed 30 minutes after MCAO using a 5 point neurological scale (Longa scale; Longa et al., (1989) Stroke 20:84-91). The two groups (n=10/group) exhibited similar neurological deficits, with a mean score of slightly less than 2 by the Longa scale.

To compare the two treatments, P188 in combination with tPA and tPA alone were administered according to the schedule provided in Table 2. The dose of P188 was administered in a volume of 1.0 ml infused over a period of 1 hr by intravenous (IV) infusion via a tail vein. Both groups of rats received 10 mg/kg tPA (IV) at 4 hours following MCAO, with 10% of the dose administered as a bolus dose and the rest infused over 30 minutes via another tail vein. At 1 and 7 days after stroke onset (MCAO), clinical neurological function was assessed using the adhesive removal test and modified neurological severity score (mNSS) (Li Y, Chen J, Wang L, Lu M, Chopp M. Treatment of stroke with intracarotid administration of marrow stromal cells. Neurology 2001, 56: 1666-1672). Following clinical neurological assessment on day 7, animals were sacrificed and the brains were harvested for histological assessment of lesion volume and gross hemorrhage.

TABLE 2
Treatment schedule

| | Time Post-MCAO | | |
|---|---|---|---|
| | 3.5 hr | 4 hr | 9.5 hr |
| P188 + tPA | P188 | tPA | P188 |
| | (300 mg/kg, IV) | (10 mg/kg, IV) | (2,000 mg/kg |
| | | | (intraperitoneal) |
| tPA only | | tPA | |
| | | (10 mg/kg, IV)) | |

a. Clinical Neurological Assessment

i. Adhesive Removal Test

At days 1 and 7 after MCAO, both treatment groups were subjected to the adhesive removal test to investigate stroke related impairment of sensory and motor function. The test is based on the time required to remove an adhesive tape which rats will naturally remove from their body by grooming. The test tape was applied to the dorsal side of the paw, and the time of contact to time of removal was measured. At day 1, the rats treated with tPA alone removed the tape in an average time of approximately 120 seconds. Rats administered P188 and tPA demonstrated a significant reduction in time to tape removal (approximately 90 seconds on average; p<0.05). At day 7, the average time to tape removal for rats administered tPA alone was reduced to approximately 100 seconds, and the average time to tape removal for rats administered the P188 and tPA combination was again significantly reduced compared to the tPA treatment alone (approximately 65 seconds on average; p<0.05). indicating that the combination of P188 with t-PA was more effective in preserving sensory and motor function compared to tPA alone.

ii. Modified Neurological Severity Score (mNSS)

At 1 and 7 days after MCAO, rats in both treatment groups also were assessed by modified neurological severity score (mNSS), which uses a series of behavioral tests, including motor, sensory, reflex and balance tests (see, Chen et al., (2001) Stroke. 32(11):2682-2688). In the severity scores of injury, 1 score point is awarded for the inability to perform each test or for the lack of a tested reflex; thus, the higher the score, the more severe is the injury. At 1 day after MCAO, the group of rats treated with the combination of P188 and tPA had a significantly reduced average score compared to the group administered tPA alone (approximately 8.5 vs. approximately 10.5, respectively; p<0.05). At 7 days after MCAO, rats administered P188 and tPA also had a significantly reduced average score compared to rats administered only tPA (approximately 6 vs. approximately 8.5; p<0.05), indicating that the combination of P188 with tPA results in greater preservation of motor, sensory, reflex and balance compared to tPA alone.

iii. Lesion Volume

Following sacrifice, the brains of the rats were harvested and the lesion volumes as a result of the MCAO were measured on hematoxylin and eosin (H&E) stained coronal brain sections using a standard procedure (see, Swanson et al. (1990) A semi-automated method for measuring brain infarct volume. J Cereb Blood Flow Metab. 10:290-293.) Treatment with P188 in combination with tPA resulted in a significantly reduced average lesion volume compared to tPA treatment alone (approximately 20% of contralateral hemisphere vs. approximately 35% of contralateral hemisphere; p<0.05).

iv. Gross Hemorrhage

The harvested brains also were assessed for the incidence of gross hemorrhage, defined as blood evident to the unaided eye on the H&E stained coronal sections. Of the animals treated with P188 and tPA, 20% (2 out of 10) of the animals exhibited gross hemorrhage. Of the animals treated with tPA alone, 30% (3 out of 10) of the animals exhibited gross hemorrhage. Thus, the combination treatment of P188 with tPA reduced the incidence of gross hemorrhage compared to tPA alone.

### Example 6

### Poloxamer 188 Treatment of Heparin-Induced Hemostatic Dysfunction In Vivo

A 65 year old female patient with end stage renal disease undergoes placement of a standard wall polytetrafluorethylene graft (diameter 5.0-7.0 mm) in the upper arm between an artery and a vein for dialysis access. The patient is treated with unfractionated heparin administered intravenously at a dose of 3,000 IU prior to placement of the vascular clamps required for anastomoses. Anastomoses is performed using 6-0 polypropylene sutures on BV-1 needles. After anastomoses, the arterial and venous anastomoses sites are treated topically with 2.0 mL of standard 2-chambered fibrin glue containing fibrinogen (60 mg/mL) and thrombin (500 NIH U/mL with 40 nmol calcium chloride). The fibrin glue is allowed to polymerize for 60 seconds. During that time, and prior to removal of the vascular clamps, the patient also receives a single intravenous infusion of a P188, such as LCMF P188 described in Example 1, at a dose of 400 mg/kg. Higher doses can be administered. The activated partial thromboplastin time (aPTT) is measured prior to the procedure, following the dose of heparin, and approximately 5 minutes after the infusion of P188. Following removal of the vascular clamps, hemostasis at the points of anastomoses and perfusion through the graft are evaluated.

Prior to the procedure, the aPTT should be about 24.7 sec. Following the dose of heparin, the aPTT should increase to 63.1 sec. After the infusion of P188, the aPTT, based on the results in the above examples and the description herein, will be reduced, such as to 33 sec. As a result, the patency of the graft will be excellent.

### Example 7

### Poloxamer 188 Treatment of Bleeding Induced by t-PA and Heparin In Vivo

A 69 year old male patient with an occlusion of a superficial femoral artery is treated with tissue plasminogen activator (t-PA) administered intra-arterially through an end hole catheter as a continuous dose of 1.0 mg/kg/hr plus heparin as a continuous infusion to raise the activated partial thromboplastin time (aPTT) to 1.5× normal. After 8 hours of infusion, the resolution of the occlusion is assessed by angiogram and the bleeding at the site of the arterial puncture is examined. The patient has developed significant bleeding that is not controlled with direct pressure at the site of arterial puncture, and is administered poloxamer 188, such as LCMF P188 described in Example 1, at a dose of at least 400 mg/kg, as an intravenous infusion, over one hour or suitable time based on the concentration of the poloxamer in the infusion composition, following discontinuation of the heparin and t-PA infusion. This will control the bleeding. Following P188 infusion, and demonstration that the bleeding is controlled, with only a slightly prolonged aPTT, the previous dose oft-PA is resumed. A repeat angiogram is performed 4 hours later to assess the status of the occlusion. Perfusion and the patient's popliteal and pedal pulse also are monitored.

### Example 8

### In Vivo Topical Application of Poloxamer 188 and a Fibrin Glue

A 60 year old male patent with end stage renal disease undergoes placement of a standard-wall polytetrafluorethylene graft (diameter 5.0-7.0 mm) placed in the upper arm between an artery and a vein for dialysis access. Anastomoses is performed using 6-0 polypropylene sutures on BV-1 needles. The patient is treated with unfractionated heparin administered intravenously at a dose of 3,000 IU prior to placement of the vascular clamps required for performance of the anastomoses. Following anastomoses, approximately 1.0 mL of a standard 2-chambered fibrin glue, containing fibrinogen (60 mg/mL) and thrombin (500 NIH U/mL with 40 nmol calcium chloride), is applied topically to the venous suture site. The arterial suture site is treated in an identical manner with the same 2-chambered fibrin glue formulation, except the fibrinogen chamber is supplemented with poloxamer 188, such as LCMF P188 described in Example 1, at a concentration of 10.0 mg/mL. The fibrin glue is allowed to polymerize for 60 seconds, following which the vascular clamps are removed. The time to hemostasis is determined, and compared, for the venus and arterial anastomoses sites immediately after the time of clamp release and at every minute thereafter until complete hemostasis is achieved at the venus and arterial suture sites. Based on the results shown herein and the description herein, the anastomoses site treated with the poloxamer 188 supplemented fibrin glue will achieve complete hemostasis at the time of clamp release. Complete hemostasis at the site treated with standard fibrin glue will be achieved about 4 minutes after clamp release.

Since modifications will be apparent to those of skill in this art, it is intended that this invention be limited only by the scope of the appended claims.

1. A method for ameliorating hemostatic dysfunction or preventing it, comprising:
administering to a subject experiencing hemostatic dysfunction or to a subject who is undergoing treatment that can result in hemostatic dysfunction an amount of a polyoxyethylene/polyoxypropylene copolymer to restore, reduce or prevent the hemostatic dysfunction, thereby restoring or improving hemostasis, wherein:
the polyoxyethylene/polyoxypropylene copolymer has the formula:
HO(CH₂CH₂O)_{a′}—[CH(CH₃)CH₂O]_{b}—(CH₂CH₂O)ₐH wherein:
a′ and a are the same or different and each is an integer, whereby the hydrophile portion represented by (C₂H₄O) constitutes approximately 60% to 90% or 60%-90% by weight of the compound;
b is an integer, whereby the hydrophobe represented by (C₃H₆O) has a molecular weight of about 1,200 Da to about 2,300 Da or 1,200 to 2,300 Da;
the copolymer preparation has been purified to remove low molecular weight impurities; and
the amount of copolymer administered achieves a circulating Cₘₐₓ concentration of greater than about 1.0 mg/ml.

2. The method of claim 1, wherein:
the copolymer has the formula:
HO(C₂H₄O)_{a′}—(C₃H₆O)_{b}—(C₂H₄O)ₐH;
a′ and a are the same or different and each is an integer from 70 to 105, inclusive; and
b is an integer from 15 to 75, inclusive.

3. The method of claim 1, wherein the hydrophobe represented by (C₃H₆O) has a molecular weight of about 1,400 Da to 2,000 Da or 1,400 Da to 2,000 Da, and the hydrophile portion constitutes approximately 70% to 90% or 70% to 90% by weight of the copolymer.

4. The method of claim 1, wherein the molecular weight of the hydrophobe portion (C₃H₆O) is approximately or is 1,750 Da and the total molecular weight of the copolymer is approximately or is 8,400 to 8,800 Da.

5. The method of claim 1, wherein the copolymer comprises a poloxamer 188.

6. The method of claim 1, wherein the copolymer has reduced impurities, whereby the polydispersity value is less than or equal to 1.07.

7. The method of claim 1, wherein no more than 1.5% or 1% of the total components in the distribution of the copolymer are low molecular weight components having an average molecular weight of less than 4,500 Daltons.

8. The method of claim 1, wherein the polyoxyethylene/polyoxypropylene copolymer has the formula:
HO(C₂H₄O)_{a′}—(C₃H₆O)_{b}—(C₂H₄O)ₐH, wherein:
a′ and a are the same and are 78, 79 or 80; and
b is 27, 28, 29 or 30.

9. The method of claim 1, wherein the copolymer is a long-circulating material-free (LCMF) poloxamer.

10. The method of claim 9, wherein:
the LCMF poloxamer is a polyoxyethylene/polyoxypropylene copolymer that has the formula HO(CH₂CH₂O)_{a′}—[CH(CH₃)CH₂O]_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe (C₃H₆O) is between approximately 1,300 and 2,300 Daltons;
no more than 1.5% of the total components in the polymeric distribution of the copolymer are low molecular weight components having an average molecular weight of less than 4,500 Daltons;
no more than 1.5% of the total components in the polymeric distribution of the copolymer are high molecular weight components having an average molecular weight of greater than 13,000 Daltons;
the polydispersity value of the copolymer is less than approximately 1.07 or less than 1.07; and
following intravenous administration to a human subject, the circulating plasma half-life of any components not comprising the main peak in the distribution of copolymer is no more than 5.0-fold the circulating half-life of the main component in the distribution of the copolymer.

11. The method of claim 10, wherein all components comprising the polymeric distribution of the poloxamer copolymer have a circulating half-life in the plasma of the subject that is no more than 4.0-fold, or 3.0-fold longer than the circulating half-life of the main component of the copolymer following intravenous administration to a subject.

12. The method of claim 10, wherein all components in the distribution of the copolymer, when administered to a human subject, have a half-life in the plasma of the subject that is no more than 10 or 12 hours.

13. The method of claim 10, wherein the average molecular weight of the polyoxyethylene/polyoxypropylene copolymer is 8,400-8,800 Daltons.

14. The method of claim 10, wherein:
the LCMF poloxamer is a polyoxyethylene/polyoxypropylene copolymer that has the formula HO(CH₂CH₂O)_{a′}—[CH(CH₃)CH₂O]_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe (C₃H₆O) is between approximately 1,300 and 2,300 Daltons;
no more than 1.5% of the total components in the distribution of the copolymer are low molecular weight components having an average molecular weight of less than 4,500 Daltons;
no more than 1.5% of the total components in the distribution of the copolymer are high molecular weight components having an average molecular weight of greater than 13,000 Daltons;
the polydispersity value of the copolymer is less than approximately 1.07 or less than 1.07; and
the LCMF poloxamer is more hydrophilic than a purified poloxamer 188 that contains the long circulating material (LCM).

15. The method of claim 9, wherein:
the LCMF poloxamer is a polyoxyethylene/polyoxypropylene copolymer that has the formula HO(CH₂CH₂O)_{a′}—(CH(CH₃)CH₂O)_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe (C₃H₆O) is between approximately 1,300 and 2,300 Daltons;
no more than 1.5% of the total components in the distribution of the copolymer are low molecular weight components having an average molecular weight of less than 4,500 Daltons;
no more than 1.5% of the total components in the distribution of the copolymer are high molecular weight components having an average molecular weight of greater than 13,000 Daltons;
the polydispersity value of the copolymer is less than approximately 1.07 or less than 1.07;
the LCMF poloxamer has a mean retention time (t_{R}) as assessed by reverse phase-high performance liquid chromatography (RP-HPLC) that is shorter than purified LCM-containing poloxamer 188 under the same RP-HPLC conditions; and
the capacity factor (k′) of the LCMF poloxamer as assessed by RP-HPLC is less than the k′ for purified LCM-containing poloxamer 188 under the same RP-HPLC conditions.

16. The method of claim 9, wherein the copolymer is produced by a method comprising
a) introducing a poloxamer 188 solution into an extractor vessel, wherein the poloxamer is dissolved in a first alkanol to form a solution;
b) admixing the poloxamer solution with an extraction solvent comprising a second alkanol and supercritical carbon dioxide under a temperature and pressure to maintain the supercritical carbon dioxide for a first defined period, wherein:
the temperature is above the critical temperature of carbon dioxide but is no more than 40° C.;
the pressure is 220 bars to 280 bars; and
the alkanol is provided at an alkanol concentration that is 7% to 8% by weight of the total extraction solvent;
c) increasing the concentration of the second alkanol in step b) in the extraction solvent a plurality of times in gradient steps over time of the extraction method, wherein:
each plurality of times occurs for a further defined period; and
in each successive step, the alkanol concentration is increased 1-2% compared to the previous concentration of the second alkanol; and
d) removing the extraction solvent from the extractor vessel to thereby remove the extracted material from the raffinate poloxamer preparation.

17. The method of claim 16, wherein:
in step a), the ratio of poloxamer to first alkanol, by weight, is about or is from 2:1 to 3:1, inclusive; and
the plurality of times in step c) occurs in two, three, four or five gradient steps.

18. The method of claim 1, wherein hemostatic dysfunction is manifested as increased bleeding, prolonged blood clotting times or is a parameter associated with a risk of increased bleeding.

19. The method of claim 1, wherein the hemostatic dysfunction is manifested as increased bleeding or risk thereof, wherein the bleeding is internal or external.

20. The method of claim 1, wherein the hemostatic dysfunction results from surgery or trauma or a clotting disorder.

21. The method of claim 1, wherein the amount of the copolymer administered achieves a Cₘₐₓ concentration of at least 10 mg/ml.

22. The method of claim 1, wherein the dosage of the copolymer is at least 400 mg/kg or 400-1000 mg/kg or 800-1200 mg/kg or 800-1000 mg/kg.

23. The method of claim 1, wherein the copolymer composition is administered intravenously or subcutaneously or by inhalation.

24. The method of claim 23, wherein the copolymer composition is administered intravenously over the course of 1-6 hours.

25. The method of claim 1, wherein the subject has undergone or is undergoing pharmacological thrombolytic therapy.

26. The method of claim 25, wherein the subject has experienced an episode for which thrombolytic therapy is administered, wherein the episode is selected from among a myocardial infarction, a thromboembolic stroke, pulmonary embolism, a deep vein thrombosis, an arterial thrombus and a venous thrombus.

27. The method of claim 25, wherein the pharmacological thrombolytic therapy is effected by treatment with one or more of a tissue plasminogen activator (t-PA), anistreplase, streptokinase, urokinase, or a direct acting thrombolytic.

28. The method of claim 26, wherein pharmacological thrombolytic therapy is tissue plasminogen activator (t-PA).

29. The method of claim 26, wherein:
the pharmacological thrombolytic therapy is direct acting pharmacological thrombolytic therapy; and
the direct acting pharmacological thrombolytic therapy comprises administration of plasmin.

30. The method of claim 25, wherein the polyoxyethylene/polyoxypropylene copolymer is administered before or concomitantly with the pharmacological thrombolytic therapy.

31. The method of claim 25, wherein the polyoxyethylene/polyoxypropylene copolymer is administered after the pharmacological thrombolytic therapy.

32. The method of claim 25, wherein:
the polyoxyethylene/polyoxypropylene copolymer is administered in two doses; and
the first dose is administered concomitantly with the pharmacological thrombolytic therapy.

33. The method of claim 30, wherein the first dose of the polyoxyethylene/polyoxypropylene copolymer is administered prior to the pharmacological thrombolytic therapy.

34. The method of claim 33, wherein a second dose of the polyoxyethylene/polyoxypropylene copolymer is administered after the pharmacological thrombolytic therapy.

35. The method of claim 34, wherein the second dose of the polyoxyethylene/polyoxypropylene copolymer is administered between 30 minutes and 10 hours after the pharmacological thrombolytic therapy.

36. The method of claim 25, wherein the subject is or has been treated with anti-coagulants.

37. The method of claim 25, wherein the treatment results in a concentration of the polyoxyethylene/polyoxypropylene copolymer in the circulation of the subject of at least 10 mg/mL.

38. The method of claim 1, wherein the subject has had an acute ischemic stroke (AIS) or a hemorrhagic stroke.

39. The method of claim 25, wherein:
the subject has had an acute ischemic stroke (AIS); and
the pharmacological thrombolytic therapy is administered immediately after the AIS up to about 10 hours after the AIS.

40. The method of claim 39, wherein the pharmacological thrombolytic therapy is administered 3.5 hours after the AIS and up to about 10 hours after the AIS.

41. The method of claim 25, wherein a dose of polyoxyethylene/polyoxypropylene copolymer is administered before the pharmacological thrombolytic therapy.

42. A method for extending the pharmacologic therapy treatment window for ischemic stroke, comprising:
administering a polyoxyethylene/polyoxypropylene copolymer to a subject after the stroke to extend the treatment for ischemic stroke; and
then administering the pharmacological thrombolytic therapy.

43. The method of claim 42, further comprising repeating administration of the polyoxyethylene/polyoxypropylene copolymer after administration of the pharmacological thrombolytic therapy.

44. The method of claim 42, wherein the pharmacological thrombolytic therapy is effected by treatment with one or more of a tissue plasminogen activator (t-PA), anistreplase, streptokinase, urokinase, or a direct acting thrombolytic.

45. The method of claim 44, wherein pharmacological thrombolytic therapy is tissue plasminogen activator (t-PA).

46. The method of claim 44, wherein the pharmacological thrombolytic therapy is direct acting pharmacological thrombolytic therapy that comprises administration of plasmin.

47. The method of claim 43, wherein the first polyoxyethylene/polyoxypropylene copolymer treatment is administered within 3.5 hours after the stroke.

48. The method of claim 47, wherein thrombolytic therapy is administered thereafter, but before 5, 4.5, or 4 hours after the stroke.

49. The method of claim 48, comprising a further treatment with the poloxamer copolymer at least 6, 7, 8, 9 or 10 hours after the stroke and after the thrombolytic therapy.

50. The method of claim 42, wherein the subject has had an acute ischemic stroke.

51. The method of claim 50, wherein the pharmacological thrombolytic therapy is administered immediately after the AIS up to about or 10 hours after the AIS.

52. The method of claim 50, wherein the pharmacological thrombolytic therapy is administered at least 3.5 hours after the AIS up to about or 10 hours after the AIS; or between 3 and 5 hours after the AIS, or about 6 hours to about 10 hours after the AIS.

53. The method of claim 42, wherein the polyoxyethylene/polyoxypropylene copolymer is administered at a dosage to result in the circulation in the subject of from about or at 0.05 mg/mL to about or at 10 mg/mL or of from about 0.2 mg/mL to about 4.0 mg/mL or least 0.5 mg/mL.

54. The method of claim 42, wherein:
the polyoxyethylene/polyoxypropylene copolymer has the chemical formula
HO(C₂H₄O)_{a′}—(C₃H₆O)_{b}—(C₂H₄O)ₐH;
a′ and a are the same or different and each is an integer, whereby the hydrophile portion represented by (C₂H₄O) constitutes approximately 60% to 90% or 60%-90% by weight of the compound; and
b is an integer, whereby the hydrophobe represented by (C₃H₆O) has a molecular weight of about 1,200 Da to about 2,300 Da or 1,200 to 2,300 Da.

54. (canceled)

55. The method of claim 42 wherein the polyoxyethylene/polyoxypropylene copolymer is a long-circulating material-free (LCMF) poloxamer.

56. The method of claim 55, wherein:
the LCMF poloxamer is a polyoxyethylene/polyoxypropylene copolymer that has the formula HO(CH₂CH₂O)_{a′}—[CH(CH₃)CH₂O]_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe (C₃H₆O) is between approximately 1,300 Daltons and 2,300 Daltons;
no more than 1.5% of the total components in the polymeric distribution of the copolymer are low molecular weight components having an average molecular weight of less than 4,500 Daltons;
no more than 1.5% of the total components in the polymeric distribution of the copolymer are high molecular weight components having an average molecular weight of greater than 13,000 Daltons;
the polydispersity value of the copolymer is less than approximately 1.07 or less than 1.07; and
following intravenous administration to a human subject, the circulating plasma half-life of any components not comprising the main peak in the distribution of copolymer is no more than 5.0-fold the circulating half-life of the main component in the distribution of the copolymer.

57. The method of claim 10, wherein:
the LCMF poloxamer is a polyoxyethylene/polyoxypropylene copolymer that has the formula HO(CH₂CH₂O)_{a′}—[CH(CH₃)CH₂O]_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe (C₃H₆O) is between approximately 1,300 and 2,300 Daltons;
no more than 1.5% of the total components in the distribution of the copolymer are low molecular weight components having an average molecular weight of less than 4,500 Daltons;
no more than 1.5% of the total components in the distribution of the copolymer are high molecular weight components having an average molecular weight of greater than 13,000 Daltons;
the polydispersity value of the copolymer is less than approximately 1.07 or less than 1.07; and
the LCMF poloxamer 188 is more hydrophilic than a purified poloxamer 188 that contains the long circulating material (LCM).

58. The method of claim 42, wherein the amount of the polyoxyethylene/polyoxypropylene copolymer administered achieves a Cₘₐₓ concentration of greater than 0.5 mg/ml or at least 1 mg/ml.

59. The method of claim 42, wherein the dosage of the polyoxyethylene/polyoxypropylene copolymer is at least about 20 mg/kg, or at least about 50 mg/kg or is at least 100 mg/kg or at least about 100 mg/kg or is at least 150 mg/kg or at least about 150 mg/kg or between about 50-1200 mg/kg.

60. The method of claim 42, wherein the polyoxyethylene/polyoxypropylene copolymer comprises a poloxamer 188.
